(19) United States
(12) Patent Application Publication
(10) Pub. No.: US 2015/0290437 A1
(43) Pub. Date: Oct. 15, 2015

(54) LUMINAL DEVICE DELIVERY SYSTEM

(71) Applicant: ArtVentive Medical Group, Inc., San Marcos, CA (US)

(72) Inventors: Leon RUDAKOV, San Marcos, CA (US); Andrew BLACK, Johnsburg, IL (US); Andrew R. LEOPOLD, Hawthorn Woods, IL (US); Brandon HUGGINS, North Barrington, IL (US); Kelly JENSEN, Palatine, IL (US)

(21) Appl. No.: 14/749,565

(22) Filed: Jun. 24, 2015

Related U.S. Application Data
(63) Continuation of application No. PCT/US2015/015945, filed on Feb. 13, 2015.
Application No. PCT/US2015/015945 is a continuation-in-part of application No. 14/304,868, filed on Jun. 13, 2014.
(60) Provisional application No. 62/088,517, filed on Dec. 5, 2014.
(60) Provisional application No. 61/939,659, filed on Feb. 13, 2014.

(51) Int. Cl. A61M 25/10; A61F 2/958

(52) U.S. Cl. CPC A61M 25/10; A61F 2/958; A61F 2002/9505

(57) ABSTRACT

An implant may include a frame and a cover to facilitate endoluminal vessel occlusion, selective release of embolic material toward a target region, and/or endoluminal stenting. The frame of the implant provides radial expansion properties to secure the cover within a body vessel. The cover and/or the frame can reduce or occlude flow of a fluid through the body vessel.

### CROSS-REFERENCE TO RELATED APPLICATIONS

This application is a continuation of International Application No. PCT/US2015/015945, filed on Feb. 13, 2015, which is an international application designating the United States and claims the benefit of and priority to U.S. Provisional Application No. 62/088,517, filed on Dec. 5, 2014, U.S. Provisional Application No. 61/939,659, filed on Feb. 13, 2014, and U.S. patent application Ser. No. 14/304,868, filed on Jun. 13, 2014, the entirety of the disclosures of each of which is incorporated herein by reference.

### FIELD

The subject technology relates generally to apparatuses and methods for blood vessel occlusion and vascular stenting.

### BACKGROUND

Rapid, well-controlled, and safe methods to limit bleeding in vessels have encouraged the development of endovascular devices and techniques, and their introduction into clinical practice. Early devices used balloons, either non-detachable or later detachable, in order to block vessels, for example, in the treatment of carotid-cavernous fistulas and saccular aneurysms.

Typically made from latex or silicone, balloons are delivered to a desired location in a vessel, then inflated in order to physically occlude the vessel. While other devices have since been developed, balloon occlusion remains in use, and is indicated for use in treating a variety of life-threatening conditions, including for example, giant cerebral and skull base aneurysms, traumatic and non-traumatic vessel injury or rupture, vertebro-vertebral arteriovenous fistulas, and pre-operative tumor resections.

Detachable balloons are also useful clinically in procedures outside of neurological intervention. For example, balloons can be useful in flow reduction procedures such as shunt occlusion in patients with transjugular intrahepatic portosystemic shunts and hepatic insufficiency, intrahepatic arterioportal fistulas, treatment of varicoceles, shunt occlusion in patients with a Blalock-Taussig shunt, obliteration of pulmonary arteriovenous fistulas, arteriovenous malformations or aortopulmonary anastomoses, coronary arteriovenous fistulas, or renal arteriovenous fistulas. Detachable balloons are also used in preoperative devascularization before surgical resection of organs such as the kidney.

SUMMARY

Some embodiments provided herein relate to vessel occlusion by delivery of radially expandable implant frames that achieve immediate total occlusion of blood flow. Frame configurations, expected delivered and expanded dimensions, and a description of target anatomy of some embodiments is provided.

Additionally, some embodiments provided herein relate to implantation in small blood vessels, such as from about 3 mm to about 20 mm, from about 5 mm to about 15 mm, or from about 7 mm to about 11 mm. The target delivery profile can be from about 2 Fr to about 6 Fr, and in some embodiments, from about 3 Fr to about 5 Fr.

Further embodiments can provide vascular stenting for vessels that are from about 3 mm to about 16 mm, from about 5 mm to about 13 mm, and in some embodiments, from about 7 mm to about 11 mm. The target delivery profile can be from about 2 Fr to about 8 Fr, about 3 Fr to about 7 Fr, from about 4 Fr to about 6 Fr, or in some embodiments, about 5 Fr. Additionally, expansion of the implant can provide sufficient radial force against the inside wall of a vein. Some embodiments can comprise features or means configured to minimize backflow of blood or minimize venous insufficiency. For example, treatment applications for embodiments of the device can include ilio-femoral venous obstruction and chronic iliac venous outflow obstruction as a result of venous disease.

Embodiments of the implants provided herein can be manufactured via several methods including shape-setting of drawn wire, chemical etching of a NiTi sheet of material, laser cutting of a tubular member, such as a material sheet or tubing, and/or electrical discharge machining (EDM) of a tubular member, such as a material sheet or tubing.

The implants disclosed herein can comprise flexible and/or shape memory materials such that they may be distorted from an expanded shape to a smaller diameter or straight shape to allow for delivery to a target location by way of a minimally invasive catheter-based approach.

In accordance with some embodiments, the implant can comprise a frame and a cover material. The cover material can comprise ePTFE tubing, film, and/or suture for attachment purposes. Additionally, the cover material may be fibrous, mesh-like, or impermeable in density.

The implant frame and/or implant cover can comprise a collagen coating or collagen treatment to improve anchoring of the implant in the target vessel. The collagen can be configured to promote cell adhesion to implant materials, thereby facilitating improved support for the implant and vessel structure while acting as an anti-migration feature for the implant.

The implant frame can comprise a straight or constant diameter, a tapering diameter, or sections of variable diameter extending over its length, which can facilitate anchoring within a vessel and optimal deployment function.

Embodiments of the systems and devices disclosed herein address the unmet need for a device that can provide a fast, precise and reliable way to close a bodily lumen. The endoluminal occlusion system can include two major subsystems: a guide sheath assembly and an implant carrier assembly. The implant carrier assembly can include an implant device and a handle assembly. Embodiments of the present disclosure can also comprise various features disclosed in U.S. Pat. No. 8,328,840, issued on Dec. 11, 2012, the entirety of the disclosure of which is incorporated herein by reference.

A single wire can be shaped in a back-and-forth pattern around a circumference. The shape can be set to an expanded diameter to fill the circumference of a blood vessel. The ends of the wire can be welded or otherwise attached such that there is a continuous construct around the full circumference. The design can be intended to allow a high ratio of expansion, while maintaining a radial force at all points around the circumference of the blood vessel in order to seal blood flow. The construct can be covered with a non-permeable material, sealed at one or both ends to occlude blood flow. The cover can be silicone rubber, ePTFE, or urethane, and designed to have a tight fit around the expanded construct. The construct size can be chosen based on endoluminal size at the implant location, expected to be a minimum 25% greater in diameter than the endoluminal diameter.

An expandable feature (braid, balloon, or other construct) with a non-permeable cover can be attached to a filament shaped into a coil. The expandable feature can utilize shaped or otherwise positioned wires such that axial compression of the expandable feature causes a diameter increase intended for occlusion of a blood vessel. The expandable feature can alternatively increase in diameter by internal pressure caused by an expandable gel or other material, or insertion of liquid. The coil can be shape set to a corresponding diameter relative to the expandable feature, and acts to anchor the expandable occlusion feature within a blood vessel or vascular malformation such as fistula, etc.

According to some embodiments, medical methods and apparatuses are provided for controlling or modifying a pressure gradient between blood vessels. Further, some embodiments can provide an adjustable implant that can be modified to provide a desired pressure gradient. The implant can be adjusted from a first non-zero flow rate to a second non-zero flow rate, and in some embodiments, from the second non-zero flow rate to a third non-zero flow rate, to provide a variety of gradient options. For example, the implant can be modified in situ. Further, some embodiments can provide methods and implants for adjusting a hepatic venous pressure gradient (HVPG) between the portal and hepatic veins in a transjugular intrahepatic portosystemic procedure. Such methods and apparatuses can be configured to adjust or maintain the HVPG equal to or below about 10 mmHg.

Frame configurations, expected delivered and expanded dimensions, and a description of target anatomy of some embodiments are provided. Aspects of implants, catheters, and delivery devices that can be utilized in combination with the implants, systems, methods, and features disclosed herein are disclosed in: U.S. patent application Ser. No. 12/826,593, filed on Jun. 29, 2010 (086538-0012); U.S. patent application Ser. No. 13/367,338, filed on Feb. 6, 2012 (086538-0018); U.S. patent application Ser. No. 12/906,993, filed on Oct. 18, 2010 (086538-0014); U.S. patent application Ser. No. 13/828,974, filed on Mar. 14, 2013 (086538-0030); U.S. Patent Application No. 61/836,061, filed on Jun. 17, 2013 (086538-0038); U.S. patent application Ser. No. 14/044,794, filed on Oct. 2, 2013 (086538-0039); U.S. patent application Ser. No. 14/281,797, filed on May 19, 2014 (086538-0055); U.S. Patent App. No. 61/835,406, filed on Jun. 14, 2013 (086538-0032); U.S. Patent App. No. 61/904,376, filed on Nov. 14, 2013 (086538-0041); U.S. Patent App. No. 61/904,379, filed on Nov. 14, 2013 (086538-0043); U.S. Patent App. No. 61/835,461, filed on Jun. 14, 2013 (086538-0034); U.S. Patent App. No. 61/900,321, filed on Nov. 5, 2013 (086538-0040); U.S. patent application Ser. No. 14/101,171, filed on Dec. 9, 2013 (086538-0046); U.S. Patent App. No. 61/987,446, filed on May 1, 2014 (086538-0054); and U.S. patent application Ser. No. 14/304,868, filed on Jun. 13, 2014 (086538-0057), the entireties of which are incorporated herein by reference.

Some embodiments can provide vascular implantation for vessels that are from about 2 mm to about 16 mm, from about 5 mm to about 13 mm, and in some embodiments, from about 7 mm to about 11 mm. The target delivery profile can be from about 2 Fr to about 8 Fr, about 3 Fr to about 7 Fr, from about 4 Fr to about 6 Fr, or in some embodiments, about 5 Fr. Additionally, expansion of the implant can provide sufficient radial force against the inside wall of a vein. Some embodiments can comprise features or means configured to minimize backflow of blood or minimize venous insufficiency. For example, treatment applications for embodiments of the implant can include ilio-femoral venous obstruction and chronic iliac venous outflow obstruction as a result of venous disease.

The implant may serve as a calibrated flow and pressure reduction tool in some embodiments. Some embodiments of the implant can be used for purposes of tumor devascularization, reducing traumatic bleeding or hemorrhage, high-flow vascular malformations, vascular or airway volume reduction procedures, treatment of a target lesion, treatment and embolization of incompetent venous systems in low extremities (i.e., legs and lower abdominal area), treatment varicose veins in the leg (i.e., great saphenous vein and spider veins in deeper system), attending to other indications such as arterio-venous malformation (AVM), pelvic varices, etc.

Further, some embodiments provide an implant delivery system that comprises a catheter having a flexible, torque-resistant tip over which an implant frame may be secured and delivered to a target treatment site. Some embodiments also relate to engagement mechanisms whereby an implant can be engaged relative to a delivery catheter and actuation mechanisms for releasing the implant from the engagement.

Further, some embodiments provided herein can be used to provide temporary or permanent occlusion of a vessel during and/or after treatment of a tumor by intravascular injection of fluids, chemotherapy drugs, liquid embolic agents, and/or other therapeutic agents delivered into the feeding vessels and/or into the tumor.

Some embodiments of the implants provided herein can be manufactured via several methods including shape-setting of drawn wire, chemical etching of a NiTi sheet of material, laser cutting of a tubular member, such as a material sheet or tubing, and/or electrical discharge machining (EDM) of a tubular member, such as a material sheet or tubing.

The implants disclosed herein can comprise flexible and/or shape memory materials such that they may be distorted from an expanded shape to a smaller diameter or straight shape to allow for delivery to a target location by way of a minimally invasive catheter-based approach.

In accordance with some embodiments, the implant can comprise a frame and a cover material. The cover material can comprise ePTFE tubing, film, and/or suture for attachment purposes. Additionally, the cover material may be fibrous, mesh-like, or impermeable in density.

The implant frame and/or implant cover can comprise a collagen coating or collagen treatment to improve anchoring of the implant in the target vessel. The collagen can be configured to promote cell adhesion to implant materials, thereby facilitating improved support for the implant and vessel structure while acting as an anti-migration feature for the implant.

The implant frame can comprise a straight or constant diameter, a tapering diameter, or sections of variable diameter extending over its length, which can facilitate anchoring within a vessel and optimal deployment function.

Further, in some embodiments, the implant can comprise a one-way valve that can allow fluids to pass for injection, but minimizing backflow of those fluids into the systemic vasculature.

Embodiments of the systems and devices disclosed herein address the unmet need for a device that can provide a fast, precise and reliable way to close a bodily lumen. The endoluminal occlusion system can include two major subsystems: a guide sheath assembly and an implant carrier assembly. The implant carrier assembly can include an implant device and a handle assembly. Embodiments of the present disclosure can also comprise various features disclosed in U.S. Pat. No. 8,328,840, issued on Dec. 11, 2012, the entirety of the disclosure of which is incorporated herein by reference.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 55. The other clauses can be presented in a similar manner.

Clause 1. A medical device delivery system, comprising: a catheter comprising proximal and distal portions, first and second engagement sections at the distal portion, and an actuator lumen extending from the proximal portion to a first aperture at the first engagement section of the distal portion; an elongate actuation member, longitudinally moveable within the actuator lumen, comprising a distal coupling section; and a medical device extending along an outer surface of the catheter distal portion in a collapsed configuration, the device having (i) a proximal engagement portion, releasably coupled to the second engagement section, and (ii) a distal engagement portion extending proximally through the first aperture into the actuator lumen toward the distal coupling section to longitudinally constrain the distal engagement portion relative to the catheter at the first engagement section, wherein the actuation member is distally advanceable within the actuator lumen to push the distal engagement portion out of the lumen, thereby releasing the distal engagement portion to permit expansion of the device from the collapsed configuration.

Clause 2. The system of Clause 1, wherein the distal engagement portion comprises an elongate body having a longitudinal axis that is substantially parallel or coaxial relative to the actuator lumen when the elongate body is positioned within the actuator lumen.

Clause 3. The system of Clause 2, wherein elongate body is comprises a wire.

Clause 4. The system of any of the previous clauses, wherein the device extends around a circumference of the catheter distal portion.

Clause 5. The system of any of the previous clauses, wherein the distal engagement portion radially overlaps with the distal coupling section when the distal engagement portion extends within the lumen.

Clause 6. The system of any of the previous clauses, wherein the distal engagement portion comprises a notch or a protrusion that radially engages with a notch or a protrusion of the distal coupling section when the distal engagement portion extends within the lumen to constrain axial motion of the distal engagement portion relative to the distal coupling section.

Clause 7. The system of any of the previous clauses, wherein one of the distal engagement portion and the distal coupling section comprises a notch, and the other one of the distal engagement portion and the distal coupling section comprises a protrusion, and wherein when the distal engagement portion is positioned within the lumen, the notch and the protrusion are coupled together to constrain axial motion of the distal engagement portion relative to the distal coupling section.

Clause 8. The system of Clause 7, wherein the distal engagement portion comprises the notch, and the distal coupling section comprises the protrusion, the notch and the protrusion being coupled together when the distal engagement portion is positioned within the lumen to constrain axial motion of the distal engagement portion relative to the distal coupling section.

Clause 9. The system of Clause 8, wherein the distal engagement portion comprises a wire, and wherein the actuation member comprises a wire.

Clause 10. The system of any of the previous clauses, wherein the distal engagement portion and the distal coupling section each comprise an interlocking tab, and wherein when the distal engagement portion is positioned within the lumen, the interlocking tabs are coupled together to constrain axial motion of the distal engagement portion relative to the distal coupling section.

Clause 11. The system of any of the previous clauses, wherein the distal engagement portion comprises a U-shaped body having a first portion coupled to the device and a free, second portion configured to extend into the lumen.

Clause 12. The system of any of the previous clauses, wherein the proximal engagement portion comprises a wire extending into the second actuator lumen, the proximal engagement portion having a longitudinal axis that is substantially parallel or coaxial to a longitudinal axis of the second actuator lumen.

Clause 13. The system of any of the previous clauses, wherein the actuator lumen comprises a diameter of between about 0.005″ and about 0.007″.

Clause 14. The system of any of the previous clauses, wherein the catheter further comprises a second actuator lumen, parallel to the actuator lumen, extending from the proximal portion to a second aperture at the second engagement section of the distal portion, and wherein the system further comprises a second elongate actuation member comprising a second distal coupling section movably disposed within the second lumen.

Clause 15. The system of Clause 14, wherein the second aperture comprises a perimeter defined by the intersection of an inner surface of the second lumen and a plane extending transversely through the second lumen.

Clause 16. The system of Clause 15, wherein the plane extends perpendicularly through the actuator lumen, and the second aperture is circular and opens toward the distal end of the catheter.

Clause 17. The system of any of Clauses 14-16, wherein the proximal engagement portion extends proximally through the second aperture into the second lumen toward the second distal coupling section to longitudinally constrain the proximal engagement portion relative to the catheter at the first engagement section.

Clause 18. The system of Clause 17, wherein the proximal engagement portion comprises a second elongate body having a longitudinal axis that is substantially parallel or coaxial relative to the second lumen when the second elongate body is positioned within the second lumen.

Clause 19. The system of any of Clauses 14-18, wherein the second lumen comprises a diameter of between about 0.005″ and about 0.007″.

Clause 20. The system of any of the previous clauses, wherein the first engagement section is positioned distal to the second engagement section.

Clause 21. The system of any of the previous clauses, wherein the first aperture is disposed at a distal end of the catheter.

Clause 22. The system of any of the previous clauses, wherein the first aperture comprises a perimeter defined by the intersection of an inner surface of the actuator lumen and a plane extending transversely through the actuator lumen.

Clause 23. The system of Clause 21, wherein the plane extends perpendicularly through the actuator lumen, and the first aperture is circular and opens toward the distal end of the catheter.

Clause 24. The system of any of the previous clauses, wherein the catheter further comprises a central lumen extending from the proximal portion to the distal portion, parallel to the actuator lumen.

Clause 25. The system of Clause 24, further comprising a hypotube, extending within the central lumen, the hypotube comprising a helical slot.

Clause 26. The system of Clause 25, wherein the helical slot comprises a variable pitch along the length of the hypotube.

Clause 27. The system of Clause 26, wherein the pitch increases toward the distal portion of the catheter.

Clause 28. The system of any of Clauses 24-27, wherein the central lumen comprises a diameter of between about 0.022″ and about 0.028″.

Clause 29. The system of any of the previous clauses, further comprising a handle component, coupled to the catheter and a proximal section of the actuation member, the handle component configured to move the actuation member longitudinally within the actuator lumen to cause release of the distal engagement portion.

Clause 30. A medical device delivery system, comprising: a catheter comprising proximal and distal portions and first and second lumens, the first lumen extending from the proximal portion to a first aperture at the distal portion, the second lumen extending from the proximal portion to a second aperture at the distal portion, the first aperture distal to the second aperture; a first elongate actuation member, longitudinally moveable within the first lumen, comprising a first distal coupling section; a second elongate actuation member, longitudinally moveable within the second lumen, comprising a second distal coupling section; and a medical device extending along an outer surface of the catheter distal portion in a collapsed configuration, the device having (i) a proximal engagement portion extending proximally through the second aperture into the second lumen and releasably coupled to the second actuation member, and (ii) a distal engagement portion extending proximally through the first aperture into the first lumen to releasably couple the distal engagement portion to the distal portion; wherein distal advancement of the first and second actuation members releases the proximal and distal engagement portions from the first and second lumens, thereby permitting expansion of the device from the collapsed configuration.

Clause 31. The system of Clause 30, wherein the first actuation member is distally advanceable within the first lumen to push the distal engagement portion out of the first lumen, thereby releasing the distal engagement portion to permit expansion of the device from the collapsed configuration.

Clause 32. The system of any of Clauses 30-31, wherein the distal engagement portion comprises an elongate body having a longitudinal axis that is substantially parallel or coaxial relative to the first lumen when the elongate body is positioned within the first lumen.

Clause 33. The system of any of Clauses 30-32, wherein the proximal engagement portion comprises an elongate body having a longitudinal axis that is substantially parallel or coaxial relative to the second lumen when the elongate body is positioned within the second lumen.

Clause 34. The system of any of Clauses 30-33, wherein the distal engagement portion radially overlaps with the first distal coupling section when the distal engagement portion extends within the first lumen.

Clause 35. The system of any of Clauses 30-35, wherein the distal engagement portion comprises a notch or a protrusion that radially engages with a notch or a protrusion of the first distal coupling section when the distal engagement portion extends within the first lumen to constrain axial motion of the distal engagement portion relative to the first distal coupling section.

Clause 36. The system of Clause 35, wherein the proximal engagement portion comprises a notch or a protrusion that radially engages with a notch or a protrusion of the second distal coupling section when the proximal engagement portion extends within the second lumen to constrain axial motion of the proximal engagement portion relative to the second distal coupling section.

Clause 37. The system of any of Clauses 30-36, wherein the distal engagement portion comprises a U-shaped body having a first portion coupled to the device and a free, second portion configured to extend into the first lumen.

Clause 38. A medical device delivery assembly, comprising: a catheter comprising a proximal portion, a distal portion, and an actuator lumen extending from the proximal portion to the distal portion and opening to a distal aperture; an actuation member comprising a proximal section and a distal section, the actuation member being longitudinally moveable within the actuator lumen; and a medical device having a distal engagement portion extending distally beyond the actuation member distal section and proximally into the lumen through the distal aperture, wherein (i) in a collapsed configuration, both the actuation member distal section and the device distal engagement portion extend within the lumen, and (ii) distal advancement of the actuation member within the lumen pushes the distal engagement portion out of the distal aperture to urge the device to an expanded configuration in which the device distal engagement portion is positioned outside of the lumen.

Clause 39. The assembly of Clause 38, wherein the medical device comprises a proximal engagement portion coupled relative to the catheter to constrain the proximal engagement portion.

Clause 40. The assembly of any of Clauses 38-39, wherein the device distal engagement portion extends longitudinally into the lumen through an aperture at a distal end of the lumen.

Clause 41. The assembly of any of Clauses 38-40, wherein the actuation member distal section radially overlaps with the device distal engagement portion in the collapsed configuration.

Clause 42. The assembly of Clause 41, wherein the device distal engagement portion comprises a notch that radially engages with a notch of the actuation member distal section in the collapsed configuration.

Clause 43. The assembly of any of Clauses 38-42, wherein one of the distal engagement portion and the distal section comprises a notch, and the other one of the distal engagement portion and the distal section comprises a protrusion, and wherein when the distal engagement portion is positioned within the lumen, the notch and the protrusion are coupled together to constrain longitudinal motion of the distal engagement portion relative to the distal section.

Clause 44. The assembly of Clause 43, wherein the distal engagement portion comprises the notch, and the distal section comprises the protrusion, the notch and the protrusion being coupled together when the distal engagement portion is positioned within the lumen to constrain longitudinal motion of the distal engagement portion relative to the distal section.

Clause 45. The assembly of Clause 44, wherein the device distal engagement portion comprises a wire, and wherein the actuation member comprises a wire.

Clause 46. The assembly of any of Clauses 38-45, wherein the device distal engagement portion and the actuation member distal section each comprise an interlocking tab, and wherein, in the collapsed configuration, the interlocking tabs are coupled together to constrain longitudinal motion of the distal engagement portion relative to the distal section.

Clause 47. The assembly of any of Clauses 38-46, wherein the device distal engagement portion comprises a U-shaped member having a first portion coupled to the device and a free, second portion configured to extend into the lumen.

Clause 48. The assembly of Clause 47, wherein the first and second portions of the U-shaped member are joined together at a bend and the first and second portions extend substantially parallel relative to each other.

Clause 49. The assembly of any of Clauses 38-48, wherein the catheter further comprises a second actuator lumen, parallel to the actuator lumen, and the assembly further comprises a second actuation member, the second actuation member being longitudinally moveable within the second actuator lumen and comprising a distal section that is engageable with a proximal engagement portion of the medical device to constrain the proximal engagement portion within the second actuator lumen.

Clause 50. The assembly of Clause 49, wherein the actuation member distal end extends to a first engagement section and the second actuation member distal end extends to a second engagement section, wherein the medical device is engageable to the catheter at the first and second engagement sections.

Clause 51. The assembly of Clause 50, wherein the first engagement section is positioned distal of the second engagement section.

Clause 52. The assembly of Clause 51, wherein the second engagement section comprises a second aperture extending from the second actuator lumen to an outer surface of the catheter.

Clause 53. The assembly of Clause 52, wherein the second aperture comprises a perimeter defined by the intersection of an inner surface of the second actuator lumen and a plane extending transversely through the second actuator lumen.

Clause 54. The assembly of Clause 53, wherein the plane extends perpendicularly through the second actuator lumen, and the second aperture comprises a circle opening toward the distal end of the catheter.

Clause 55. The assembly of any of Clauses 38-54, wherein the device engagement portion comprises a proximal component of the device.

Clause 56. The assembly of any of Clauses 38-55, wherein the device engagement portion comprises a distal component of the device.

Clause 57. The assembly of any of Clauses 38-56, wherein the catheter extends within a lumen of the device when the device is in the collapsed configuration.

Clause 58. The assembly of any of Clauses 38-57, further comprising a handle component coupled to the actuation member proximal section, the handle component configured to move the actuation member longitudinally within the actuator lumen.

Clause 59. A medical device delivery assembly, comprising: a catheter comprising a proximal portion, a distal portion, and an actuator lumen extending from the proximal portion to the distal portion and opening to an actuator lumen aperture; and an actuation member comprising a distal section, the actuation member being longitudinally moveable within the actuator lumen, the distal section of the actuation member comprising an engagement member for engaging a proximal portion of a medical device extending within the actuator lumen aperture such that the engagement member and the medical device proximal portion (i) maintain substantially fixed longitudinal positions relative to each other when the medical device proximal portion is positioned longitudinally within the actuator lumen and (ii) are longitudinally movable together within the actuator lumen as a unit between a first position in which the medical device is engaged with the engagement member and a second position in which the medical device is disengaged from the engagement member.

Clause 60. The assembly of Clause 59, further comprising a medical device, the medical device having collapsed and expanded configurations, wherein (i) in the collapsed configuration, both the engagement member and the medical device proximal portion radially overlap at a first position within the actuator lumen, and (ii) in the expanded configuration, the engagement member is positioned at a second position, longitudinally spaced apart from the first position, and the medical device proximal portion is positioned outside of the actuator lumen.

Clause 61. The assembly of Clause 60, wherein one of the engagement member and the medical device proximal portion comprises a notch, and the other one of the engagement member and the medical device proximal portion comprises a protrusion, and wherein when the medical device proximal portion is positioned within the actuator lumen, the notch and the protrusion are coupled together to constrain longitudinal motion of the engagement member relative to the medical device proximal portion.

Clause 62. The assembly of Clause 61, wherein the medical device comprises an annular protrusion, and wherein the engagement member comprises a socket and a distal stop member, the socket being configured to receive the annular protrusion, the distal stop member being configured to longitudinally constrain the annular protrusion within the socket such that when the annular protrusion is disposed within both the socket and the actuator lumen, the annular protrusion is (i) radially constrained by the actuator lumen and the engagement member within the socket and (ii) longitudinally constrained by the distal stop member within the socket and relative to the engagement member.

Clause 63. The assembly of any of Clauses 60-62, wherein the medical device proximal portion and the engagement member each comprise an interlocking tab, and wherein, in the collapsed configuration, the interlocking tabs are coupled together to constrain longitudinal motion of the medical device proximal portion relative to the engagement member.

Clause 64. The assembly of any of Clauses 60-63, wherein the medical device proximal portion comprises a wire.

Clause 65. The assembly of any of Clauses 60-64, wherein the catheter extends within a lumen of the device when the device is in the collapsed configuration.

Clause 66. The assembly of any of Clauses 60-65, wherein the medical device is entirely disposed within the actuator lumen when the device is in the collapsed configuration.

Clause 67. The assembly of any of Clauses 60-66, wherein the medical device comprises frame having a pair of loops interconnected by a link portion, and a cover component having a pair of expandable portions that each overlap a respective loop of the pair of loops, the cover component comprising a non-expandable portion interposed between the expandable portions, the non-expandable portion overlapping the link portion, the medical device being expandable such that the expandable portions expand with the pair of loops and the non-expandable portion remains in an unexpanded diameter.

Clause 68. The assembly of any of Clauses 60-67, wherein the medical device comprises a distalmost loop, the medical device being received within the actuator lumen of the catheter, wherein the distalmost loop extends out of the actuator lumen and a distal section of the catheter extends through the distalmost loop.

Clause 69. The assembly of any of Clauses 59-68, wherein the engagement member comprises a notch for radially engaging the medical device.

Clause 70. The assembly of any of Clauses 59-69, wherein the actuation member comprises a wire.

Clause 71. The assembly of any of Clauses 59-70, further comprising a guidewire or fluid lumen extending longitudinally from the catheter proximal portion to the catheter distal portion and having a guidewire aperture.

Clause 72. The assembly of Clause 71, wherein the guidewire aperture is positioned distal to the actuator lumen aperture.

Clause 73. The assembly of any of Clauses 71-72, wherein the catheter distal portion comprises first and second sections, the first section having a first cross-section, the second section having a second cross-section, the first section being (i) proximal of the second section and (ii) having a first cross-section with a profile larger than a profile of a second cross-section.

Clause 74. The assembly of Clause 73, wherein a cross-section of the first section comprises both the actuator lumen and the guidewire lumen, and a cross-section of the second section comprises the guidewire lumen but does not comprise the actuator lumen.

Clause 75. The assembly of Clause 74, wherein the actuator lumen aperture is positioned at a distal end of the first section and opens to the second section.

Clause 76. The assembly of any of Clauses 74-75, wherein the actuator lumen aperture is positioned at a distal end of the second section.

Clause 77. The assembly of any of Clauses 59-76, comprising any medical device disclosed herein.

Clause 78. The assembly of any of Clauses 59-77, wherein the catheter further comprises a second actuator lumen, parallel to the actuator lumen, and the assembly further comprises a second actuation member, the second actuation member being longitudinally moveable within the second actuator lumen.

Clause 79. The assembly of Clause 78, wherein the second actuation member comprises a distal section that is engageable with a distal portion of a medical device to constrain the distal portion within the second actuator lumen.

Clause 80. The assembly of any of Clauses 78-79, wherein the second actuator lumen extends to a second actuator lumen aperture, the second actuator lumen aperture being positioned distal of the first actuator lumen aperture.

Clause 81. The assembly of Clause 80, wherein the actuator lumen aperture comprises a perimeter defined by the intersection of an inner surface of the actuator lumen and a plane extending transversely through the actuator lumen.

Clause 82. The assembly of Clause 81, wherein the plane extends perpendicularly through the second actuator lumen, and the second aperture comprises a circle opening toward the distal end of the catheter.

Clause 83. The assembly of any of Clauses 59-82, further comprising a handle component coupled to a proximal section of the actuation member, the handle component configured to move the actuation member longitudinally within the actuator lumen.

Clause 84. A medical catheter, comprising: an elongate body having proximal and distal portions and first and second engagement sections at the distal portion; a first lumen extending from the proximal portion to a first aperture at a distal end of the elongate body; and a second lumen extending from the proximal portion to a second aperture, proximal to the first aperture, the second aperture opening toward the distal end.

Clause 85. The catheter of Clause 84, further comprising a first tubular member extending within the first lumen.

Clause 86. The catheter of Clause 85, wherein the first lumen comprises an inner diameter of between about 0.005″ and about 0.007″.

Clause 87. The catheter of any of Clauses 84-86, further comprising a second tubular member extending within the second lumen.

Clause 88. The catheter of Clause 87, wherein the second lumen comprises an inner diameter of between about 0.005″ and about 0.007″.

Clause 89. The catheter of any of Clauses 84-88, further comprising a central lumen extending within the body, parallel to the first and second lumens.

Clause 90. The catheter of Clause 89, further comprising a hypotube, extending within the central lumen, the hypotube comprising a helical slot.

Clause 91. The catheter of Clause 90, wherein the helical slot comprises a pitch that varies along the length of the hypotube.

Clause 92. The catheter of Clause 91, wherein the pitch increases toward the distal portion of the catheter.

Clause 93. The catheter of Clause 92, wherein the hypotube comprises at least one section having a constant pitch.

Clause 94. The catheter of any of Clauses 92-93, wherein the helical slot comprises first and second sections, the first section having a first pitch, and the second section having a second pitch, greater than the first pitch.

Clause 95. The catheter of Clause 94, wherein the first and second sections each have a length of between about 10 inches and about 14 inches.

Clause 96. The catheter of any of Clauses 94-95, wherein the first and second sections each have a length of between about 11 inches and about 13 inches.

Clause 97. The catheter of any of Clauses 94-96, wherein the first and second sections each have a length of about 12 inches.

Clause 98. A method of assembling an implant delivery system, comprising: providing a catheter, first and second actuation members, and an implant, the catheter having proximal and distal sections and first and second lumens, the first lumen extending from the proximal section and terminating at a first aperture in the distal section, the second lumen extending from the proximal section and terminating at a second aperture in the distal section, the implant having first and second engagement members; positioning the implant over the catheter; proximally inserting the first engagement member through the first aperture to couple the first elongate portion with the catheter; longitudinally stretching the implant over the catheter to position the second engagement member adjacent to the second engagement section; and inserting the second engagement member through the second aperture to couple the second elongate portion with the catheter.

Clause 99. The method of Clause 98, wherein the positioning comprises inserting the catheter into the implant.

Clause 100. The method of any of Clauses 98-99, wherein the first engagement member comprises a first elongate member having a first longitudinal axis, and the inserting the first engagement member comprises aligning the first longitudinal axis with a longitudinal axis of the first lumen during the inserting.

Clause 101. The method of Clause 100, further comprising coupling the first elongate member to the first distal coupling portion prior to inserting the first elongate member within the first lumen.

Clause 102. The method of Clause 101, wherein the coupling the first elongate member comprises radially overlapping the first elongate member with the first distal coupling portion.

Clause 103. The method of Clause 102, wherein the overlapping comprises fitting a notch or protrusion of the first elongate member into a notch or protrusion of the first distal coupling portion.

Clause 104. The method of any of Clauses 98-103, wherein the second engagement member comprises a second elongate member having a second longitudinal axis, and the inserting the second engagement member comprises aligning the second longitudinal axis with a longitudinal axis of the second lumen during the inserting.

Clause 105. The method of Clause 104, further comprising coupling the second elongate member to the second distal coupling portion prior to inserting the second elongate member within the second lumen.

Clause 106. The method of Clause 105, wherein the coupling the second elongate member comprises radially overlapping the second elongate member with the second distal coupling portion.

Clause 107. The method of Clause 106, wherein the overlapping comprises fitting a notch or protrusion of the second elongate member into a notch or protrusion of the second distal coupling portion.

Clause 108. A method of operating an implant delivery system, comprising: providing a catheter, a first actuation member, and a self-expanding implant, the catheter having proximal and distal sections and a first lumen extending from the proximal section and terminating at a first aperture in the distal section, the implant having a first engagement member extending proximally into the first aperture to be coupled to the catheter; advancing the catheter into a lumen of the body; and longitudinally moving the first actuation member within the first lumen to longitudinally move the first engagement portion, thereby urging the first engagement portion out of the first lumen and permitting the implant to expand from a collapsed state.

Clause 109. The method of Clause 108, wherein the longitudinally moving the first actuation member comprises distally advancing the first actuation member within the first lumen to push the first engagement portion distally out of the first lumen.

Clause 110. The method of any of Clauses 108-109, wherein the catheter comprises a second actuation member extending from the proximal section and terminating at a second aperture in the distal section, the implant having a second engagement member extending proximally into the second aperture be coupled to the second actuation member within the catheter.

Clause 111. The method of Clause 110, further comprising longitudinally moving the second actuation member within the second lumen to longitudinally move the second engagement portion, thereby urging the second engagement portion out of the second lumen and releasing the implant from the catheter.

Clause 112. The method of Clause 111, wherein the longitudinally moving the second actuation member comprises distally advancing the second actuation member within the second lumen to push the second engagement portion distally out of the second lumen.

Clause 113. The method of any of Clauses 111-112, wherein the longitudinally moving the first actuation member is performed prior to longitudinally moving the second actuation member.

Clause 114. The method of any of Clauses 111-113, wherein prior to longitudinally moving the second actuation member, the method comprises injecting a material.

Clause 115. The method of Clause 114, wherein the implant comprises a distal aperture and wherein the material is injected through the distal aperture.

Clause 116. The method of any of Clauses 114-115, wherein after the material is injected and after longitudinally moving the second actuation member, the method further comprises proximally withdrawing the catheter such that a distal component of the catheter moves to a closed position to close the aperture.

Clause 117. The method of any of Clauses 108-116, further comprising proximally retracting the catheter into a delivery catheter to cause the device to collapse back toward the collapsed state.

Clause 118. A medical device, comprising: a tubular cover member having an open proximal end portion and a distal end portion, the distal end portion comprising an aperture extending therethrough; and a frame, supporting the cover member, comprising proximal and distal components that are coupled to the respective proximal and distal end portions of the cover member, the distal component having an open configuration in which the cover member aperture is open and a closed configuration in which the distal component folds onto the cover member such that the aperture is closed, the distal component being biased toward the closed position.

Clause 119. The device of Clause 118, wherein the distal component comprises a hoop that is coupled to the distal end portion of the tubular member such that the aperture is bounded by the hoop.

Clause 120. The device of any of Clauses 118-119, wherein in the open configuration, a longitudinal axis of the device extends at an angle of between about 30 degrees and about 90 degrees relative to a hoop plane through which the hoop passes.

Clause 121. The device of Clause 120, wherein in the closed configuration, the longitudinal axis extends at an angle of between about 0 degrees and about 10 degrees relative to the hoop plane.

Clause 122. The device of any of Clauses 118-121, wherein the frame comprises a plurality of hoops.

Clause 123. The device of any of Clauses 118-122, wherein the frame comprises a pair of wires.

Clause 124. The device of Clause 123, wherein the pair of wires are coupled together in a first backbone portion and spaced apart from each other in a hoop section, the hoop section configured with the wires diverging from each other at the first backbone portion and converging toward each other to be coupled together in a second backbone portion.

Clause 125. The device of Clause 123, wherein the pair of wires are welded together in backbone portions of the frame.

Clause 126. The device of any of Clauses 118-125, wherein the frame is positioned inside the cover member.

Clause 127. A medical device frame for implantation into a body lumen, the frame comprising proximal and distal engagement portions and an expandable central portion disposed between the proximal and distal engagement portions, the proximal engagement portion comprising an elongate proximal coupling member having a radial notch, the distal engagement portion comprising an elongate distal coupling member having a radial notch, the distal engagement portion having a bend such that the distal coupling member extends from the distal engagement portion in a proximal direction.

Clause 128. The device frame of Clause 127, wherein the frame comprises a pair of wires diverging away from each other and converging toward each other to form a series of expandable components.

Clause 129. The device frame of Clause 128, wherein the series of expandable components comprises a distal hoop.

Clause 130. The device frame of Clause 129, wherein in an open configuration, a longitudinal axis of the device frame extends at an angle of between about 30 degrees and about 90 degrees relative to a hoop plane through which the hoop passes.

Clause 131. The device frame of Clause 130, wherein in a closed configuration, the longitudinal axis extends at an angle of between about 0 degrees and about 10 degrees relative to the hoop plane.

Clause 132. The device frame of any of Clauses 128-131, wherein the frame comprises a plurality of hoops.

Clause 133. The device frame of any of Clauses 128-132, wherein the pair of wires are coupled together in a first backbone portion and spaced apart from each other in a hoop section, the hoop section configured with the wires diverging from each other at the first backbone portion and converging toward each other to be coupled together in a second backbone portion.

Clause 134. The device frame of Clause 133, wherein the pair of wires are welded together in backbone portions of the frame.

Clause 135. The device frame of any of Clauses 127-134, wherein the central portion comprises plurality of hoops interconnected by longitudinal backbones.

Clause 136. The device frame of any of Clauses 127-135, further comprising a tubular cover member having an open proximal end portion and a distal end portion, the distal end portion comprising an aperture extending therethrough, the cover member coupled to the frame.

Clause 137. The device frame of Clause 136, wherein the frame is positioned inside the cover member.

Clause 138. A medical device frame for implantation into a body lumen, the frame comprising a proximal portion, a distal portion, and an expandable central portion disposed between the proximal and distal portions, the proximal portion comprising an elongate proximal coupling member, the proximal coupling member comprising a radial protrusion and a recessed portion for overlapping and radially engaging a corresponding engagement member of a delivery device, the expandable central portion comprising first and second loops that are interconnected by a backbone portion extending between the first and second loops, wherein interconnections between the backbone portion and the first and second loops bias the loops from a collapsed, substantially linear configuration to an expanded configuration in which the first and second loops extend transversely or substantially parallel relative to each other.

Clause 139. The device frame of Clause 138, wherein the distal portion comprises an elongate distal coupling member having a radial notch.

Clause 140. The device frame of Clause 139, wherein the distal portion comprises a bend such that the distal coupling member extends from the distal engagement portion in a proximal direction.

Clause 141. The device frame of any of Clauses 138-140, wherein the frame comprises a pair of wires diverging away from each other and converging toward each other to form the first and second loops.

Clause 142. The device frame of Clause 141, wherein the pair of wires are welded together along the backbone portion of the frame.

Clause 143. The device frame of any of Clauses 141-142, wherein the pair of wires (i) converge at the interconnections between the backbone portion and the first and second loops and (ii) diverge between the interconnections along a longitudinal extent of the backbone portion such that the backbone portion comprises the wires in a spaced-apart configuration.

Clause 144. The device frame of Clause 143, wherein each of the wires extends in a semi-circular arc along the backbone portion.

Clause 145. The device frame of any of Clauses 138-144, wherein the distal portion comprises a distal loop.

Clause 146. The device frame of any of Clauses 138-145, wherein in an open configuration, planes through which the first and second loops pass extend at angles of between about 30 degrees and about 90 degrees relative to a longitudinal axis of the device frame.

Clause 147. The device frame of any of Clauses 138-146, wherein in a closed configuration, planes through which the first and second loops pass extend at angles of between about 0 degrees and about 10 degrees relative to a longitudinal axis of the device frame.

Clause 148. The device frame of any of Clauses 138-146, further comprising a tubular cover member coupled to the frame.

Clause 149. The device frame of Clause 148, wherein the frame is positioned inside the cover member.

Clause 150. A medical device for implantation into a body lumen, the device comprising a frame and a cover component, the frame comprising first and second expandable loops that are interconnected by a backbone portion extending between the first and second loops, wherein interconnections between the backbone portion and the first and second loops bias the loops from a collapsed, substantially linear configuration to an expanded configuration in which the first and second loops extend transversely relative to the backbone portion and substantially parallel relative to each other, the first and second loops having expanded diameters in the expanded configuration, the cover component comprising a substantially tubular member having a collapsed diameter when surrounding the frame in the collapsed configuration, the cover component comprising expandable portions longitudinally aligned with the first and second loops in the collapsed configuration and a substantially non-expandable portion longitudinally interposed along between the first and second loops along the backbone portion, wherein in the expanded configuration, the expandable portions expand from the collapsed diameter to the expanded diameters of the first and second loops and the substantially non-expandable portion remains in the collapsed diameter.

Clause 151. The device of Clause 150, wherein the frame comprises a proximal coupling member for engaging with a portion of a delivery device.

Clause 152. The device of any of Clauses 150-151, wherein the frame comprises a pair of wires diverging away from each other and converging toward each other to form the first and second loops.

Clause 153. The device of Clause 152, wherein the pair of wires are welded together along the backbone portion of the frame.

Clause 154. The device of any of Clauses 152-153, wherein the pair of wires (i) converge at the interconnections between the backbone portion and the first and second loops and (ii) diverge between the interconnections along a longitudinal extent of the backbone portion such that the backbone portion comprises the wires in a spaced-apart configuration.

Clause 155. The device of any of Clauses 152-154, wherein each of the wires extends in a semi-circular arc along the backbone portion.

Clause 156. The device of any of Clauses 150-155, wherein expanded diameters of the expandable portions are between about 6 times and about 30 times the collapsed diameter.

Clause 157. The device of any of Clauses 150-156, wherein expandable portions comprise pre-stretched sections of the cover component.

Clause 158. The device of any of Clauses 150-157, wherein expandable portions comprise additional folds of material to facilitate expansion to the expanded diameters.

Clause 159. An expandable device for delivery to a target location in a body vessel, comprising: a helical member having a proximal end portion, a distal end portion, and a first lumen extending between the proximal and distal end portions; and an occlusive member having a proximal end portion coupled to helical member distal end portion, the occlusive member having a plurality of support components radially expandable from a collapsed configuration to an expanded configuration.

Clause 160. The device of Clause 159, wherein the support components are interconnected at the occlusive member proximal end portion.

Clause 161. The device of any of Clauses 159-160, further comprising a coupling member interconnecting the helical member and the occlusive member.

Clause 162. The device of Clause 161, wherein the support components are coupled to the coupling member, the support components being deflectable between the collapsed and expanded configurations.

Clause 163. The device of any of Clauses 159-162, wherein the support components are coupled to the helical member, the support components being deflectable between the collapsed and expanded configurations.

Clause 164. The device of any of Clauses 159-163, wherein the support components comprise first and second portions, the first portion being deflectably coupled to the coupling member, the second portion being deflectably coupled to the first portion to move from the collapsed configuration to the expanded configuration.

Clause 165. The device of Clause 164, wherein a proximal end of the second portion is deflectably coupled to a distal end of the first portion.

Clause 166. The device of Clause 164, wherein a proximal end of the second portion is pivotably coupled to a distal end of the first portion.

Clause 167. The device of Clause 164, wherein the first portion comprises a plurality of elongate members.

Clause 168. The device of Clause 167, wherein the plurality of elongate members each comprise a looped wire.

Clause 169. The device of Clause 164, wherein the second portion comprises a wire formed in a loop.

Clause 170. The device of Clause 169, wherein the wire comprises a plurality of peak sections and valley sections, and wherein the first portion distal end is coupled to the valley sections.

Clause 171. The device of Clause 170, wherein the first portion comprises a plurality of elongate members coupled to the valley sections.

Clause 172. The device of any of Clauses 159-163, further comprising a plurality of linking portions, each linking portion extending between adjacent support components.

Clause 173. The device of Clause 172, wherein the support portions each comprise elongate wires extending axially in a direction away from the helical member.

Clause 174. The device of any of Clauses 159-173, wherein each of the support components comprises a wire formed a loop.

Clause 175. The device of Clause 174, wherein the wire comprises a loop having a distal bend forming opposing loop halves and opposing lateral bends in the opposing loop halves, the wire being configured to resiliently compress toward the collapsed configuration and resiliently release to the expanded configuration.

Clause 176. The device of Clause 175, wherein the distal bend comprises a substantially 45° angle.

Clause 177. The device of Clause 175, wherein the opposing lateral bends comprise an obtuse angle.

Clause 178. The device of Clause 175, wherein the wire further comprises a radial bend, wherein a distal portion of the wire bends away from a longitudinal axis of the occlusive member at the radial bend.

Clause 179. The device of any of Clauses 159-178, wherein the occlusive member comprises a pair of wires, wherein in the collapsed configuration, the wires are substantially linearly arranged and in the expanded configuration, the wires are separated to form the plurality of support components.

Clause 180. The device of Clause 179, wherein the pair of wires have substantially mirror configurations.

Clause 181. The device of any of Clauses 179-180, wherein the plurality of support components are substantially circular.

Clause 182. The device of any of Clauses 179-181, wherein the support components are spaced apart along a longitudinal axis of the occlusive member.

Clause 183. The device of Clause 182, wherein the support components comprise distal and proximal components and at least one intermediate component positioned longitudinally between the distal and proximal components.

Clause 184. The device of Clause 183, wherein the distal and proximal components comprise an expanded dimension that is greater than an expanded dimension of the at least one intermediate component.

Clause 185. The device of Clause 183, wherein the distal component comprises an expanded dimension that is substantially equal to an expanded dimension of the proximal component.

Clause 186. The device of any of Clauses 183-185, wherein the at least one intermediate component comprises two intermediate components having different expanded dimensions.

Clause 187. The device of Clause 186, wherein the expanded dimensions of the two intermediate components are different than the expanded dimensions of the proximal and distal components.

Clause 188. The device of Clause 184, wherein the expanded dimensions of the two intermediate components is less than the expanded dimensions of the proximal and distal components.

Clause 189. The device of any of Clauses 159-188, wherein the helical member comprises a flat coil following a helical path.

Clause 190. The device of any of Clauses 159-189, wherein the helical member comprises a substantially rectangular cross-sectional shape.

Clause 191. The device of any of Clauses 159-190, wherein an expandable portion of the helical member is entirely axially spaced apart from an expandable portion of the occlusive member.

Clause 192. The device of any of Clauses 159-191, wherein the device comprises a cover member attached to the device.

Clause 193. The device of Clause 192, wherein the cover member extends along the occlusive member.

Clause 194. The device of Clause 193, wherein the occlusive member is positioned within the cover member.

Clause 195. The device of any of Clauses 193-194, wherein the cover is coupled to a coupling member that interconnects the helical member and the occlusive member.

Clause 196. The device of any of Clauses 159-195, wherein the helical member comprises an axial cross-sectional width and a radial cross-sectional thickness, wherein the axial cross-sectional width is greater than the radial cross-sectional thickness.

Clause 197. A method of implanting an intraluminal device, comprising: advancing a catheter to a target location within a body vessel, the catheter having a lumen in which the device is disposed, the device comprising a helical member and an occlusive member coupled to a distal end of the helical member, the occlusive member having a plurality of support components radially expandable from a collapsed configuration to an expanded configuration; and advancing the device out of the lumen to permit separate expansion of the helical member and the occlusive member.

Clause 198. The method of Clause 197, wherein the advancing comprises permitting a cover member to expand along the occlusive member.

Clause 199. The method of Clause 198, wherein the advancing comprises positioning the cover member over the occlusive member.

Clause 200. The method of any of Clauses 197-198, wherein the advancing comprises permitting expansion of the helical member prior to expansion of the occlusive member.

Clause 201. An implant frame comprising any of the features disclosed in any of Clauses 1-200.

Clause 202. An implant, as recited in any of Clauses 1-201, further comprising a distal or medial valve component.

Clause 203. An implant comprising any of the features disclosed in any of Clauses 1-202.

Clause 204. An catheter comprising any of the features disclosed in any of Clauses 1-203.

Clause 205. An implant delivery system comprising any of the features disclosed in any of Clauses 1-204.

Clause 206. An implant delivery system comprising any of the features disclosed in any of Clauses 1-205, wherein the system is configured to be advanced over a guidewire to a target region within a body lumen.

Clause 207. A method of operating any of the frames, implants, catheters, assemblies, or systems disclosed in any of Clauses 1-206.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1A is a perspective view of an implant carrier assembly, according to some embodiments.

FIG. 1B illustrates a perspective view of another implant carrier assembly, according to some embodiments.

FIG. 2 is a perspective view of an implant carrier assembly and implant carried thereon, according to some embodiments.

FIG. 3 shows a perspective view of an implant support frame in an expanded state, according to some embodiments.

FIG. 4A shows an end view of the implant support frame of FIG. 3, according to some embodiments.

FIG. 4B shows a side view of the implant support frame of FIG. 3, according to some embodiments.

FIG. 4C shows a top view of the implant support frame of FIG. 3, according to some embodiments.

FIG. 5A is a perspective view of a distal portion of a catheter of the assembly of FIG. 2, according to some embodiments.

FIG. 5B is an end view of the catheter of FIG. 5A.

FIG. 5C is a perspective view of the catheter of FIG. 5A wherein the luminal inserts have been removed, according to some embodiments.

FIG. 5D is a cross-sectional top view of the catheter of FIG. 5B, taken along section lines 5D-5D of FIG. 5B.

FIG. 6A is a perspective view of a cross-section of the catheter of the assembly of FIG. 2, taken along section lines 6A-6A of FIG. 5D, according to some embodiments.

FIG. 6B is an end view of the cross section of the catheter of FIG. 6A.

FIG. 6C is a perspective view of the cross section of the catheter of FIG. 6A wherein the luminal inserts have been removed, according to some embodiments.

FIG. 6D is a cross-sectional top view of an alternative embodiment of a catheter, according to some embodiments.

FIG. 7A is an enlarged perspective view of a proximal section of an implant engaged with a catheter of the assembly of FIG. 2, according to some embodiments.

FIG. 7B is a cross-sectional perspective view of the implant and catheter illustrated in FIG. 7A.

FIG. 8A is an enlarged perspective view of a distal section of an implant engaged with the catheter of the assembly of FIG. 2, according to some embodiments.

FIG. 8B is a cross-sectional perspective view of the implant and catheter illustrated in FIG. 8A.

FIG. 9 is a side view of a guidewire lumen insert, according to some embodiments.

FIG. 10 is an enlarged side view of the guidewire lumen insert of FIG. 9.

FIGS. 11A-11C are schematic illustrations of stages in which the implant carrier assembly of FIG. 2 expands and collapses an implant carried thereon within a body lumen, according to some embodiments.

FIGS. 12A-12C are schematic illustrations of stages in which the implant carrier assembly of FIG. 2 releases an implant into a body lumen, releasing a distal end of the implant before releasing a proximal end of the implant, according to some embodiments.

FIGS. 13A-13C are schematic illustrations of stages in which the implant carrier assembly of FIG. 2 releases an implant into a body lumen, releasing a proximal end of the implant before releasing a distal end of the implant, according to some embodiments.

FIG. 14 is a perspective view of another implant carrier assembly and implant carried thereon, according to some embodiments.

FIG. 15 is an enlarged perspective view of the implant carrier assembly of FIG. 14.

FIG. 16 is a side view of the implant carrier assembly and implant of FIG. 14, illustrating engagement between an elongate actuator member and the implant, according to some embodiments.

FIG. 17 is an end view of the implant carrier assembly of FIG. 14.

FIGS. 18A-18D are schematic illustrations of stages in which the implant carrier assembly of FIG. 14 releases an implant into a body lumen, according to some embodiments.

FIG. 19 is a side view of an implant carrier assembly wherein the implant is radially constrained within a catheter, according to some embodiments.

FIG. 20 is a perspective view of yet another implant carrier assembly and an implant carried thereon, according to some embodiments.

FIG. 21 is an enlarged perspective view of a distal portion of the implant carrier assembly and implant of FIG. 20.

FIG. 22 is a side view implant carrier assembly and implant of FIG. 20.

FIGS. 23A-23E are schematic illustrations of stages in which an implant carrier assembly releases an implant, according to some embodiments.

FIG. 24 is a perspective view of a proximal engagement portion of the implant of FIG. 20 and a distal engagement portion of an actuation member, according to some embodiments.

FIG. 25A is a perspective view of an implant device in an expanded state, according to some embodiments.

FIG. 25B is a perspective view of the implant device of FIG. 20 in a collapsed state, according to some embodiments.

FIG. 26 is a perspective view of another implant in an expanded state, according to some embodiments.

FIG. 27 is a perspective view of an implant or medical device having a cover member and a distal valve mechanism, according to some embodiments.

FIGS. 28A-28B are schematic illustrations of stages in which the implant carrier releases the implant of FIG. 27 into a body lumen, according to some embodiments.

FIG. 29 is a perspective view of another implant having a cover member and a medial valve mechanism, according to some embodiments.

FIGS. 30A-30B are schematic illustrations of stages in which the implant carrier releases the implant of FIG. 29 into a body lumen, according to some embodiments.

FIG. 31 is a side view of an implant carrier assembly having an implant carried thereon, wherein the implant comprises a cover member having at least one fold for supporting a hydrogel therein, according to some embodiments.

FIG. 32 is an enlarged side view of the fold of the cover member of FIG. 31.

FIGS. 33A-33B are side views of configurations of a cover member positioned about an implant frame, according to some embodiments.

FIG. 34 is a side view of a cover member, according to some embodiments.

FIGS. 35A-35E illustrate steps in a process for manufacturing an implant using the cover member of FIG. 34, according to some embodiments.

FIGS. 36-38 show perspective views of an occlusive implant, according to some embodiments.

FIGS. 39-41 show perspective view of other occlusive implants, according to some embodiments.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

While the present description sets forth specific details of various embodiments, it will be appreciated that the description is illustrative only and should not be construed in any way as limiting. It is contemplated that although particular embodiments of the present inventions may be disclosed or shown in particular contexts, such embodiments can be used in a variety of endoluminal applications. Various applications of such embodiments and modifications thereto, which may occur to those who are skilled in the art, are also encompassed by the general concepts described herein.

The present disclosure provides various embodiments of an expandable device, such as a stent, and a catheter for supporting and delivering the stent, as well as methods of using the devices and catheters.

According to some embodiments, devices, catheters, systems, and methods disclosed herein can be used for percutaneous, peripheral occlusion of the arterial and venous vasculature. For example, some embodiments can be used to treat pelvic venous incompetence, varicocele, gonadal vein for pelvic varices in females with chronic pelvic pain, stop blood loss from a damaged blood vessel due to a traumatic arterial injury, stop hemorrhage caused by a neoplasia, and close an abnormal blood vessel or blood vessels supplying a vascular anomaly such as arteriovenous malformations or arteriovenous fistulas, and other conditions.

According to some embodiments, devices, catheters, systems, and methods disclosed herein can also be used for percutaneous, peripheral stenting of the arterial and venous vasculature.

According to some embodiments, an assembly can be provided including an implant or medical device and a catheter, which can be configured to engage, support, and/or house the device for delivery to a treatment location. The implant can be self-expanding. The device can be engaged, supported, and/or housed along a distal portion of the device. Some embodiments can advantageously provide an assembly that has a cross-sectional profile that is much less than existing medical implant delivery assemblies.

For example, the catheter can define an outer diameter from about 2 Fr to about 12 Fr, as noted in Table 1 below and discussed further herein. These dimensions are provided for illustrative purposes only, and the sizes of the components disclosed herein can vary from those sizes listed below.

TABLE 1

| French | Diameter | Diameter |
|---|---|---|
| Gauge | (mm) | (inches) |
| 2 | 0.67 | 0.025 |
| 3 | 1 | 0.039 |
| 4 | 1.33 | 0.053 |
| 5 | 1.67 | 0.066 |
| 6 | 2 | 0.079 |
| 7 | 2.3 | 0.092 |
| 8 | 2.7 | 0.105 |
| 9 | 3 | 0.118 |
| 10 | 3.3 | 0.131 |
| 11 | 3.7 | 0.144 |
| 12 | 4 | 0.158 |

According to some embodiments, the reduced diameter or reduced cross-sectional profile can be achieved by using stent or frame structures that can have a nominal profile that is less than about five or ten times the cross-sectional profile of the filament(s) or wire forming the stent or frame structure. For example, in some embodiments, the stent or frame structure can be formed using a single elongate wire that is drawn into a generally linear configuration and moved through a catheter lumen toward the target site. Some embodiments can comprise two or more elongate wires that can be drawn into generally elongate linear configurations. Accordingly, various embodiments can be provided in which the elongate wires are drawn into a minimum profile configuration that allows the stent to assume a collapsed configuration having a cross-sectional profile that allows the stent to be loaded and delivered using a very small gauge catheter.

In accordance with some embodiments, a medical implant can be provided that can be used in a variety of clinical applications, such as vessel occlusion, stenting, or other functions within a body vessel. The medical implant can comprise a frame and one or more secondary components.

As noted, in some embodiments, the implant can at least partially occlude or block flow in a body lumen, such as a blood vessel. Some embodiments can be configured to provide complete and immediate occlusion of target lumen. Further, some embodiments can be configured to prevent or reduce any tendency for migration of the deployed device under pulsatile blood pressure. Furthermore, some embodiments can be configured to facilitate precise and well controlled deployment of the device for structure with movement of the device in and out of the catheter up until the moment of final detachment.

The frame can comprise one or more resilient members, such as wires, which can be drawn out into a delivery configuration in which the frame is in a generally linear configuration and thereafter expand to an expanded state when released from a delivery device, such as a catheter.

Various embodiments of the frame can be comprise one or more features, such as having a variable pitch, an alternating pitch, a consistent pitch, a dual wire loop configuration, and/or other features disclosed herein. Further, embodiments of the frame can be used with occlusive structures, valves, occlusive covers, fibrous membranes, and the like.

Further, in accordance with some embodiments, the implants and delivery systems can be used in combination with image-guided placement techniques, such as fluoroscopy and the like.

### Delivery Systems

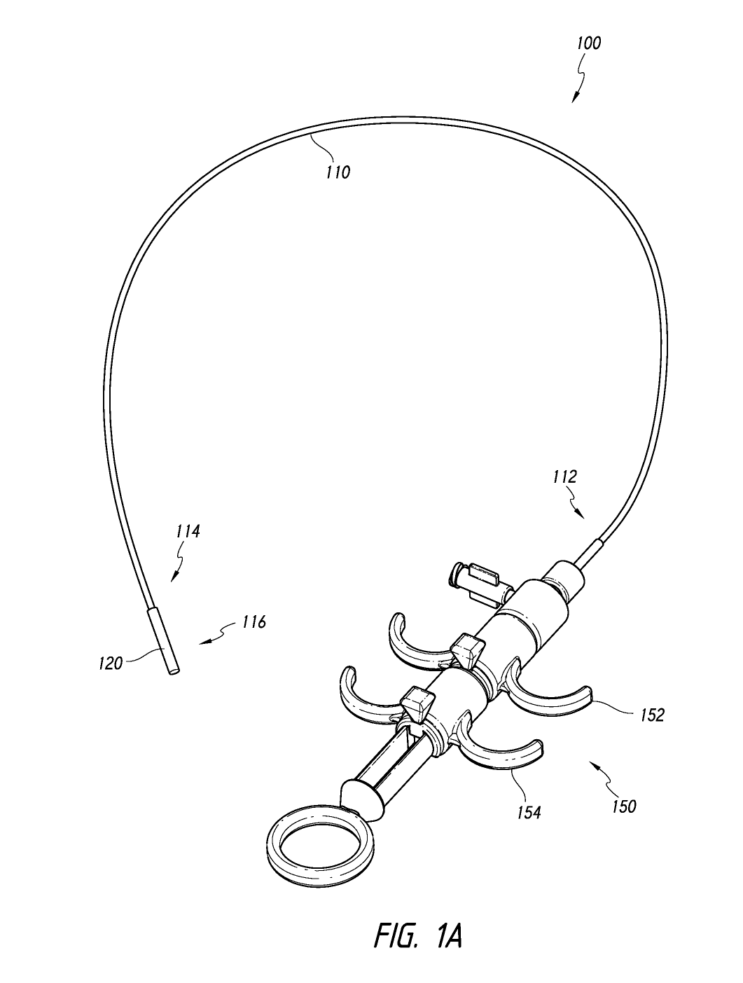

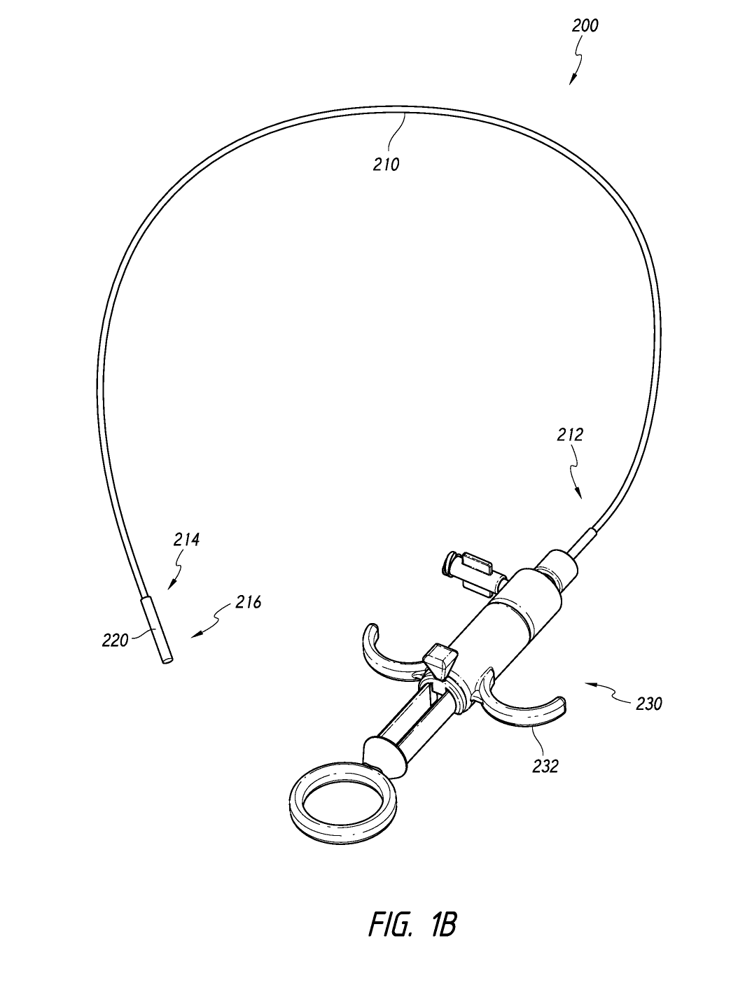

In accordance with some embodiments, a delivery system can control release and expansion of the implant at a target site within the vasculature. For example, FIG. 1A illustrates an embodiment of an implant carrier assembly 100, which can comprise a catheter 110 having a lumen that extends between a proximal portion 112 and a distal portion 114 of the catheter. The catheter 110 can also comprise an engagement section 116, which can be located along a distal portion of the catheter 110, configured to engage and/or restrain an implant positioned therealong. Thus, the implant can be supported, engaged, or restrained along an exterior surface of the catheter.

Similarly, FIG. 1B illustrates an embodiment of an implant carrier assembly 200, which can comprise a catheter 210 having a lumen that extends between a proximal portion 212 and a distal portion 214 of the catheter. The catheter 210 can also comprise an engagement section 216, which can be located along a distal portion of the catheter 210, configured to engage and/or restrain an implant positioned therealong.

In some embodiments, the catheter 110, 210 can define a length from about 50 cm to about 200 cm, from about 70 cm to about 160 cm, or in some embodiments, about 120 cm, with a working length of from about 85 cm to about 140 cm, from about 95 cm to about 130 cm. In accordance with some embodiments, the total length of the implant carrier assembly (with handle) can be about 117 cm, with a working length of 97 cm.

The catheter 110, 210 can be configured to move within a guide sheath when advancing the assembly 100, 200 into a patient for treatment. The proximal portion 112, 212 of the catheter 110, 210 can be configured to be relatively stiff in order to enhance the pushability of the catheter 110, 210 through the guide sheath. Further, the distal portion 114, 214 can be relatively flexible in order to improve the maneuverability and trackability of the catheter 110, 210 as it is advanced through the guide sheath.

The assembly 100, 200 can also comprise an implant or device 120, 220. As shown in FIGS. 1A-1B, the implant 120 can be supported on the engagement section 116, 216 of the catheter 110, 210. The implant or device 120, 220 can be configured as any of the implants or devices disclosed herein or otherwise incorporated by reference.

The delivery system, in accordance with some embodiments, can include a single actuator or multiple actuators that control engagement of the implant with the delivery system. For example, the assembly 100 can also comprise a deployment handle assembly 150 attached to the catheter proximal portion 112. The deployment handle assembly 150 shown in FIG. 1A includes two pull members 152, 154, whereas the deployment handle assembly 230 shown in FIG. 1B includes a single pull member 232. The pull members 152, 154 can be used to release the implant 120 from engagement with the engagement section 116 of the catheter 110, as discussed further herein. Similarly, the pull member 232 can be used to release the implant 120 from engagement with the engagement section 216 of the catheter 210, as also discussed further herein. In some embodiments, both deployment handle assemblies 150, 230 can be used to release distal and proximal portions of the implant 120. Either embodiment can be used with any of the implant embodiments disclosed herein to perform any of the methods and procedures disclosed herein. Other features can include those discussed in co-pending U.S. patent application Ser. No. 14/044,794, filed Oct. 2, 2013, the entirety of the disclosure of which is incorporated herein by reference.

Referring still to FIG. 1A, the deployment handle assembly 150 can comprise separate, dedicated pull members 152, 154 to release each of the distal and proximal portions of the implant 120. The pull members 152, 154 can be pushed and/or pulled to engage with or disengage from the implant 120.

For example, the pull member 152 can be coupled to a first actuation or elongate member, and the pull member 154 can be coupled to a second actuation or elongate member. The first and second elongate members can extend distally toward the engagement section 116. The first and second elongate members can be releasably engageable with respective proximal or distal portion of the implant 120. In use, for example, the pull member 154 can be proximally withdrawn, causing the second elongate member to move proximally and disengage with the proximal or distal end of the implant 120. Further, the pull member 152 can then be proximally withdrawn, causing the first elongate member to move proximally and disengage with the other of the proximal or distal end of the implant 120. The assembly 100 can thereby provide either sequential or simultaneous controlled deployment of the proximal and distal ends of the implant 120. Alternatively, as noted above, the pull members 152, 154 can be pushed to disengage from the implant 120 in a sequential manner.

Additionally, the deployment handle assembly 230 uses a single pull member 232 that can be, for example, moved a first distance to release the distal portion of the implant 220 and moved a second distance to release the proximal portion of the implant 220. The pull member 232 can be coupled to an elongate member that extends distally toward the engagement section 216. The elongate member can be releasably engageable with proximal and distal portion of the implant 220. In use, the pull member 232 can be proximally withdrawn at a first axial distance, causing the elongate member to move proximally and disengage with the proximal or distal end of the implant 220. Further, the pull member 232 can then be proximally withdrawn a second axial distance, which can be greater than the first axial distance, causing the first elongate member to move proximally and disengage with the other of the proximal or distal end of the implant 120. The assembly 100 can thereby provide either sequential or simultaneous controlled deployment of the proximal and distal ends of the implant 120. Alternatively, as noted above, the pull member 232 can be pushed to disengage from the implant 220 in a sequential manner.

In some embodiments, whether a single or multiple pull members are used, the pull members can be pushed distally relative to the handle assembly 150, 230 to cause release of a portion of the implant from engagement with the assembly 100, 200. Thus, the pull members can move in either direction, and in any order, to release one or more portions of the implant (e.g., the distal or proximal ends of the implant), whether sequentially or simultaneously.

The implant carrier assemblies 100, 200 may be used in combination with any of the implants disclosed herein, including variations and combinations thereof.

While various embodiments disclosed herein relate to “non-coil-type” implant support frames, defined as support frames that have do not extend helically in only a single direction (i.e., that reverse direction one or more times), implant support frames that have symmetrical halves that each extend about only a portion of a perimeter of the support frame and are coupled to each other to form a completed support frame, or support frames that do not extend helically at all, some embodiments disclosed herein can comprise “coil-type” implant support frames, defined as support frames that extend helically only in a single direction.

For example, a non-coil-type support frame, such as a symmetrical dual-component implant support frame, can have symmetrical halves formed using dual wires that each extend about only a portion of a perimeter of the support frame and are coupled to each other to form a completed support frame. Such embodiments can provide various advantages over coil-type support frames. Generally, the maximum expanded diameter to which a coil-type support frame can resiliently expand is less than the maximum expanded diameter to which a non-coil-type or symmetrical support frame can resiliently expand. Further, the minimum collapsed diameter or profile of a coil-type support frame is greater than the minimum collapsed diameter or profile of a non-coil-type or symmetrical support frame.

Thus, a non-coil-type or symmetrical support frame can be delivered through a lower profile catheter and/or be released into vessels having a larger diameter than those treatable by a coil-type support frame. Further, even if a coil-type support frame is designed to provide the same minimum collapsed profile as a non-coil-type or symmetrical support frame, the non-coil-type or symmetrical support frame can have a greater maximum expanded diameter than the coil-type support frame. Furthermore, even if a coil-type support frame is designed to provide the same maximum expanded diameter as a non-coil-type or symmetrical support frame, the non-coil-type or symmetrical support frame can have a smaller minimum collapsed profile than a coil-type support frame.

In addition, because a non-coil-type or symmetrical support frame need not be torsionally constrained when mounted or supported on a support member (e.g., a catheter), the non-coil-type or symmetrical support frame will not tend to exert any torsional force or torque on the support member. Thus, the support member carrying a non-coil-type or symmetrical support frame need not provide any significant torque resistance to accommodate torque exerted by the non-coil-type or symmetrical support frame (which would otherwise be necessary if a coil-type support frame were used).

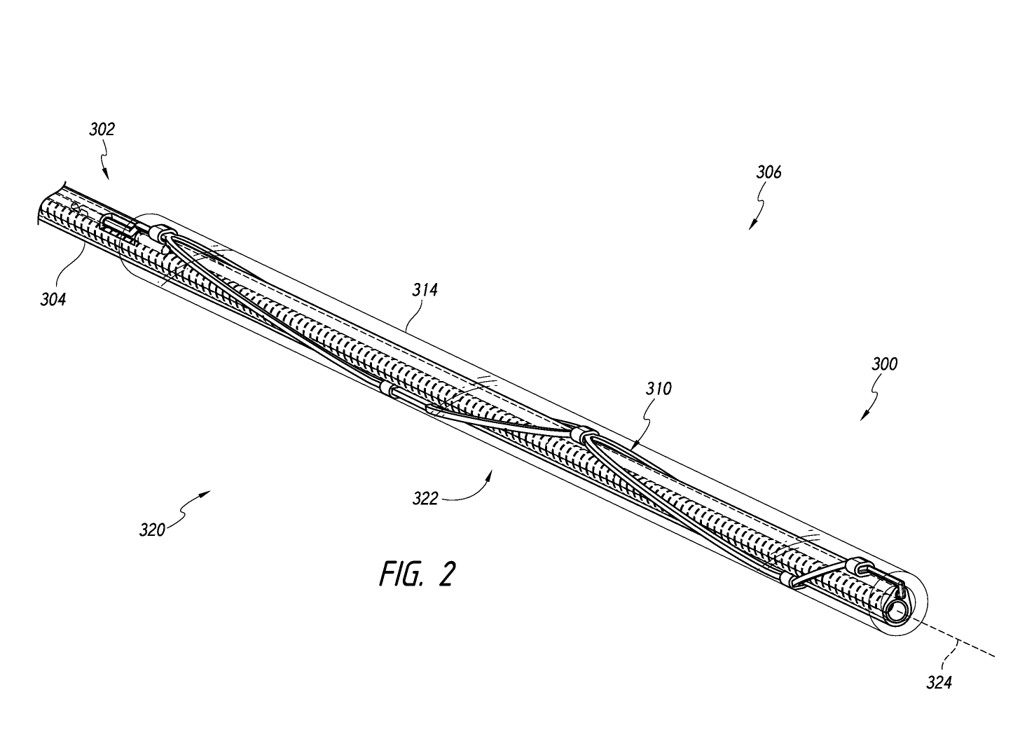

Some embodiments of a non-coil-type or symmetrical support frame are illustrated in FIGS. 2, 6A, 6B, 9-13C, 14, 16-31, 33A, 33B, and 35A-35E. FIG. 2 shows a side view of an implant 300 supported on an implant carrier assembly 302, which can comprise a catheter 304, to form an implant delivery system 306, according to some embodiments. The implant 300 can comprise a support frame 310 having one or more rings or support elements 312 and a membrane or occlusive member 314 that encloses and/or extends within a lumen of the support frame 310. The carrier assembly 302 can comprise a distal portion 320 having an implant support section 322.

As with any of the embodiments disclosed herein, the carrier assembly 302 can optionally comprise one or more lumens that enable the assembly 302 to be delivered over the wire (“OTW”) to a target or treatment site. However, configurations and delivery method can also be implemented that omit the OTW capability and instead permit delivery through a catheter to the target site. FIG. 2 illustrates a guidewire 324 extending through a lumen of the carrier assembly 302. As discussed further below, the implant 300 can be releasably coupled to the carrier assembly 302. In some embodiments, proximal and/or distal sections of the implant 300 can be engaged with corresponding structures of the carrier assembly 302. These and other structures are disclosed herein.

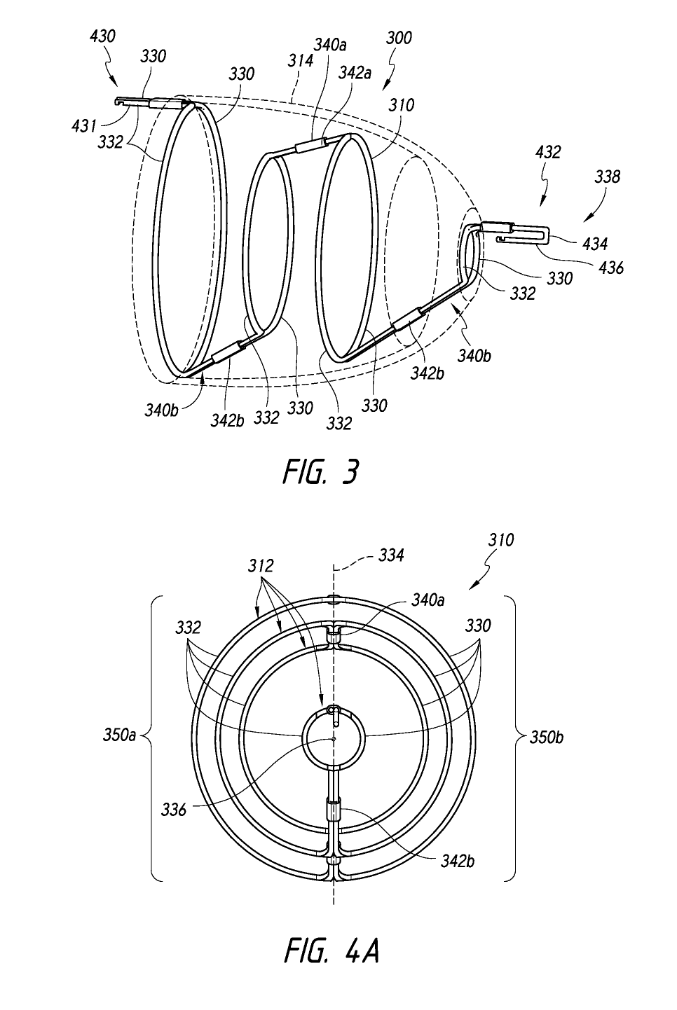

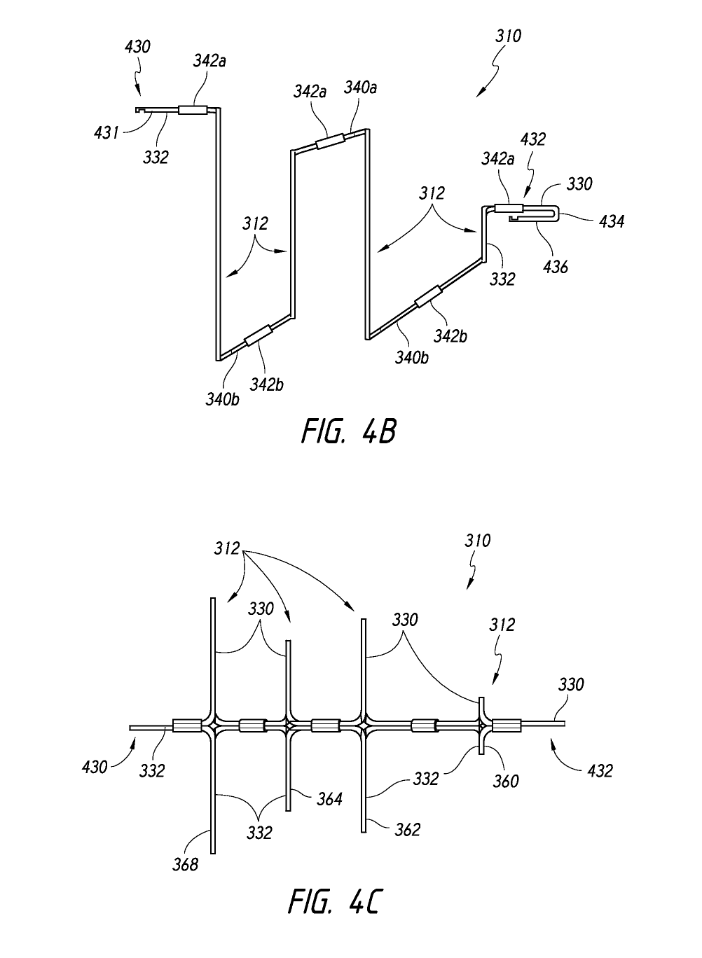

FIG. 3 illustrates a perspective view of the implant support frame 310 and the membrane 314 (shown in dashed lines) in an expanded state, according to some embodiments. In some embodiments, the support elements 312 of the support frame 310 can comprise separate wires 330, 332 that are generally mirror images of each other along a longitudinal center plane 334 (extending through the central axis 336) of the frame 310, as illustrated in FIG. 4A. The wires 330, 332 can extend from a first or proximal end portion 337 to a second or distal end portion 338 of the frame 310. Additional wires (e.g., 3, 4, or more wires) may be coupled together as disclosed herein. In some embodiments, whether a single or multiple wires are used, the wires can comprise a nitinol or titanium material.

The wires 330, 332 may be coupled together and extend axially along axial portions 340a and 340b. The axial portions 340a may be radially opposite the axial portions 340b across the central axis 336. Along the axial portions 340a, 340b, the wires 330, 332 may be adjacent and/or contacting. The wires 330, 332 may be joined or coupled together with connectors 342a at the axial portions 340a and with connectors 342b at the axial portions 340b. As shown in FIGS. 3-4C, the connectors 342a, 342b may be bands, cuffs, rings, clips, coil windings, combinations thereof, and the like. The connectors 342a, 342b may also be adhesive, glue, welding, combinations thereof, and the like. The connectors 342a, 342b may also be radiopaque for visualization.

The wires 330, 332 may extend circumferentially along circumferential portions 350a and 350b. The circumferential portions 350a may be radially opposite the circumferential portions 350b across or about the central axis 336. Each of the circumferential portions 350a, 350b may extend from an axial portion 340a to an axial portion 340b, radially opposite the axial portion 340a. The circumferential portions 350a, 350b can collectively define the one or more rings or support elements 312, as illustrated in FIG. 4A. The axial portions 340a, 340b between which a single circumferential portion 350a or 350b extends may be axially displaced relative to each other. Each wire 330, 332 may extend entirely on a respective radial side of the frame 310. Each circumferential portion 350a or 350b may extend along at least a portion of a cylindrical path in a clockwise circumferential direction toward a given axial portion 340a or 340b, and each circumferential portion 350a or 350b may extend along at least a portion of the cylindrical path in a counterclockwise circumferential direction away from the given axial portion 340a or 340b. Each wire 330, 332 may contact all or less than all of the connectors 342a, 342b.

Thus, the interconnections of the separate wires 330, 332 can lie substantially in a common plane. However, the separate wires 330, 332 can also form interconnections that are not mirror images or that do not lie in a common plane. For example, in some embodiments in which the frame 310 defines a generally tubular shape, the interconnections can be located at different and varied circumferential locations. For example, the interconnections can be distributed across one, two, three, four, five, or more circumferential locations. The pattern can be a repeating pattern or randomized, which can provide a desired flexibility or strength characteristics for the frame.

Additionally, the separate wires 330, 332 can be of a common gauge or can have different gauges, in order to impart a desired strength characteristics.

The support elements 312 of the support frame 310 can be generally circular and may be formed as loops or hoops. However, the support elements 312 can be formed in any of a variety of shapes, including square, triangle, rectangle, oval, or other polygons (having five, six, seven, eight, nine, or more sides). Additionally, as illustrated in FIGS. 3-4C, at least one of the support elements 312 can have an expanded or outer diameter different than one or more of the other support elements 312.

For example, the support elements 312 can have expanded diameters from about 2 mm to about 30 mm or more. In some embodiments, the expanded diameter of a given support element 312 can be between about 2 mm and about 20 mm, between about 3 mm and about 16 mm, between about 4 mm and about 12 mm, or between about 5 mm and about 8 mm. For example, a given support element 312 can have an expanded diameter of about 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, or more.

In some embodiments, each of the support elements 312 can have an expanded diameter that is different from the other support elements 312 of the frame 310.

For example, as shown in FIG. 4C, a first support element 360 can have an expanded diameter of about 2 mm, a second support element 362 can have an expanded diameter of about 8 mm, a third support element 364 can have an expanded diameter of about 6 mm, and a fourth support element 368 can have an expanded diameter of about 10 mm.

According to some embodiments, the variation of expanded diameter sizes of the support elements 312 can allow the implant 300 to self-adjust to different vessel diameters and/or provide anti-migration benefits. In accordance with some embodiments, the support frame 310 illustrated in FIG. 4C can be placed into a vessel having a diameter of between less than 2 mm and about 8 mm.

For example, when the embodiment of FIG. 4C is placed in a smaller size vessel, the larger support elements 362, 368 can extend longitudinally or axially within the vessel in an oval shape that tracks the inner wall of the vessel (extending in a slanted direction relative to a longitudinal axis of the vessel), while the smaller support elements 360, 364 may be fully expanded into apposition with the vessel wall and oriented generally perpendicularly relative to the longitudinal axis of the vessel. Further, when the embodiment of FIG. 4C is placed in a larger size vessel, the larger support elements 362, 368 will tend to extend more perpendicularly relative to the longitudinal axis of the vessel (more so than in a small vessel) while being fully expanded into apposition with the vessel wall, while the smaller support elements 360, 364 may not tend to provide full contact or engagement with the vessel wall if the expanded diameters of the support elements 360, 364 is less than the internal diameter of the vessel.

Such embodiments can advantageously permit the implant to be used with a range of vessel sizes.

Further, as similarly noted above, the implant 300 can advantageously be used in a greater range of vessel diameters than coil-type implant support frames that extend helically in a single direction. For example, some embodiments, such as that illustrated in FIGS. 3-4C, can be used in vessel diameters between 2 mm and greater than 20 mm. Such a broad range of vessel diameters for a single implant has not been possible using coil-type implant support frames that extend helically in a single direction, due to the limitations of the coil-type support frames.

For example, with some non-coil-type embodiments, such as the implant 300, the ratio of the minimum collapsed profile to the maximum expanded profile (which can be measured in a diameter that circumscribes the collapsed or expanded profile) can be between about 1:20, about 1:18, about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

Additionally, due to the exceptional minimum collapsed profile achievable using some non-coil-type embodiments, such as the implant 300, the implant 300 can be fitted into a delivery catheter having a profile of less than about 5 Fr, less than about 4 Fr, less than about 3 Fr, or smaller.

In accordance with some embodiments, the support frame 310 can also be formed using a plurality of support elements 312 that are interconnected with one or more backbone members or axial portions. The backbone members can extend in a longitudinal or axial direction such that the support elements 312 are spaced apart along the longitudinal axis of the support frame 310. Thus, instead of having a pair of wires 330, 332, the support frame 310 can be formed using a series of same-sized or differently sized rings or support elements 312 that are interconnected to each other using backbone members that are welded or otherwise coupled to the adjacent rings to form a cylindrical structure that has a constant diameter (in the case of using support elements that have a common diameter) or a cylindrical structure that has a varying diameter (in the case of using support elements that have different diameters, as in FIG. 3).

The backbone member(s) can extend intermittently on opposing sides of the support elements 312 as shown in FIG. 3) or continuously along a single side of the support elements 312. When the backbone members or axial portions extend intermittently along opposing sides of the support elements 312, as in the case of axial portions 340a, 340b, the length of the support frame 310 can be longitudinally stretched when positioned onto the carrier assembly 302. Accordingly, when expanding from the collapsed configuration, the support elements 312 can be longitudinally drawn towards each other and the outer profile or diameter of the implant 300 can increase. Further, when only a single backbone member or axial portion is used, the support elements 312 can be coupled to the backbone member at positions spaced apart from each other their along. In such embodiments, the support frame 310 may not tend to longitudinally elongate when in the collapsed configuration. However, in either implementation of the backbone member(s), the support elements 312 can be biased towards a position that is substantially orthogonal relative to the backbone member(s). In being “substantially orthogonal,” the support element can assume a position that is oriented within about 35° relative to a normal line of a longitudinal axis of a backbone member.

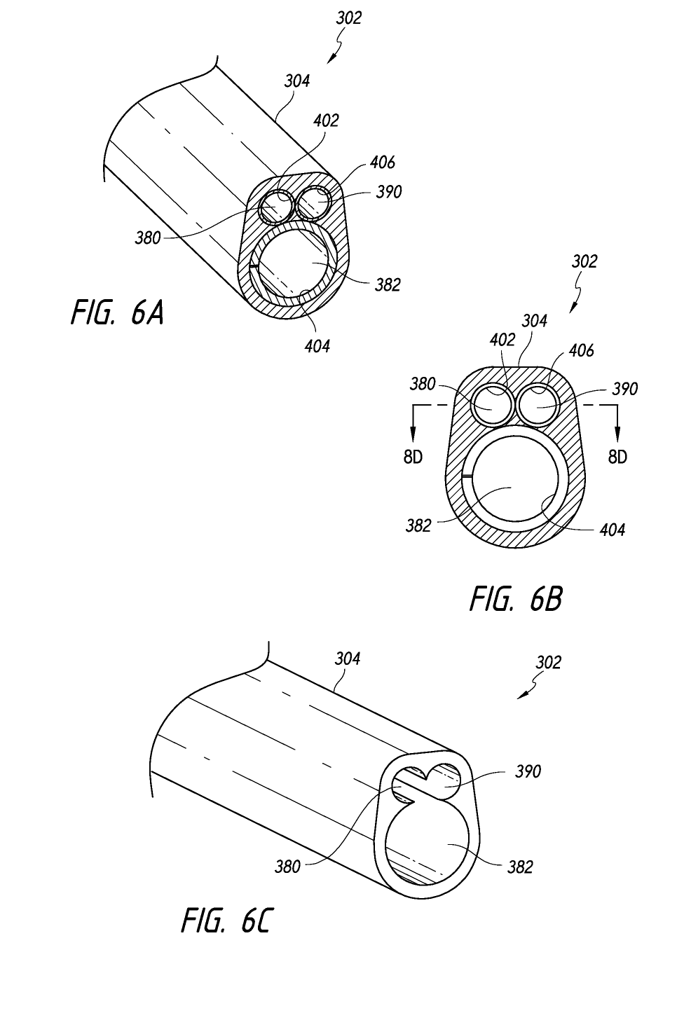

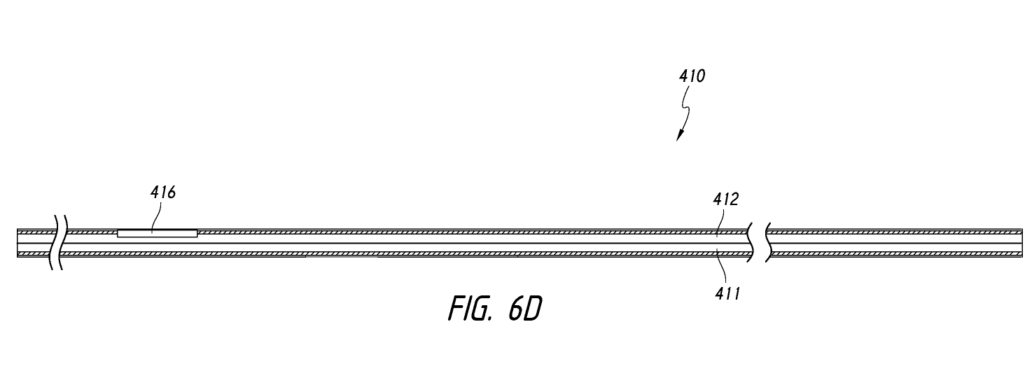

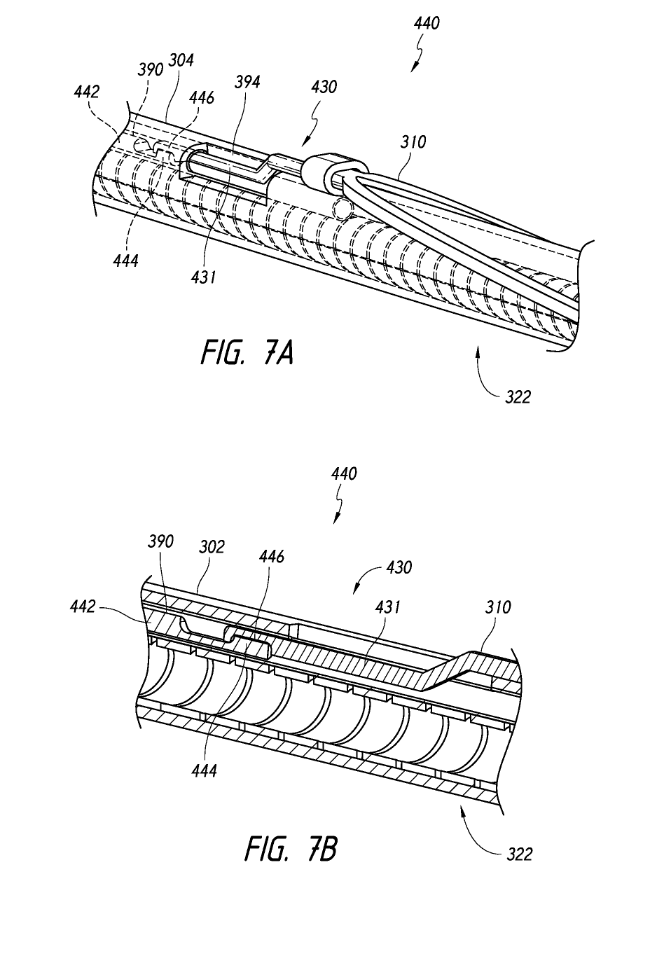

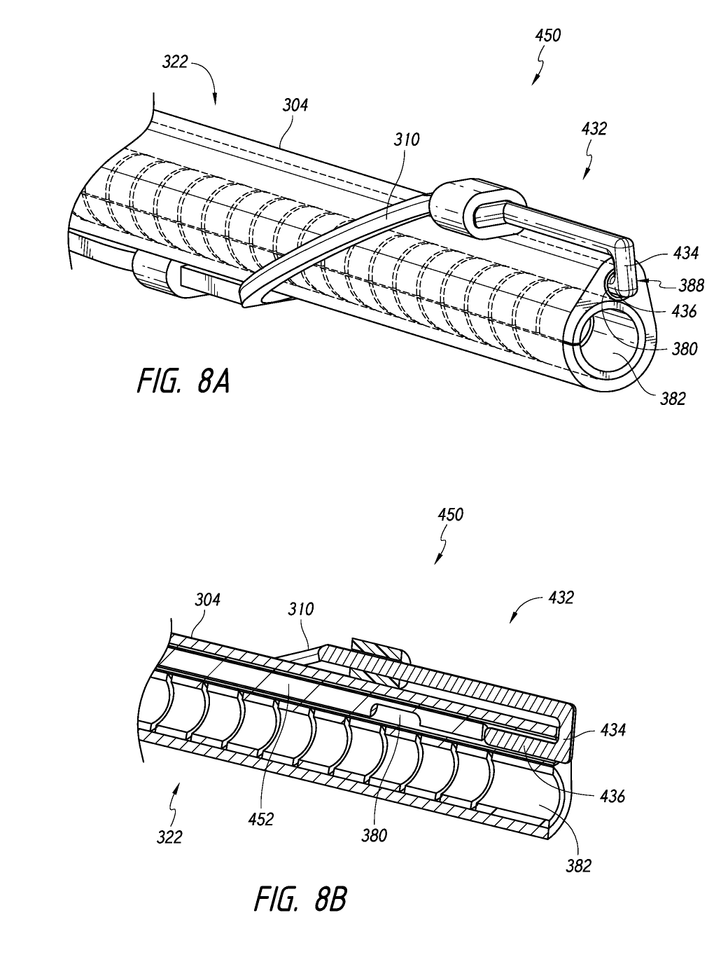

As also illustrated in FIGS. 3 and 4B, the support frame 310 can comprise proximal and distal coupling members 430, 432. The coupling members 430, 432 can comprise portions of the wires that form the support elements 312 of the implant support frame 310, thus comprising the same material or being integrally formed with the support elements 312. The coupling members 430, 432 can each comprise one or both of the wires 330, 332. For example, as shown in FIGS. 3-4C, the proximal coupling member 430 can comprise the wire 332, but not the wire 330. Further, FIGS. 3-4C also show that the distal coupling member 432 can comprise the wire 330, but not the wire 332. However, the coupling members 430, 432 can also be separate components that are joined to the support elements 312. The coupling members 430, 432 can extend away from the support elements 312 of the implant support frame 310. The coupling members 430, 432 can each be configured to engage with a respective engagement mechanism of the carrier assembly 302, as discussed below with respect to FIGS. 6A-7B.

FIGS. 3 and 4B illustrate that in accordance with some embodiments, the proximal coupling member 430 can comprise a proximalmost portion 431 that can comprise a substantially linear or straight configuration. As such, as discussed herein, the proximalmost portion 431 can be inserted into a catheter in a proximal direction while only causing slight deflection of the proximal coupling member 430. However, the distal coupling member 432 can be configured to comprise a bend 434 such that a distalmost portion 436 extends proximally from the bend 434. As such, as discussed herein, the distal coupling member 432 can be inserted into a catheter in a proximal direction as well.

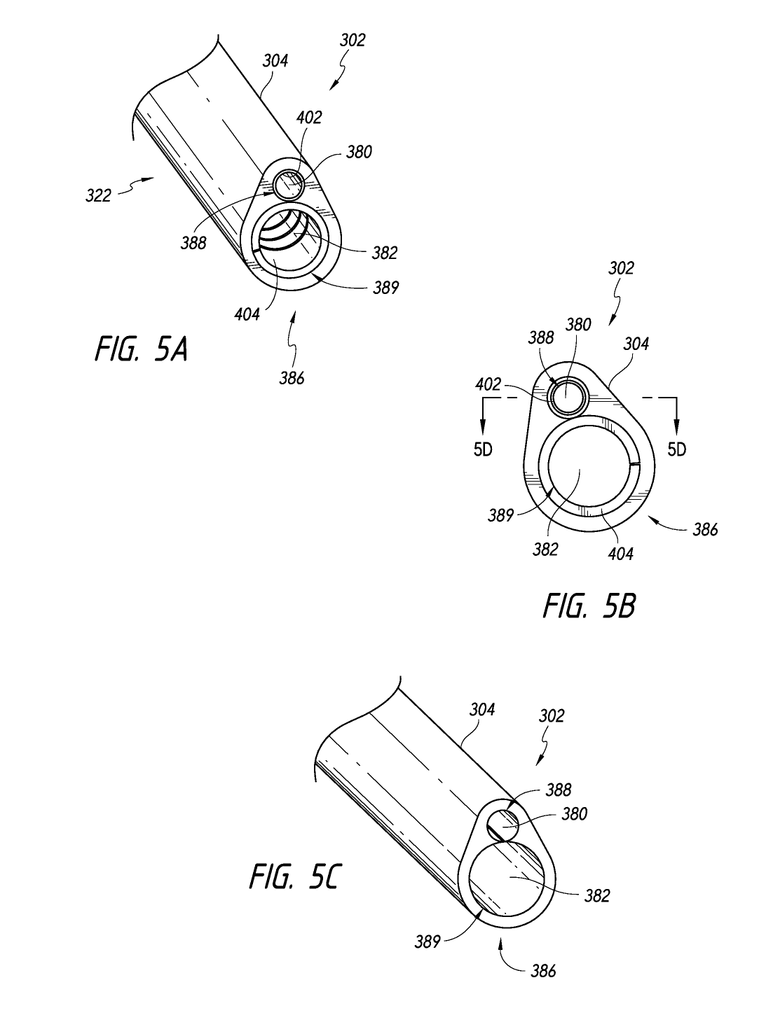

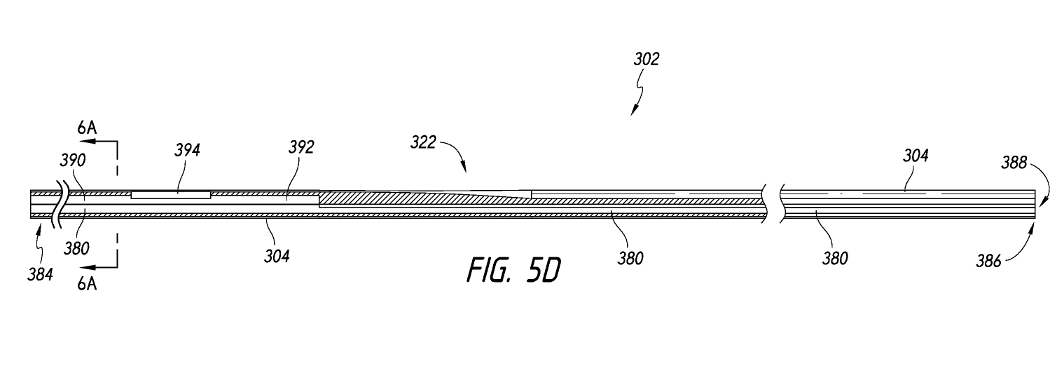

FIGS. 5A-5D illustrate different views and aspects of an embodiment of the catheter 304 of the carrier assembly 302. FIGS. 5A-5C illustrate views of the implant support section 322 of the catheter 304. FIG. 5D illustrates the implant support section 322 as well as other proximal sections of the catheter 304.

As illustrated in FIGS. 5A and 5B, in the implant support section 322, the catheter 304 can comprise at least one lumen extending therethrough. In some embodiments, at least one lumen can be configured to receive a respective elongate actuation member, which can be in the form of an elongate wire, and/or a portion of an implant device to releasably secure a portion of an implant device relative to the catheter 304. FIGS. 5A-5C illustrate an enlarged view of the catheter 304 along the implant support section 322, along which the catheter 304 comprises an actuator lumen 380 and a guidewire lumen 382. The actuator lumen 380 and the guidewire lumen 382 can extend from a proximal portion 384 of the catheter 304 to a distal portion 386 of the catheter 304. The actuator lumen 380 can terminate at a distal aperture 388, and the guidewire lumen 382 can terminate at a distal aperture 389. The distal aperture 388 can be configured to receive a portion of an implant device, according to some embodiments (see FIGS. 8A and 8B).

FIG. 5D illustrates a top, cross-sectional view of the catheter 304, illustrating the actuator lumen 380 extending from the proximal portion 384 to the distal portion 386. In addition, the catheter 304 can also comprise a second actuator lumen 390. The actuator lumen 380 can be a first actuator lumen that extends along an entire length of the catheter 304 while the second actuator lumen 390 can extend along almost the entire length of the catheter 304 except along the implant support section 322. Thus, the second actuator lumen 390 can terminate or end at a distal end 392 at or adjacent to a proximal end portion of the implant support section 322.

FIGS. 6A-6C illustrate a cross-sectional view of the catheter 304 taken along section lines 6AC-6AC of FIG. 5D. FIG. 6A-6C illustrate the positioning of the first and second actuator lumens 380, 390 and the guidewire lumen 382 relative to each other, according to some embodiments.

As discussed herein, both the first and second actuator lumens 380, 390 can be configured to receive respective elongate actuation members, which can be in the form of elongate wires. In some embodiments, the first and second actuator lumens 380, 390 can each comprise an inner diameter of between about 0.004 inches and about 0.008 inches, and in some embodiments, between about 0.005 inches and about 0.007 inches, or in some embodiments, about 0.005 inches or about 0.006 inches. Further, the guidewire lumen 382 can be configured to receive a guidewire therethrough. The guidewire lumen 382 can comprise an inner diameter of between about 0.020 inches and about 0.030 inches, or in some embodiments, between about 0.022 inches and about 0.028 inches, between about 0.024 inches and about 0.026 inches, or in some embodiments, about 0.025 inches. Thus, accounting for some radial thickness in the catheter material, the cross section of the catheter 304 (whether that of FIG. 5B or 6B), can be circumscribed by a diameter of between about 0.030 inches and about 0.040 inches, or in some embodiments, between about 0.033 inches and about 0.039 inches, between about 0.035 inches and about 0.037 inches, or in some embodiments, about 0.034 inches or about 0.036 inches. In accordance with some embodiments, the elongate actuation members and/or the guidewire used in the system can each comprise a diameter of between about 0.004 inches and about 0.006 inches, or in some embodiments, about 0.005 inches. Due to such advantageous features, some embodiments of the system 306 can fit into a 4 Fr guide catheter.

As shown in FIG. 5D, the catheter 304 can comprise a cutout or aperture 394 adjacent to the distal end 392 of the second actuator lumen 390. As discussed and shown further herein, the aperture 394 can be configured to receive a portion of an implant device in order to permit the implant device to be engaged within the second actuator lumen 390 by a respective elongate actuation member. In some embodiments, the aperture 394 can be a distal aperture of the second actuator lumen 390, such that the aperture 394 has a cross-sectional profile that is equivalent to the inner diameter or inner profile of the second actuator lumen 390 (e.g., a circular profile). In such embodiments, a portion of the implant device can be inserted longitudinally into the aperture 394. However, the aperture 394 can also be a window into the second actuator lumen 390 that extends along a longitudinal length of the second actuator lumen, sufficient to permit a portion of an implant device to be inserted therethrough (e.g., radially and/or longitudinally into the second actuator lumen 390).

Additionally, in accordance with some embodiments, as illustrated in FIGS. 5A-5C and 6A-6C, the first and/or second actuator lumens 380, 390 and/or the guidewire lumen 382 can comprise an insert or liner component that can be inserted into the respective lumen to provide desired frictional or structural characteristics or properties. For example, the first actuator lumen 380 can comprise an insert component 402, the guidewire lumen 382 can comprise an insert component 404, and/or the second actuator lumen can comprise an insert component 406. FIGS. 5A, 5B, 6A, and 6B illustrate the presence of the insert components 402, 404, 406. FIGS. 5C and 6C illustrate the catheter 304 with the insert components 402, 404, 406 removed from the respective first and second actuator lumens 380, 390 and the guidewire lumen 382. Although the illustrated embodiment may include insert components, the catheter 304 can be configured to exclude any additional components, such as the insert components.

Referring now to FIG. 6D, an alternative embodiment of a catheter 410 is shown in which first and second lumens 412, 414 extend along the entire length of the catheter 410. The catheter 410 can be used in the same manner as the catheter 304. However, the catheter 410 can also enable the system to provide dual distal apertures (at the distal ends of the first and second lumens 412, 414), which can each be used to support and/or engage a distal portion of an implant device. Additionally, the catheter 410 can comprise a cutout or aperture 416, which can be used in the same manner as the aperture 394 discussed above.

FIGS. 7A and 7B show perspective views of a proximal engagement mechanism 440 for engaging and supporting the implant support frame 310 on the carrier assembly 302, according to some embodiments. The proximal engagement mechanism 440 can comprise the proximal coupling member 430 of the implant support frame 310. Further, the proximal engagement mechanism 440 can also comprise a portion or section of the catheter 304. Furthermore, the proximal engagement mechanism 440 can also comprise a portion or section of an elongate actuation member 442 of the carrier assembly 302 that is disposed within the second actuator lumen 390 of the catheter 304.

As shown in FIGS. 7A and 7B, the elongate actuation member 442 can comprise a recess and/or protrusion 444 at its distal end that is configured to engage with the proximalmost portion 431 of the proximal coupling member 430 of the support frame 310. Further, the proximalmost portion 431 can comprise a corresponding recess and/or protrusion 446 configured to engage with the recess and/or protrusion 444 of the elongate actuation member 442. Accordingly, when longitudinally overlapping each other and disposed within the second actuator lumen 390, the actuation member 442 and the proximalmost portion 431 can radially overlap each other (e.g., overlap each other at least partially along an axial length or in a longitudinal direction) such that they are maintained in a substantially fixed longitudinal engagement relative to each other within the second actuator lumen 390. A wall of the second actuator lumen 390 can radially constrain (e.g., constrain movement in a direction transverse or perpendicular to the longitudinal axis) the proximal coupling member 430 against the protrusion 444, thereby preventing or restricting disengagement between the proximal coupling member 430 and the protrusion 444. The same principle can be effective in maintaining and removing an engagement between the distal coupling member 432 and a protrusion, as discussed further herein. Thus, in some embodiments, the proximal and distal engagement mechanisms 440, 450 can permit the proximal and distal coupling members 430, 432 to be radially and longitudinally constrained such that the support frame 310 is maintained in an engaged state relative to the first and second actuation members 442, 452.

Accordingly, in some embodiments, movement of the actuation member 442 can be transmitted directly to the proximalmost portion 431. This mechanical engagement between the actuation member 442 and the proximalmost portion 431 can allow the user to control the position of the proximalmost portion 431 and to maintain engagement of the proximalmost portion 431 within the second actuator lumen 390 or distally advance the actuation member 442 until the proximalmost portion 431 is able to fully exit the aperture 394, thereby releasing the proximalmost portion 431 from engagement with the actuation member 442. In accordance with some embodiments, engagement between the proximalmost portion 431 and the actuation member 442 can be maintained until the corresponding recesses and/or protrusions 444, 446 appear through or exit the window or aperture 394. Thereafter, the proximal portion of the support frame 310 can freely expand.

Further, in accordance with some embodiments, FIGS. 8A and 8B show side views of a distal engagement mechanism 450 for engaging and supporting the implant support frame 310 on the carrier assembly 302. As illustrated, the distal engagement mechanism 450 can comprise the distal coupling member 432 of the implant support frame 310. Further, the distal engagement mechanism 450 can also comprise a portion or section of the catheter 304. Furthermore, the distal engagement mechanism 450 can also comprise a portion or section of a second elongate actuation member 452 of the carrier assembly 302 that is disposed within the first actuator lumen 380 of the catheter 304.

As shown FIGS. 8A and 8B, the distal coupling member 432 can engage with the catheter 304. For example, the distalmost portion 436 can extend proximally into the aperture 388 of the first actuator lumen 380. As shown in FIG. 8B, the distalmost portion 436 can longitudinally abut a distal end of the second actuation member 452. Thus, when the user desires to disengage the distalmost portion 436 from the first actuator lumen 380, the user can exert a pushing force in the distal direction on the second actuation member 452 such that the second actuator member 452 pushes the distalmost portion 436 out of the first actuator lumen 380. In some embodiments, this type of disengagement can be performed in delivery systems that incorporate and engagement mechanism, such as the proximal engagement mechanism 440 discussed above, by which the distalmost portion 436 can be maintained within the first actuator lumen 380 by virtue of a proximally oriented tension or force exerted on the proximal coupling member 430 by the proximal engagement mechanism 440. In such embodiments, the implant device can be released by initially releasing the distalmost portion 436, as discussed herein.

However, as illustrated in discussed above with respect to the proximal engagement mechanism 440, the distal engagement mechanism 450 can also be configured such that the second actuation member 452 comprises a protrusion that is configured to engage with the distalmost portion 436 such that the second actuation member 452 can be longitudinally moved in order to pull or push the distalmost portion 436, similar to the interaction between the (first) actuation member 442 and the proximalmost portion 431. Thus, in such embodiments, this type of engagement can allow the distalmost portion 436 to be constrained within the first actuator lumen 380 regardless of the engagement or disengagement of the proximal coupling member 430 of the support frame 310. Thus, in such embodiments, the implant device can be released by initially releasing the proximalmost portion 431 or by initially releasing the distalmost portion 436.

Therefore, in accordance with some embodiments, at least one or both of the proximal and distal coupling members 430, 432 can comprise engagement recesses and/or protrusions, such as a notch, into which a corresponding recess and/or protrusion of a respective one of the first or second actuation members 442, 452 can be fitted in order to restrict longitudinal or axial movement therebetween. When at least one or both of the proximal and distal coupling members 430, 432 is so engaged, the support frame can be drawn or stretched along the implant support section 322 of the carrier assembly 302. Additionally, in some embodiments, the catheter 304 can comprise a single actuator lumen that is used to engage only a proximal portion of the implant 300, and a distal portion of the implant 300 can be secured relative to a distal section of the catheter 304 until the proximal portion of the implant 300 is distally advanced, thereby allowing the implant to begin to expand and become disengaged from the distal section of the catheter.

In some embodiments, the recesses and/or protrusions of the proximal and distal engagement mechanisms 440, 450 can be formed on the carrier assembly 302. For example, the recesses and/or protrusions can be formed as radial notches in the carrier assembly 302 or as depressions in an outer surface of the carrier assembly 302. The proximal and distal engagement mechanisms 440, 450 also be attached or coupled to the carrier assembly 302, such as by welding or adhesive means. In some embodiments, as shown in FIGS. 7B and 8B, the proximal and distal engagement mechanisms 440, 450 can be formed as notches in elongate wires 442, 452, which create the protrusions 444, 446.

Although in some embodiments, the proximal and distal engagement mechanisms 440, 450 can remain at a fixed longitudinal position relative to the carrier assembly 302, the proximal and distal engagement mechanisms 440, 450 can also be moved relative to the carrier assembly 302 in order to facilitate engagement or disengagement of the support frame 310 to or from the proximal and distal engagement mechanisms 440, 450.

The proximal and distal coupling members 430, 432 can be releasably engaged by the proximal and distal engagement mechanisms 440, 450. In some embodiments, the engagement can be substantially only a mechanical engagement, while in other embodiments, release of the engagement can be actuated by overcoming an adhesive. Further, the proximal and distal coupling members 430, 432 can be continuous with the protrusions 444, 446, such that an electrolytic detachment mechanism can be used to break the connection between the carrier assembly 302 and the support frame 310.

Additionally, in embodiments using a mechanical locking mechanism, in order to assemble the support frame 310 with the carrier assembly 302, the carrier assembly 302 can be positioned within a guide catheter (not shown) such that the support section 322 of the carrier assembly 302 is positioned distally beyond a distal end of the guide catheter 304. In this position, the proximal engagement mechanism 440 (which can comprise a recess, protrusion, or aperture on an outer surface of the catheter 304) can be engaged with the proximal coupling member 430, and the carrier assembly 302 can then be drawn proximally into a lumen of the guide catheter while maintaining engagement between the proximal engagement mechanism 440 and the proximal coupling member 430. Once received within the lumen of the guide catheter, the proximal engagement mechanism 440 is radially constrained in engagement with the proximal coupling member 430, thereby longitudinally constraining movement of the proximal coupling member 430 relative to the proximal engagement mechanism 440. When completed, the support member assembly can be further proximally withdrawn into and relative to the guide catheter until the distal engagement mechanism 450 and the distal coupling member 432 are positioned adjacent to a distal end of the catheter 304. At that point, the distal engagement mechanism 450 and the distal coupling member 432 can be aligned or engaged with each other and the carrier assembly 302 can be further proximally withdrawn into and relative to the catheter 304 until the distal engagement mechanism 450 is fully received within the lumen 462 of the catheter 304. Thereafter, the assembly can be used, with the implant device being releasable from the carrier assembly 302 by distally advancing the proximal engagement mechanism 440 until the proximal coupling member 430 is exposed from within the guide catheter lumen, thereby releasing the support frame 310.

Furthermore, the membrane 314 can be positioned on top of the carrier assembly 302 prior to placing the support frame 310 onto the carrier assembly 302 or drawn or pulled under the support frame 310 all the support frame is initially engaged or fitted over the carrier assembly 302, in a manner similar to that disclosed in co-pending U.S. patent application Ser. No. 14/044,794, filed on Oct. 2, 2013, the entirety of the disclosure of which is incorporated herein by reference.

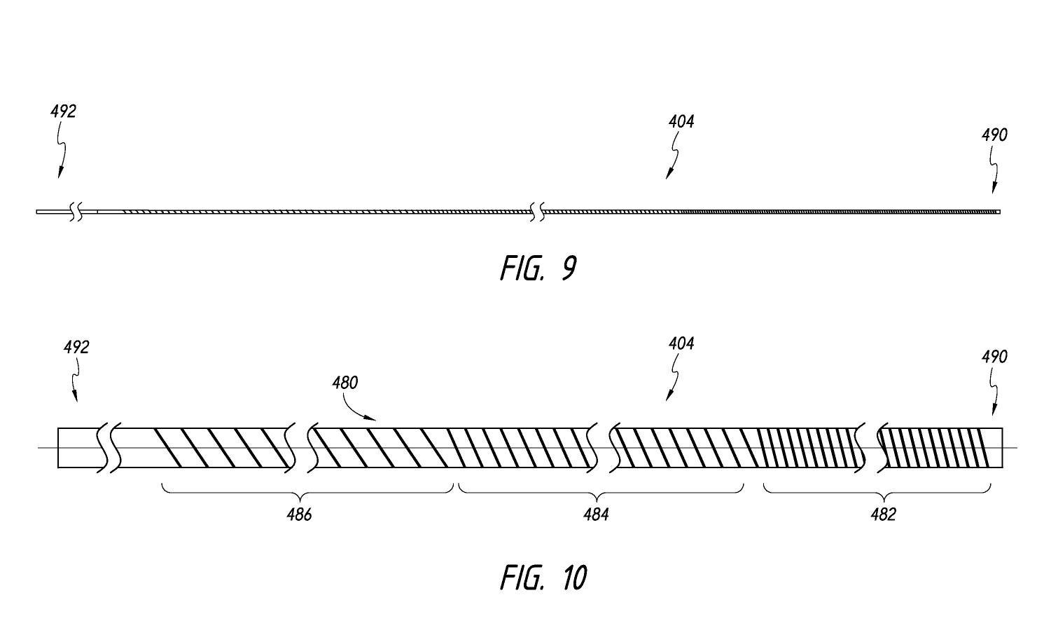

Referring now to FIGS. 9 and 10, as noted above, the guidewire lumen 382 can comprise an insert component 404. FIGS. 9 and 10 illustrate an embodiment of the insert component 404. The insert component 404 can be configured as a hypotube having a helical cut or series of cuts that form a cut pattern 480. The cut pattern 480 can be configured to provide a plurality of zones that each exhibit bending properties that can be different from bending properties of an adjacent zone.

For example, as illustrated FIG. 10, the cut pattern 480 can comprise first, second, and third zones 482, 484, 486 extending along the length of the insert component 404. The first zone 482 can comprise a helical cut having a pitch that is different from a pitch of a helical cut in either the second or third zones 484, 486. Thus, the first zone 482 can have a different bending strength compared to the second or third zones 484, 486. The second and third zones 484, 496 can similarly exhibit bending strength that are different from each other and from the first zone 482.

However, one or more or all of the zones can exhibit same bending strength a single zone of the cut pattern 480. In the embodiment illustrated in FIG. 10, the pitch of the helical cut of each zone increases toward a distal end 490 of the insert component 404. Further, a proximal end 492 of the insert component 404 can be devoid of any cut, such that the insert component 404 exhibits a maximum bending strength along its proximal portion.

In some embodiments, the cut pattern 480 can extend along only that portion of the insert component 404 that extends along the implant support section 322. In such embodiments, the remainder of the insert component 404 can also be devoid of any cut, such that the insert component 404 exhibits a maximum bending strength therealong while providing a different, lower, and possibly variable bending strength along the length of the implant support section 322.

However, the cut pattern 480 can also extend along the entire length of the catheter and implant delivery assembly. The cut pattern 480 can provide various zone of flexibility that can each have the same or different lengths. For example, the cut pattern 480 can begin just proximal of the distal end 490 and have a pitch in the first zone 482 of between about 70° and about 80° for between about 1 inch to about 4 inches, then, moving proximally, change the pitch in the second zone 484 to between about 60° and about 70° for between about 4 inches to about 14 inches, and then, moving proximally, change the pitch in the third zone 486 to between about 50° and about 60° for between about 15 to about 30 inches. In some embodiments, the first zone 482 can comprise a pitch of about 76° and a length of about 2 inches, the second zone 484 can comprise a pitch of about 66° and a length of about 10 inches, and the third zone 486 can comprise a pitch of about 56° and a length of about 24 inches. The changes in pitch can be abrupt or gradual. The total length of the helical cut pattern can be between about 30 inches and about 40 inches, and in some embodiments, about 36 inches.

Further, the insert component 404 can comprise an outer diameter of between about 0.020 inches and about 0.025 inches, or in some embodiments, about 0.0205 inches. The inner diameter of the insert component formed four can be between about 0.014 inches and about 0.018 inches, or in some embodiments, about 0.0165 inches.

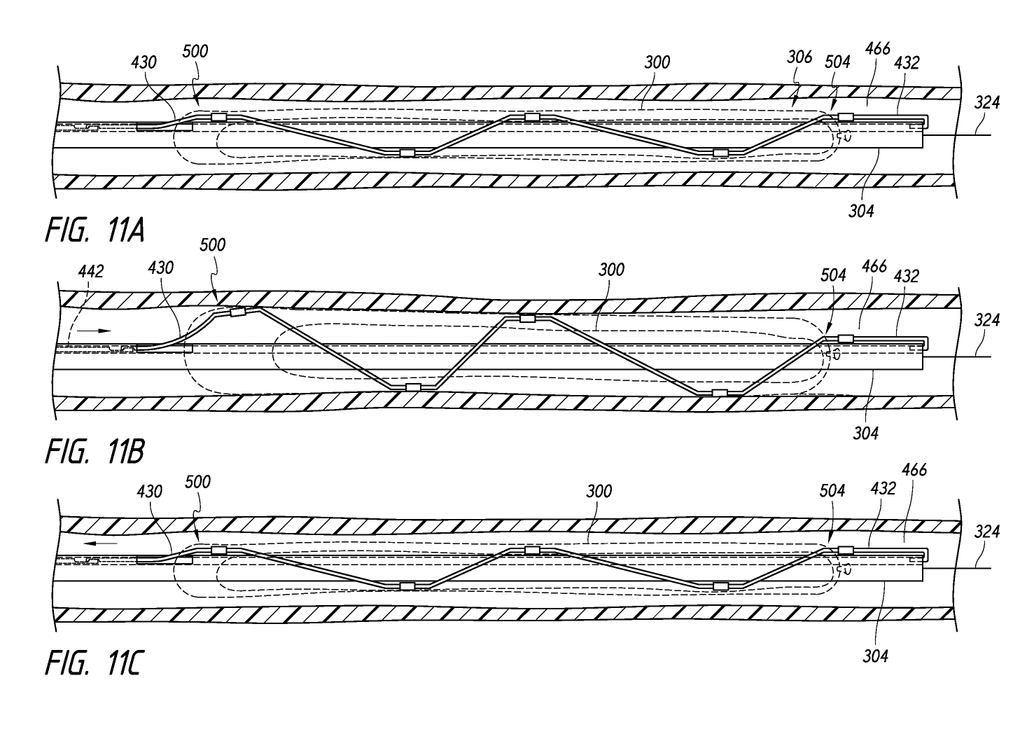

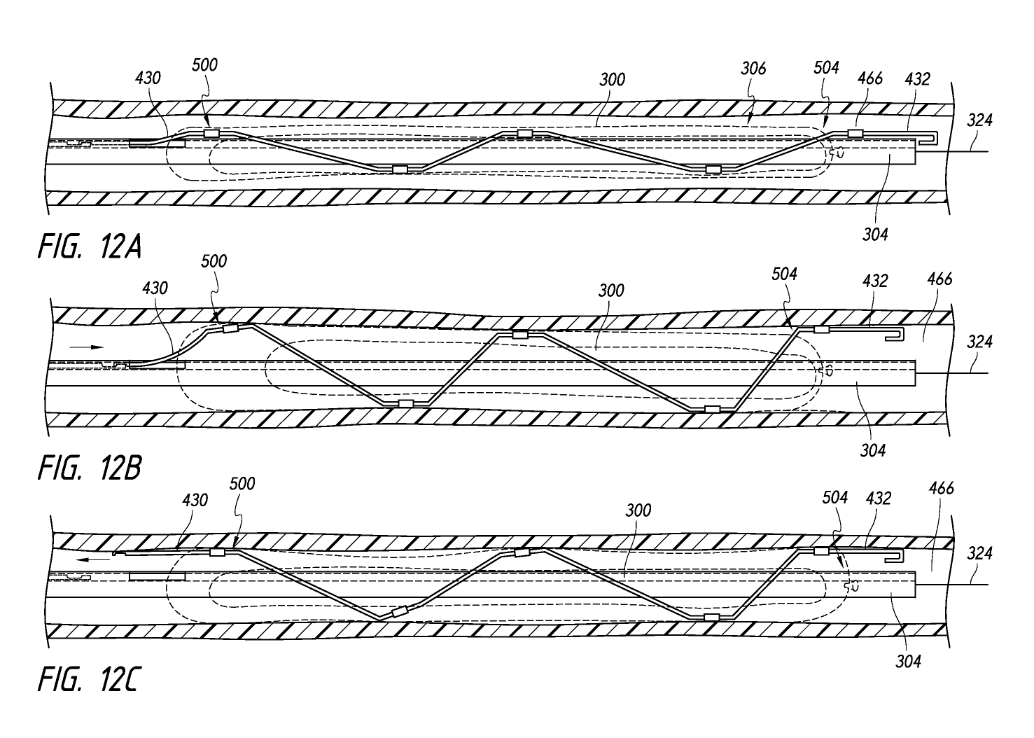

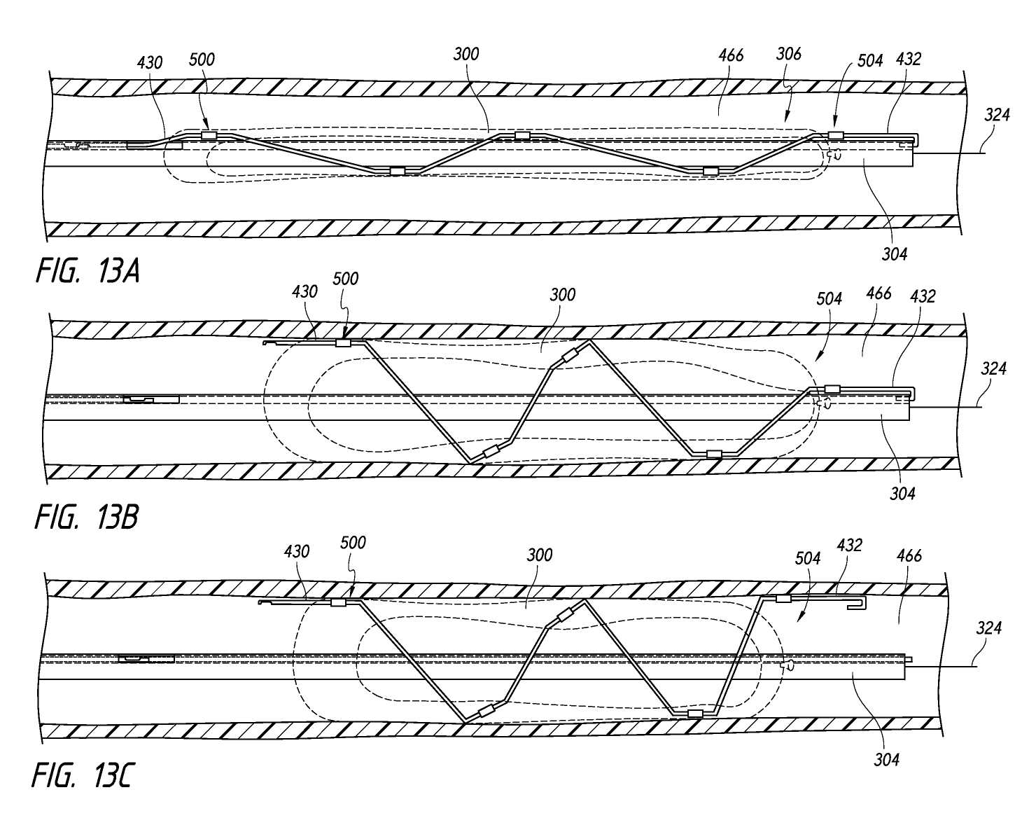

FIGS. 11A-13C show schematic illustrations of procedures using an implant carrier assembly or delivery system 306 carrying the implant 300 within a body lumen 466, according to some embodiments. FIGS. 11A-11C are schematic illustrations of stages in which the implant carrier assembly of FIG. 2 can expand and collapse an implant carried thereon within a body lumen, according to some embodiments. FIGS. 12A-12C are schematic illustrations of stages in which the implant carrier assembly of FIG. 2 releases an implant into a body lumen, releasing a distal end of the implant before releasing a proximal end of the implant, according to some embodiments. Finally, FIGS. 13A-13C are schematic illustrations of stages in which the implant carrier assembly of FIG. 2 releases an implant into a body lumen, releasing a proximal end of the implant before releasing a distal end of the implant, according to some embodiments.

In each of these procedures, the assembly 306 can be advanced over a guidewire 324 to a target area within a vessel 309. The assembly 306 can be advanced OTW without a guide catheter, thus enabling the assembly 306 to be advanced into smaller diameter vessels. The use of an OTW delivery mechanism can allow the assembly 306 to be more steerable and controllable. The guidewire 324 can comprise a diameter of between about 0.010 inches and about 0.025 inches, and in some embodiments, between about 0.013 inches and about 0.018 inches. Such sizes can facilitate the use of a smaller profile catheter and assembly, such as 5 Fr, 4 Fr, 3 Fr, or smaller.

However, in some embodiments, a guide catheter can be advanced over the guidewire 324 and the guidewire 324 can thereafter be removed and the implant carrier assembly 306 can be inserted into the guide catheter and advanced toward the target location. Once in position at the target location, when using a guide catheter, the guide catheter (not shown) and the implant carrier assembly 306 can be moved relative to each other such that the implant carrier assembly 306 begins to exit the distal end of the guide catheter.

When the assembly 306 is positioned at the target location, as illustrated in FIGS. 11A-11C, a proximal end 500 of the implant 300 can be expanded, as shown in FIG. 11B. As discussed above, this expansion can be performed by distally advancing the first actuation member 442, thus allowing the proximal coupling member 430 to be distally advanced. However, the longitudinal position of the second actuation member 452 (not shown) can be maintained relative to the assembly 306 such that a distal end 504 of the implant remains engaged and in a fixed position relative to the catheter 304. Thereafter, with unless the first actuation member 442 is permitted to disengage from the proximal coupling member 430 (by distally advancing the proximal coupling member 430 until it exits the aperture of the second actuator lumen 390 (not shown), the first actuation member 442 can be proximally retracted in order to collapse the diameter of the implant 300.

Thus, FIGS. 11A-11C demonstrate use of the assembly 306 in which the implant 300 can be selectively expanded and collapsed in order to temporarily block flow through a blood vessel. Various procedures can take advantage of the ability to temporarily block a blood vessel, as discussed in the present disclosure and other disclosures incorporated herein by reference.

In addition to being able to selectively or temporarily expand and collapse the implant 300, the assembly 306 can also be used to release the implant 300, with either the proximal end 500 or the distal end 504 being released first.

For example, FIGS. 12A-12C illustrate that the implant can be expanded by first disengaging the distal coupling member 432 from the catheter 304 such that the distal end 504 of the implant 300 begins to expand prior to expansion of the proximal end 500. Thereafter, as shown in FIG. 12B, the proximal end 500 of the implant 300 can be expanded by distally advancing the proximal coupling member 430 until becoming disengaged from the catheter 304 (shown in FIG. 12C).

Further, FIGS. 13A-13C illustrate that the implant can be expanded by first disengaging the proximal coupling member 430 from the catheter 304 such that the proximal end 500 of the implant 300 begins to expand prior to expansion of the distal end 504. Thereafter, as shown in FIG. 13B, the distal end 504 of the implant 300 can be expanded by distally advancing the distal coupling member 432 until becoming disengaged from the catheter 304 (shown in FIG. 13C).

### Radial Constraint Delivery Systems

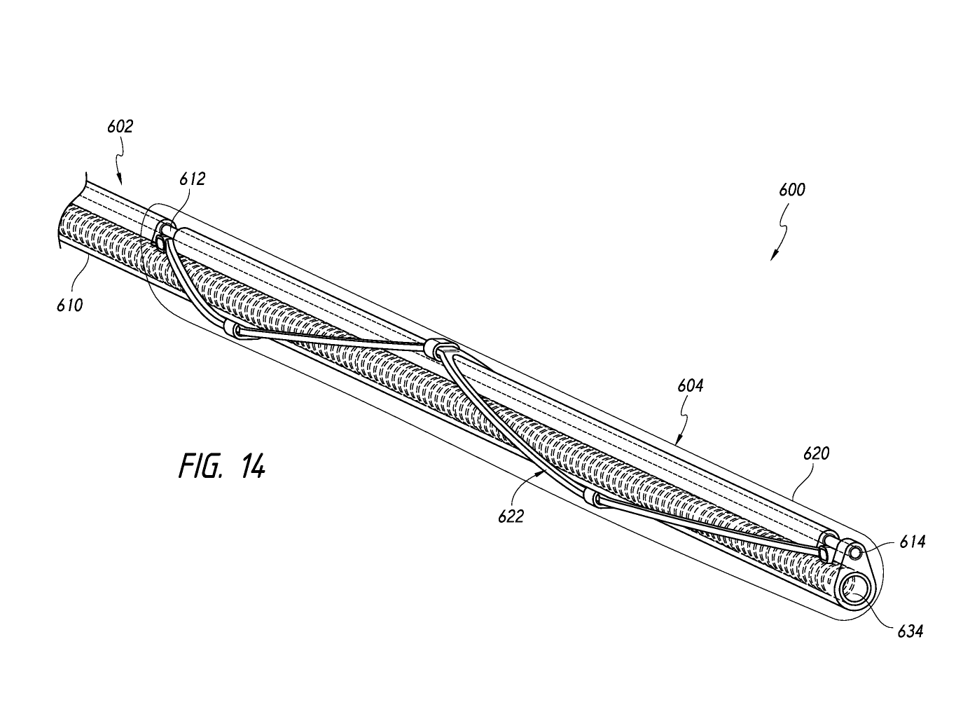

Referring now to FIGS. 14-19, some embodiments of an implant delivery system can provide a radial constraint or locking mechanism that can secure opposing ends or other portions of an implant frame relative to a catheter of the delivery system by radially overlapping the implant frame with an elongate wire extending along the length of the delivery system or enclosing the implant and catheter within a guide catheter. As illustrated in FIG. 14, and implant delivery system 600 can comprise the support member or assembly 602 that supports an implant 604 thereon. The support assembly 602 can comprise a catheter 610 and an actuation member 612 that extends within an actuator lumen 614 of the catheter 610. The implant 604 can comprise a cover member 620 and a support frame 622. The catheter 610 can also comprise a guidewire lumen 634. Further, the catheter 610 can be configured to include any of the features discussed above with respect to the catheter 304, such as the use of insert components and sizing of the lumens thereof.

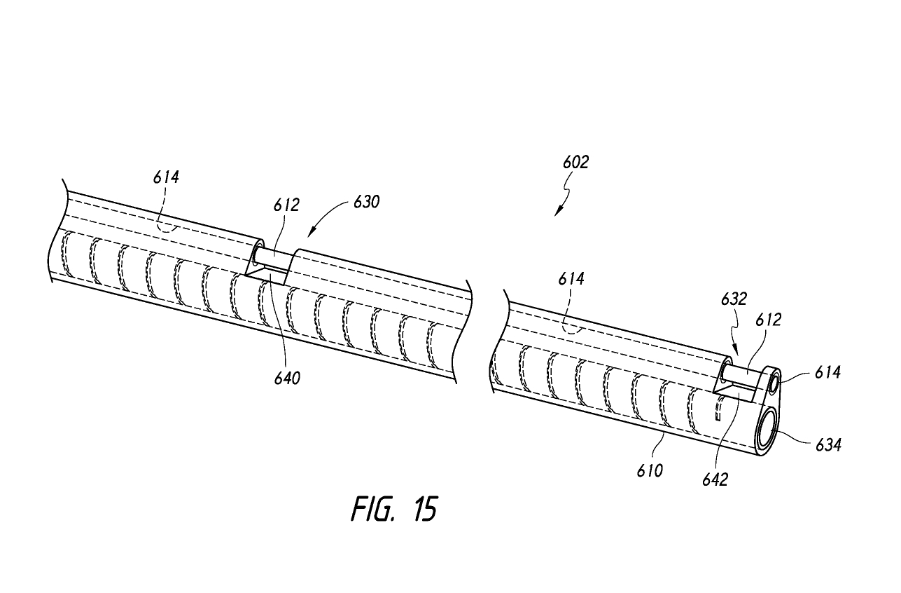

As shown in FIG. 15-17, the catheter 610 can comprise proximal and distal engagement sockets 630, 632. The proximal and distal engagement sockets 630, 632 can comprise a cutout regions or notches that extend radially into the catheter 610 through the actuator lumen 614. The proximal and distal engagement sockets 630, 632 extend to a sufficient depth such that a bottom surface 640, 642 of the respective ones of the proximal and distal engagement sockets 630, 632 is spaced apart from an outer surface of the actuation member 612 at a distance sufficient to accommodate a portion of the support frame 622.

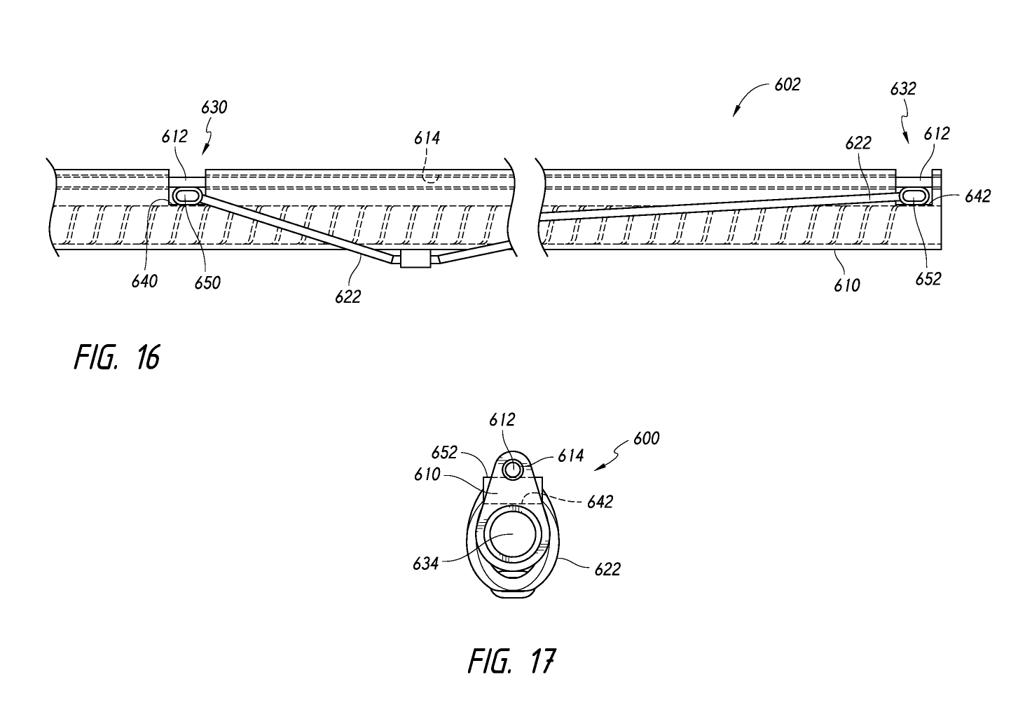

Thus, as shown FIG. 16-17, a proximal portion 650 of the frame 622 can be fitted into the proximal engagement socket 630 such that the actuation member 612 radially overlaps the proximal portion 650 and secures the proximal portion 650 within the proximal engagement socket 630. In accordance with some embodiments, the support frame 622 can comprise a series of loops that encircle the support assembly 602. Accordingly, when the actuation member 612 extends radially above or encloses the proximal portion of the frame 622 within the proximal engagement socket 630, the proximal portion 650 can be substantially fixed relative to the catheter 610. As also shown FIG. 16, a distal portion 652 of the frame 622 can be fitted into the distal engagement socket 632 such that the actuation member 612 radially overlaps the distal portion 652 and secures the distal portion 652 within the distal engagement socket 632. As noted above, in some embodiments, when the actuation member 612 extends radially above or encloses the proximal portion of the frame 622 within the distal engagement socket 632, the distal portion 652 can be substantially fixed relative to the catheter 610.

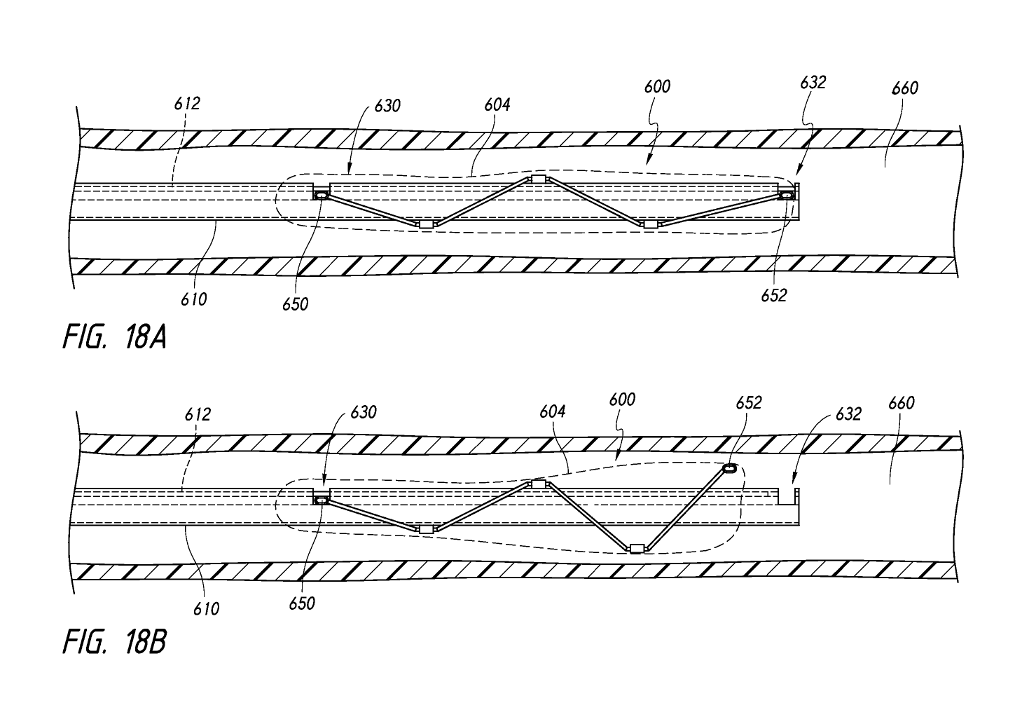

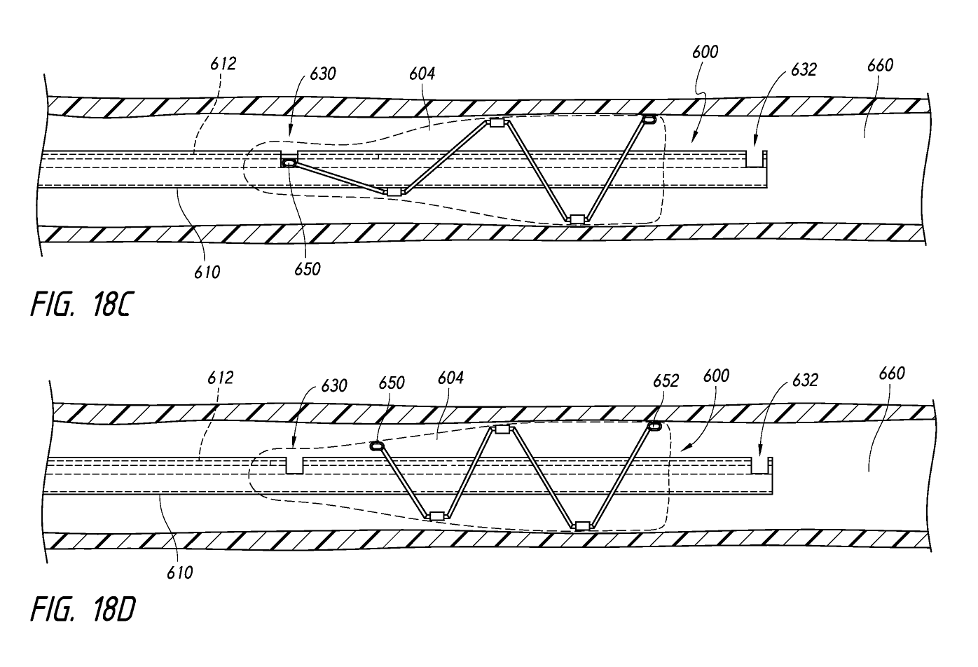

The implant 604 can be released from the support assembly 602 by proximally retracting the actuation member 612. Thus, as illustrated in FIGS. 18A-18D, when the implant delivery system 600 is positioned within a blood vessel lumen 660, proximal retraction of the actuation member 612 from over the distal portion 652 of the frame 622 can permit the distal portion 652 to exit or be released from the distal engagement socket 632 of the catheter 610 (shown in FIG. 18B). Further, continued proximal retraction of the actuation member 612 from over the proximal portion 650 of the frame 622 can permit the proximal portion 650 to exit or be released from the proximal engagement socket 630 (shown in FIG. 18D).

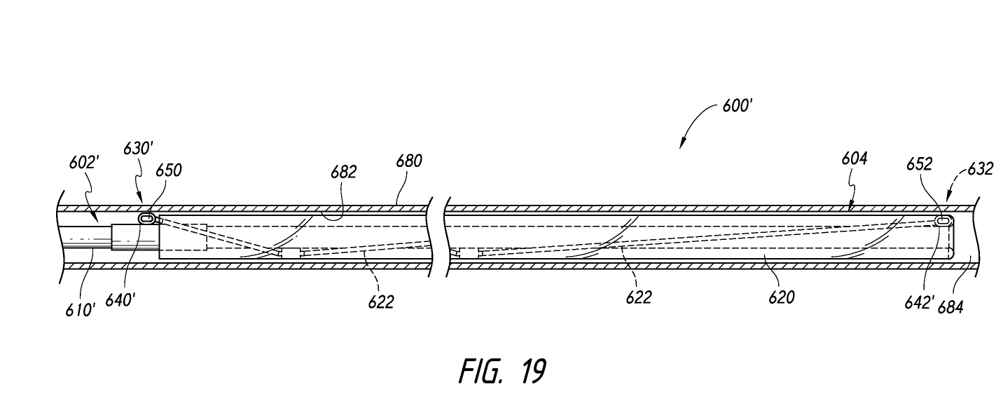

Referring now to FIG. 19, a modification of the implant delivery system 600 can be made to create a system 600′ that provides a radial constraint or locking mechanism to secure opposing ends of the implant 604 relative to the proximal and distal engagement sockets 630, 632 by enclosing the support assembly 602′ and the implant 604 within a guide catheter 680. Thus, the system 600′ can omit the actuation member 612 and the actuator lumen 614, and the guide catheter 680 can be configured such that an inner surface 682 of a lumen 684 of the guide catheter 680 creates a radial interference fit of the proximal portion 650 of the implant frame 622 between the inner surface 682 of the guide catheter 680 and the bottom surface 640′ of the proximal engagement socket 630, thereby longitudinally securing the proximal portion 650 relative to the catheter 610. Further, the inner surface 682 can also create a radial interference fit of the distal portion 652 of the implant frame 622 between the inner surface 682 and the bottom surface 642′ of the distal engagement socket 632.

The cross section of the catheter 610′ in the system 600′ can be much smaller than the cross section of the catheter 610 and the system 600 because of the omission of the actuation member 612 and the actuator lumen 614. The proximal and distal engagement sockets 630′, 632′ can also be shallower than the proximal and distal engagement socket 630, 632. For example, the proximal and distal engagement socket 630′, 632′ can be sufficiently deep such that up to about one half of the thickness of the proximal and distal portions 650, 652 can be exposed from the proximal and distal engagement sockets 630′, 632′.

Accordingly, in use, the implant 604 can be released from the system 600′ by proximally withdrawing the guide catheter 680 relative to the support assembly 602′ in order to permit the distal portion 652 of the implant frame 622 to exit the distal engagement socket 632′ as the distal portion of the implant 604 self-expands upon release from radial constraint by the guide catheter 680. Further proximal withdrawal of the guide catheter 680 relative to the support assembly 602′ can be performed in order to expose and permit the proximal portion 650 of the implant frame 622 to exit the proximal engagement socket 630′ as the proximal portion of the implant 604 self-expands upon release from radial constraint by the guide catheter 680.

### Pusher Component Delivery Systems

In accordance with yet other embodiments, another implant delivery system can be provided that radially encloses an implant frame within a lumen of a catheter to restrain radial expansion of the frame. A proximal portion of the implant can be engaged with an actuation component or member that can import longitudinal movement to the implant within the lumen. The implant can be expanded by exposing or releasing portions of the implant from within the lumen. However, until the actuation member disengages from the implant, the implant can be received or recaptured within the lumen by proximal retraction of the actuation member relative to the catheter.

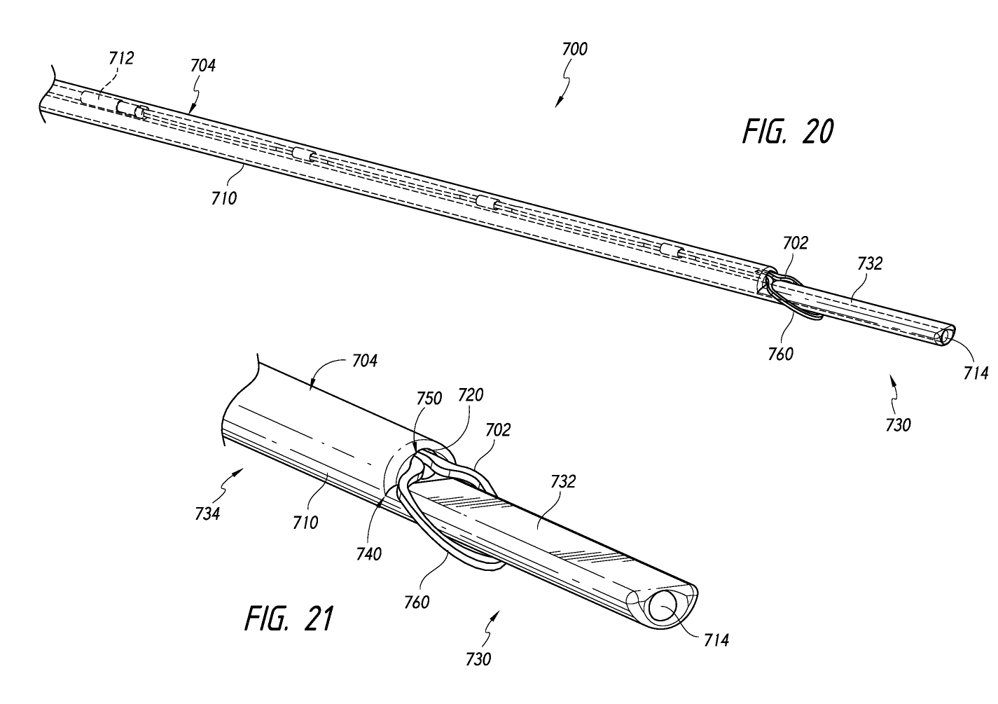

FIGS. 20-26 illustrates aspects of another embodiment of an implant delivery system 700 that can deliver an implant to a target region within a body lumen. The system 700 can comprise an implant 702 and a support assembly 704. The support assembly 704 can comprise a catheter 710 and an actuation member 712 that can be longitudinally advanced or withdrawn relative to the catheter 710 within an actuator lumen 720 of the catheter 710. The catheter 710 can also comprise a guidewire or fluid lumen 714. In any of the embodiments disclosed herein, the guidewire lumen can also be used to inject a fluid into the body lumen.

As shown in FIGS. 20-22, the catheter 710 can comprise a distal portion 730 having a tapered distal section 732. The tapered section 732 can comprise a cross-sectional profile that is less than a cross-sectional profile of a main section 734 of the catheter 710. The cross-sectional profile of the main section 734 of the catheter 710 can enclose both the actuator lumen 720 and the guidewire lumen 714. However, in some embodiments, the actuator lumen 720 can terminate at an intersection 740 of the tapered section 732 and the main section 734 of the catheter 710. The cross section of the catheter 710 can then step down from the cross section of the main section 734 to the reduced profile cross-section of the tapered section 732. Thus, the cross-sectional profile of the tapered section 732 can be configured to enclose the guidewire lumen 714, but not the actuator lumen 720.

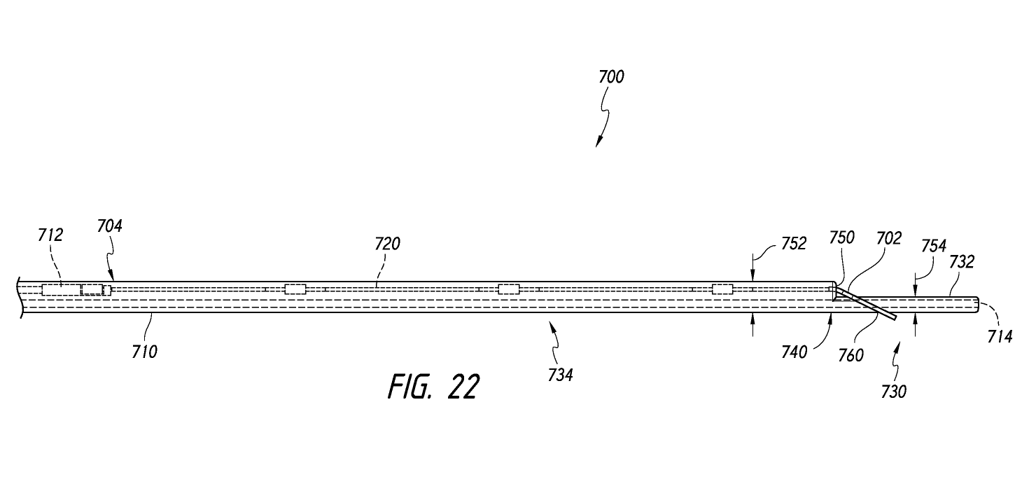

Further, the actuator lumen 720 can comprise an aperture 750 that opens to the tapered section 732. As noted above, and as illustrated in FIG. 22, the cross-sectional profile 752 of the catheter main section 734 is greater than a cross-sectional profile 754 of the catheter tapered section 732. Thus, the orientation of the aperture 750 relative to the tapered section 732 (and within the cross-sectional profile 752 of the main section 734) can permit the implant 702 to be distally advanced out of the catheter 710 without catching frictionally engaging the wall of the body lumen during release of the implant 702.

As also illustrated in FIGS. 20-22, the implant 702 can comprise a distal loop 760 that can extend outside of the actuator lumen 720 and be looped around the tapered section 732. The placement of the distal loop 760 around the tapered section 732 can provide a proximal stop that limits proximal movement of the implant 702 and actuation member 712 relative to the catheter 710. Further, during initial expansion of the implant 702, the distal loop 760 can be advantageously expanded into the body lumen without any interference from the tapered section 732 or any portion of the catheter 710. Further advantages, including the use of a cover member or membrane on the implant 702, are disclosed herein and referenced in the following discussion of the release and expansion of the implant 702 into a body lumen.

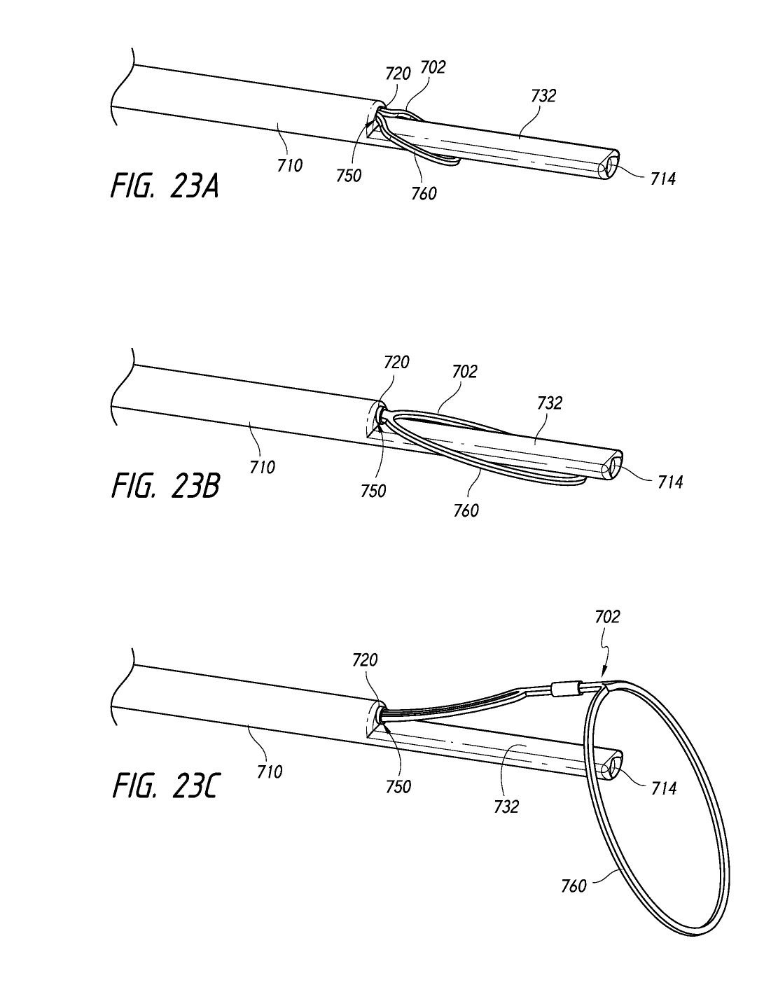

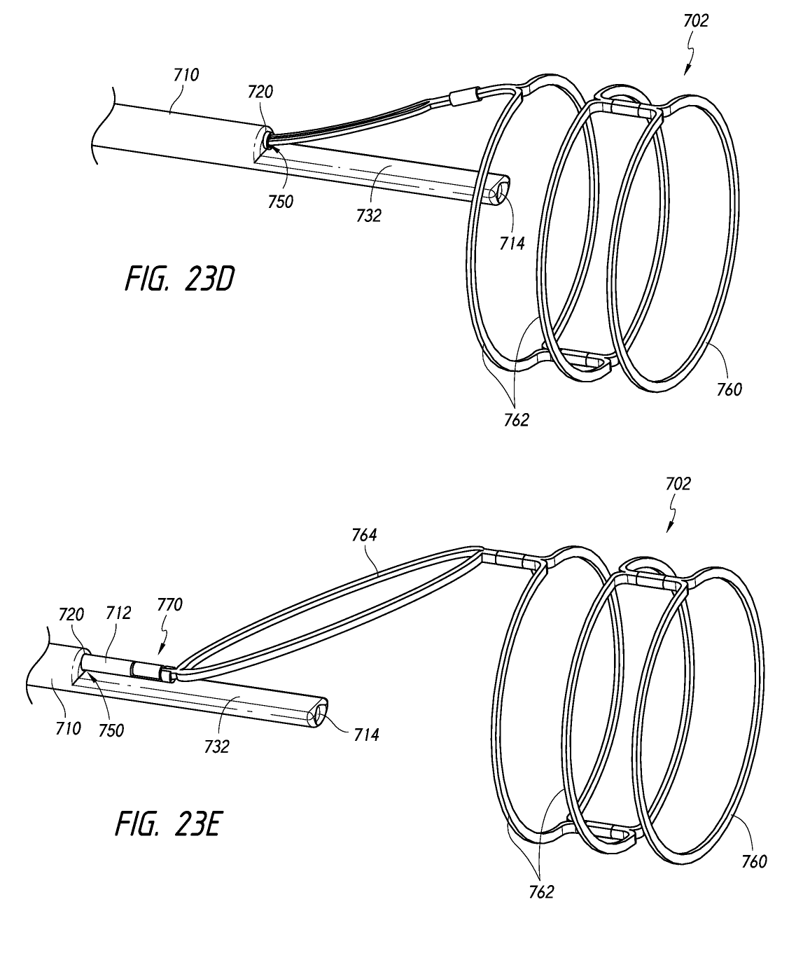

FIGS. 23A-23E illustrate aspects of the procedure for releasing the implant 702 from the implant delivery assembly 700. As shown in FIGS. 23A-23C, as the implant 702 is initially distally advanced from the aperture 750, the distal loop 760 becomes fully exposed and begins to expand radially. Continued distal advancement of the actuation member 712 permit additional loops 762 of the implant 702 to be exposed and begins expanding radially, as illustrated in FIG. 23D. Finally, as illustrated in FIG. 23E, a proximal loop 764 of the implant 702 can be exposed and begin expanding radially. The implant 702 can comprise a proximal coupling member 770 that can engage with a distal engagement component 772 of the actuation member 712. When the proximal coupling member 770 is fully exposed from within the lumen 720, the proximal coupling member 770 can disengage from the distal engagement component 772.

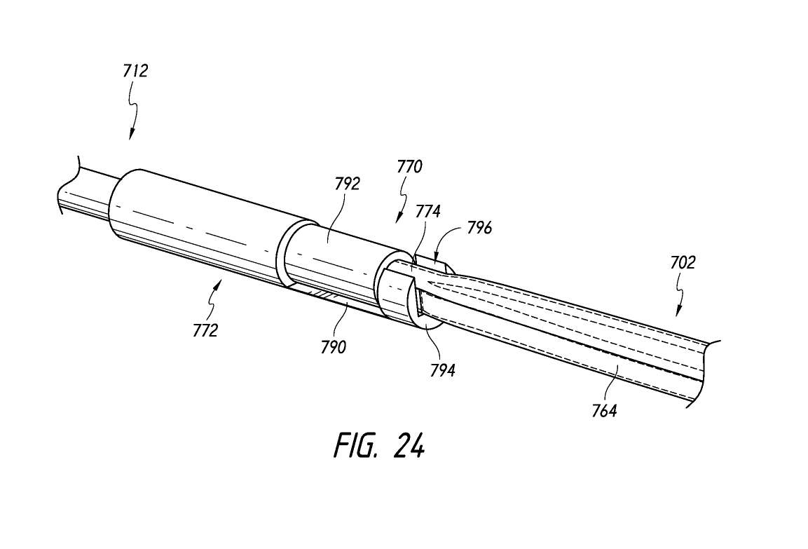

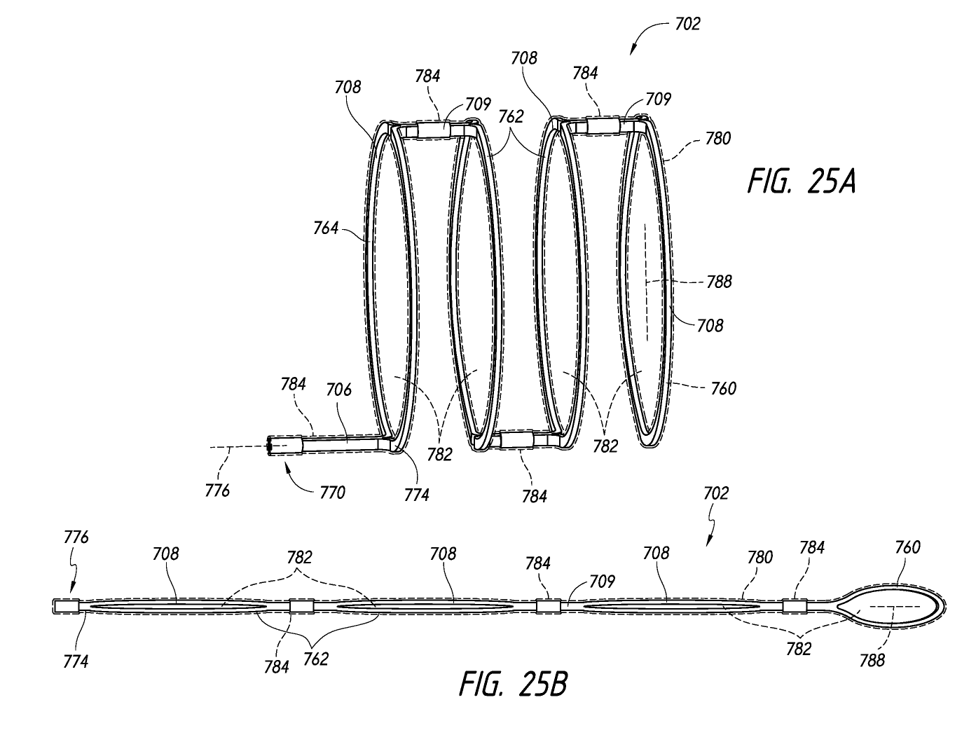

For example, the proximal coupling member 770 can be biased in a direction radially away from the tapered section 732. In some embodiments, the proximal loop 764 can comprise an elbow portion 774 (shown in FIGS. 24 and 25A) that can urge the proximal coupling member 770 to move toward and orientation that is generally transverse relative to the proximal loop 764. As shown in FIG. 25A, in its expanded or default configuration, the proximal coupling member 770 can extend along an axis 776 that is transverse or perpendicular relative to a plane (not shown) through which the proximal loop 764 extends. Thus, when released from within the lumen 720 (wherein the proximal coupling member 770 and the proximal loop 764 both extend substantially straight relative to each other, as shown in FIGS. 20, 22, 24, and 25B), the proximal coupling member 770 can tend to rebound toward its default configuration in which the proximal coupling member 770 moves towards a transverse orientation relative to the proximal loop 764 (as shown in FIG. 25A).

As shown in FIG. 24, the distal engagement component 772 of the actuation member 712 can be configured to comprise a recess and/or protrusion that can engage with a corresponding recess and/or protrusion of the proximal coupling member 770 of the implant 702. The distal engagement component 772 can comprise a recess 790 into which a bracket 792 of the proximal coupling member 770 can be received. Further, the distal engagement component 772 can comprise a stop member or shoulder 794 that can comprise an opening 796 where through the elbow portion 774 can pass. This configuration can permit the distal engagement component 772, the proximal coupling member 770, the actuation member 712, and the implant 702 to fit within a minimal diameter cross-sectional profile, such as that of the actuator lumen 720.

In addition, in some embodiments such as that illustrated in FIGS. 25A and 25B, the implant 702 can comprise a cover component 780 that extends around the implant frame 706. In some embodiments, the cover component 780 can comprise a tubular sock configuration that has an open end adjacent to the proximal coupling member 770 and a closed end that surrounds the distal loop 760. For example, the cover component 780 can comprise ePTFE tubing. The frame 706 of the implant 702 can be disposed within the cover component 780.

As discussed above with respect to FIGS. 3-4C, the frame 706 of the implant 702 can also comprise a plurality of support elements, loops, or hoops 708 that can be interconnected via backbone components or links 709. The loops 708 of the frame 706 can be generally circular and may be formed as loops or hoops. However, the loops 708 can be formed in any of a variety of shapes, including square, triangle, rectangle, oval, or other polygons (having five, six, seven, eight, nine, or more sides). Additionally, similar to the embodiment illustrated in FIGS. 3-4C, at least one of the hoops 708 can have an expanded or outer diameter different than one or more of the other hoops 708.

The cover component 780 can comprise one or more selected portions 782 that preferentially stretch to a diameter substantially greater than a non-stretched diameter. The selected portions 782 can correspond to the locations of the cover component 780 where the expandable loops 760, 762, 764 of the implant 702 are positioned within the cover component 780.

In some embodiments, the selected portions 782 can be expandable to a diameter that is between about 6 to about 30 times the size of the unexpanded diameter of the selected portions 782. In some embodiments, the selected portions 782 can be expandable to a diameter that is between about 8 to about 25 times the size of the unexpanded diameter of the selected portions 782. Further, the selected portions 782 can be expandable to a diameter that is between about 10 to about 20 times the size of the unexpanded diameter of the selected portions 782. For example, if the unexpanded diameter is about 0.005 inches, the selected portion 782 can expand to 0.030 inches or greater (e.g., to sizes such as 2 Fr, 3 Fr, 4 Fr, 5 Fr, 6 Fr, 7 Fr, 8 Fr, 9 Fr, 10 Fr, or greater. Additionally, in accordance with some embodiments, the selected portions 782 can be configured to expand such that each of the selected portions 782 expands to substantially the same expanded diameter. However, in some embodiments, a give selected portion can be configured to expand to a different expanded diameter than one or more of the other selected portions such that the cover component is able to expand to varying diameters, thereby closely approximating an expanded profile of an implant frame that has at least one loop having a diameter that is greater or smaller than a diameter of one or more of the other loops of the implant frame. In such embodiments, the cover component can closely fit over each of the loops 708 of the implant frame while permitting expansion of the loops 708 to different diameters.

Additionally, in some embodiments, the selected portions 782 can be pre-stretched to enable the implant 702 to expand more easily during deployment and to provide more reliable occlusion. Other selected or secondary portions 784 of the cover component 780, which can extend around or enclose the interconnecting links 709 (straight and/or round) can be non-expanding and/or remain or be left in an unstretched or “as manufactured” shape that permits a tight fit of the cover component 780 around the links 709. Thus, although the material of the secondary portions 784 may theoretically be expandable, the secondary portions 784 can remain in a non-expanded state, thereby closely fitting around the links 709, which can aid in securing the cover component 780 relative to the implant frame 706 without requiring any coupling device or process to secure the cover component 780 after releasing the implant 702 into the vessel.

Further, in some embodiments, the cover component 780 can have a variable diameter in the collapsed configuration, such that the selected portions 782 have a larger average diameter than the other secondary portions 784 of the cover component 780. For example, the selected portions 782 can be configured such that the cover component 780 is folded onto itself at the selected portions 782 (in order to maintain a low delivery profile). In such embodiments, during expansion of the loops 708, the one or more folds of material of the selected portions 782 can unfurl, reducing any initial restriction on expansion of the loops 708 (allowing the loops 708 to expand more quickly, thereby reducing the force required to initially expand the selected portions 782). The loops 708 of the frame 706 can thereby expand into the selected portions 782, which thus function as preformed pockets or widened sections of the cover component 780, facilitating rapid expansion and secure anchoring the frame 706 within the cover component 780.

Thus, the cover component 780 can attain a close fit along the entire length of the implant frame whether the implant 702 is in the collapsed or expanded configuration.

Further, the cover component 780 can optionally comprise a distal slit 788 that extends through the cover component 780 in the distalmost loop 760. Thus, in some embodiments, in a collapsed configuration, implant 702 can be positioned or mounted on the catheter 710 such that the tapered section 732 extends through the slit 788, as shown in FIG. 20. In some embodiments, as discussed below, the implant 702 can be used for deploying an embolic material to a target region, such as for cancer therapy, as disclosed in copending U.S. patent application Ser. No. 14/101,171, filed Dec. 9, 2013, the entirety of the disclosure of which is incorporated herein by reference. For example, a material, such as an embolic agent, can be delivered through the lumen 714 and through the slit 788 of the cover component 780 after the distalmost loop 760 is expanded within a vessel and before the remaining loops of the implant are fully expanded or released within the vessel. Thereafter, if released, the slit 788 can self-seal (based on backflow of some of the material, such as an adhesive ejected from the lumen 714) or otherwise substantially reduce or occlude flow through the lumen (especially in combination with the additional blockage performed by the other loops after the remainder of the implant 702 is expanded within the vessel).

However, some embodiments can also be provided that omit the distal slit 788 such that the distalmost loop 760 does not extend around the tapered section 732 (i.e., the tapered section does not extend through the distalmost loop 760). For example, the distalmost loop 760 can instead be at least partially or fully received or enclosed within the actuator lumen 720 during advancement of the system to the target region. When positioned at the target region, the implant 702 can be distally advanced from the lumen 720, as discussed above with respect to FIGS. 23C-23E, until the implant 702 is released into the vessel.

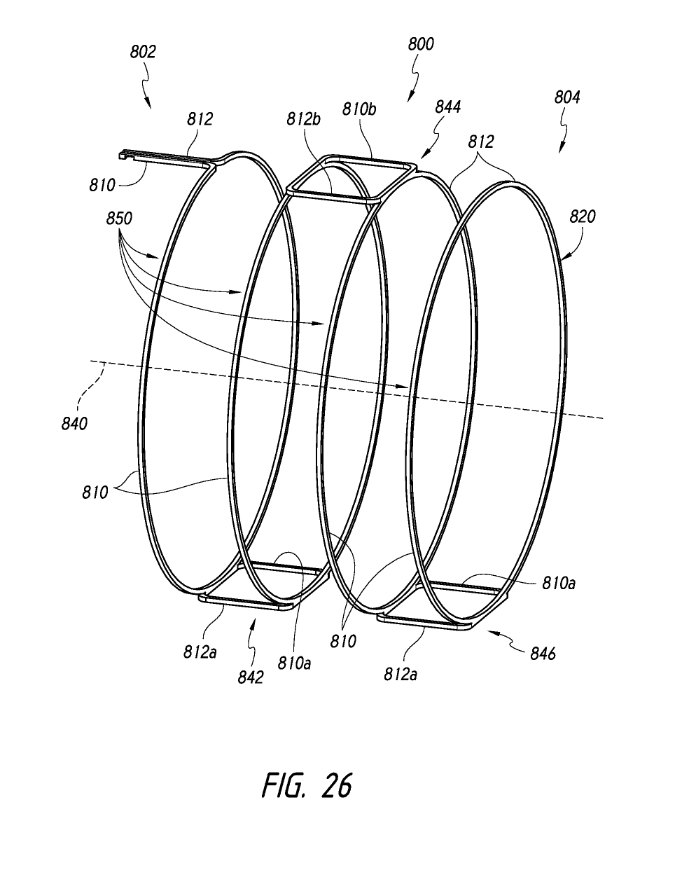

FIG. 26 is a perspective view of another implant device in an expanded state, according to some embodiments. In accordance with some embodiments, a medical implant 800 can form a frame including one or more dual wire loop features. As with the implant 702, the implant frame of the implant 800 can be configured similar to the implant 300 discussed above. However, the implant 800 can be formed from a single wire that extends from respective proximal ends and passes through a series of left and right loops until reaching a distal loop, which is approximately a midpoint for the wire. For example, the wire can comprise first and second halves 810, 812 that be configured to have opposite (clockwise and counterclockwise) directions of winding, and the halves 810, 812 can be connected to form full loops.

In some embodiments, the first and second halves 810, 812 can be generally mirror images of each other along a longitudinal center plane (extending through the central axis 840) of the frame 800, as illustrated in FIG. 26. The first and second halves 810, 812 extend from a first end 802 to a second and 804 of the frame 800. Additional first and second halves are joined together at a distalmost loop 820 of the frame 800.

The first and second halves 810, 812 may extend axially along axial portions 810a, 810b, 812a, and 812b. The axial portions 810a, 812a may be radially opposite the axial portions 810b, 812b across the central axis 840. Along the axial portions 810a, 812a, the first and second halves 810, 812 can overlap, cross, and/or contact each other. The first and second halves 810, 812 may be moveable relative to each other at overlapping sections 842, 844, 846. This overlap and free movement of the first and second halves 810, 812 at the overlapping sections 842, 844, 846 can allow the implant 800 to expand more readily and provide greater apposition of the implant 800 against the lumen wall. The amount or arc length of overlap between the first and second halves 810, 812 at the overlapping sections 842, 844, 846 can be between about 2% to about 40% of the circumference of the implant 800, or in some embodiments, between about 5% and about 20% of the circumference, or between about 10% and about 15% of the circumference.

The first and second halves 810, 812 may extend circumferentially along circumferential portions 850. The circumferential portions 850 may be radially opposite the circumferential portions of the other one of the first and second halves 810, 812 across or about the central axis 840. Each of the circumferential portions 850 may extend from an axial portion 810a, 812a to an axial portion 810b, 812b, radially opposite the axial portion 810a, 812a.

Thus, the interconnections of the first and second halves 810, 812 can lie substantially in a common plane. However, the first and second halves 810, 812 can also form interconnections that are not mirror images or that do not lie in a common plane. For example, in embodiments in which the frame 800 defines a generally tubular shape, the interconnections can be located at different and varied circumferential locations. For example, the interconnections can be distributed across one, two, three, four, five, or more circumferential locations. The pattern can be a repeating pattern or randomized, which can provide a desired flexibility or strength characteristics for the frame.

Additionally, the first and second halves 810, 812 can be of a common gauge or can have different gauges, in order to impart a desired strength characteristics.

Additionally, as illustrated above and in FIG. 26, as with various embodiments discussed herein, the frame 800 can be held in a generally linear or straight configuration within a lumen of a catheter. For example, ends 802, 804 of the frame 800 can be pulled or separated which can create tension between the ends 802, 804 to allow the frame 800, when deployed, to return an expanded shape consisting of one or more loops, as illustrated in FIG. 26, as the frame 800 exits from a distal end of the catheter.

### Valve Mechanisms

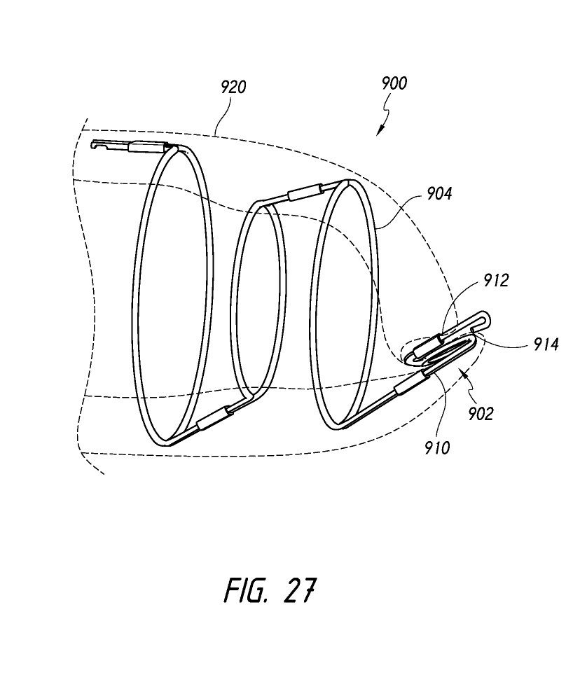

In accordance with some embodiments, one or more of the implants disclosed herein can be used for deploying an embolic material to a target region, such as for cancer therapy, as disclosed in copending U.S. patent application Ser. No. 14/101,171, filed Dec. 9, 2013, the entirety of the disclosure of which is incorporated herein by reference. For example, some embodiments can be used to perform such methods by optionally incorporating one or more valve mechanisms, such as those illustrated in FIGS. 27-30B. The valve mechanism can be a mid and/or distal valve coupled to the implant, such as being integrated into a frame of the implant.

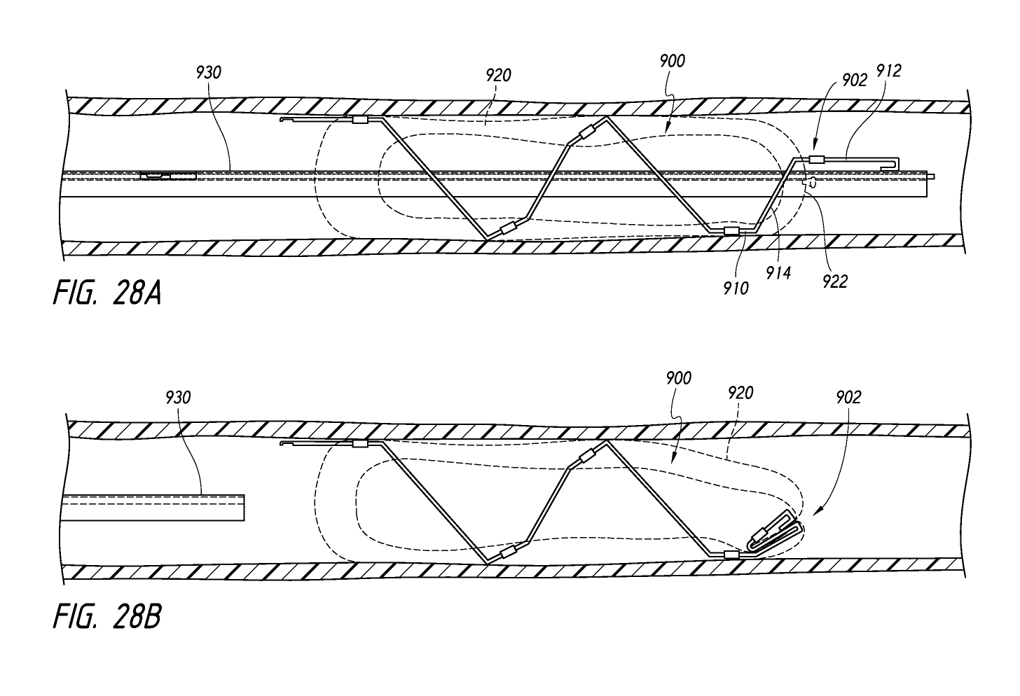

Referring to FIGS. 27-30B, some embodiments of the implant frame disclosed herein can comprise a valve mechanism that allows a portion of the implant frame to collapse, thus restricting flow through the implant. For example, FIGS. 27-28B illustrate an implant 900 that comprises a distal valve mechanism 902. The distal valve mechanism 902 can comprise a portion of a support frame 904 of the implant 900. As illustrated, and similar to the implant 300 discussed above, the implant frame 904 can comprise a series of loops that are interconnected by respective backbones. The distal valve mechanism 902 can comprise a first backbone 910 and a second backbone 912 that are both interconnected with a distalmost loop 914.

In accordance with some embodiments, as shown in the expanded or relaxed state in FIG. 27, the distalmost loop 914 can be biased toward the first backbone 910, and the second backbone 912 can be biased toward the distalmost loop 914. Thus, the first and second backbones 910, 912 can tend to collapse toward or lie against the distalmost loop 914 when the implant 900 is released into a body lumen. As such, the first and second backbones 910, 912 and the distalmost loop 914 can be biased towards a closed or collapsed position. Further, the implant 900 can comprise a cover component 920 that can be attached to the implant 900 in a manner disclosed herein or otherwise disclosed in Applicant's co-pending references listed herein and that are incorporated herein by reference.

For example, although the first backbone 910 and the distalmost loop 914 can be positioned within the cover component 920, the second backbone 912 can extend through a distal aperture 922 of the cover component 920 such that the cover component 920 becomes entangled within or between the first and second backbones 910, 912 and the distalmost loop 914 when the implant 900 is released from a support assembly 930, as illustrated in FIGS. 28A and 28B. The distal aperture 922 can be maintained in an open state when the support assembly 930 extends within a lumen of the implant 900 (e.g., before the support assembly 930 is proximally withdrawn from the distalmost loop 914). However, even when the support assembly 930 extends through the distalmost loop 914, the distalmost loop 914 can tend to undergo a torsional force that presses the distalmost loop 914 against the support assembly 930. Thus, when the support assembly 930 is withdrawn from the distalmost loop 914, the distalmost loop 914 move towards the collapsed position shown in FIGS. 27 and 28B.

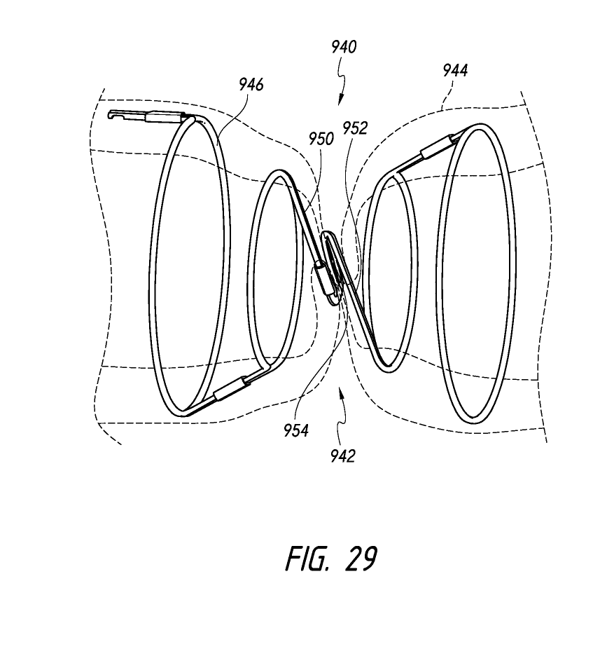

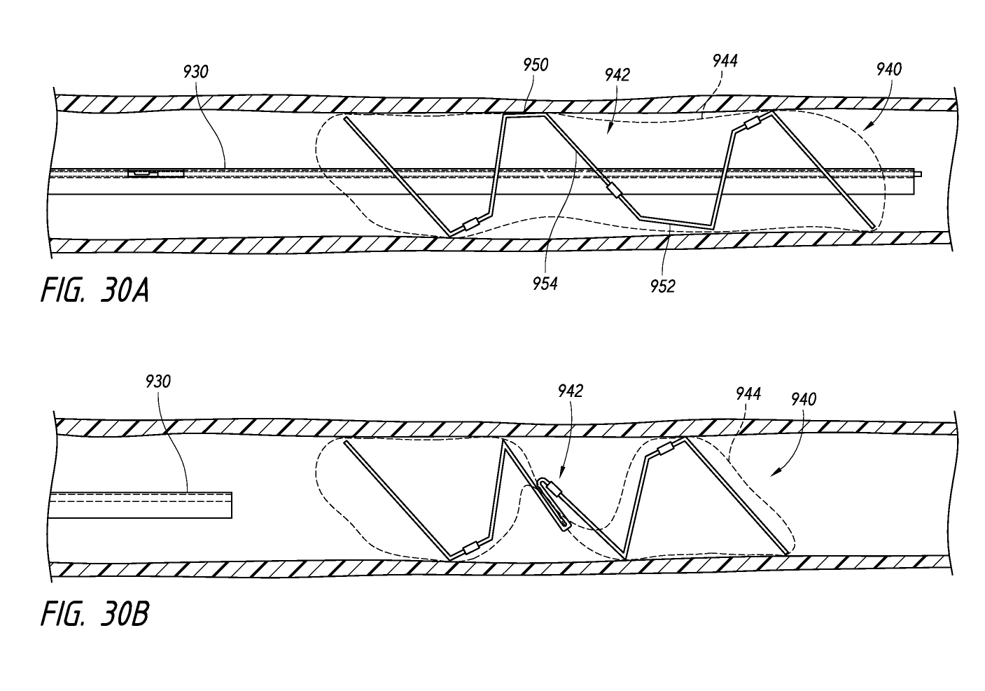

FIGS. 29-30B also illustrate an implant 940 that can comprise a central valve mechanism 942, a cover member 944, and a series of loops and backbones that form a support frame 946. The implant 940 can function similar to and include features similar to those discussed herein, including the implant 900 discussed in FIGS. 27-28B. However, the implant 940 comprises a valve mechanism 942 that is positioned in a central location of the implant. Nevertheless, the valve mechanism can comprise a first backbone member 950, a second backbone member 952, and a loop 954. The first and second backbone members 950, 952 and the loop 954 can be biased towards a closed or collapsed position that tend to close the lumen of the implant 940. Further, in some embodiments, the closure of the central valve mechanism 942 can entangle or collapse the cover member 944 if attached to the loop 954. Further, although illustrated as being smaller in size than adjacent loops, the loop 954 can be a similar size to adjacent loops and the cover member 944 can be attached thereto. FIGS. 30A and 30B illustrate the actuation of the central valve mechanism 942 when a carrier assembly 930 is withdrawn.

### Cover Component Features

In accordance with yet other embodiments, the implants disclosed herein can comprise a cover component that can be used to carry biocompatible medications or materials, such as hydrogels or embolic materials. Further, the implants disclosed herein can comprise cover components that extend around and/or within the support frames in a variety of ways.

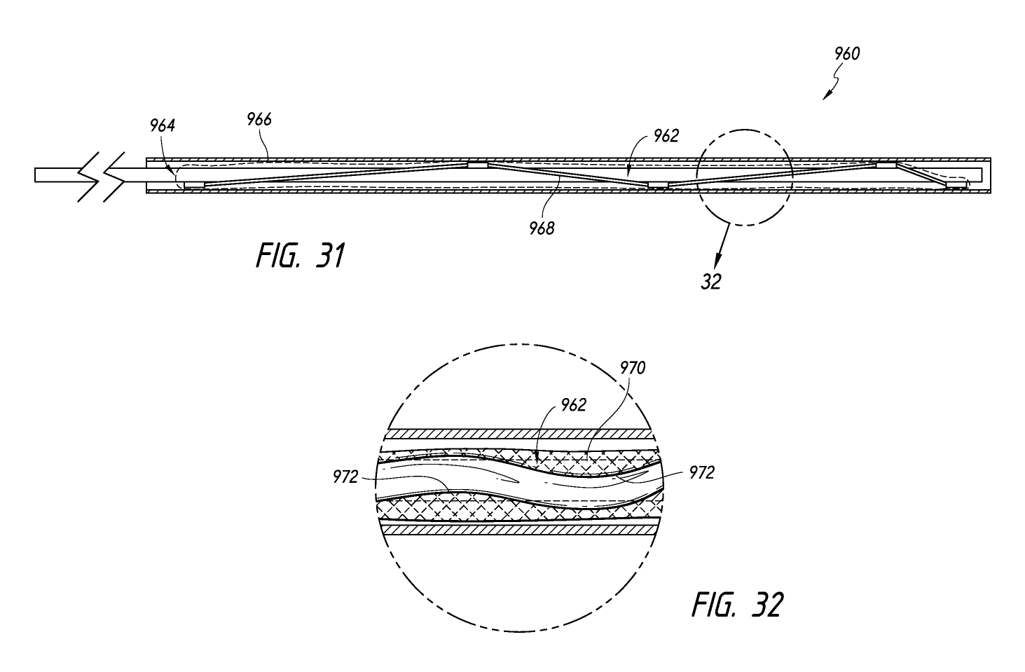

For example, FIGS. 31 and 32 illustrate an implant delivery system 960 that comprises an implant 962, a support assembly 964, and a guide catheter 966. The implant 962 can comprise a support frame 968 and a cover component 970. As shown in the enlarged view FIG. 32, the cover component 970 can be overlaid onto the collapsed support frame 968 such that a plurality of pockets 972 are formed by folding the cover component 970 onto itself. In accordance with some embodiments, excess material from the cover component 970 (which can be provided to allow sufficient material to allow the cover component 970 to expand from the collapsed cross-sectional profile to an expanded cross-sectional profile) can be folded onto itself to form folds or pockets. A material, such as medication, hydrogel, embolic materials, or other therapeutic agents can be disposed within the folds or pockets. The material can be placed into the folds or pockets after the folds are pockets have been formed or beforehand, with the fold or pocket being created by folding a portion of the cover component 970 over the material disposed on the cover component 970.

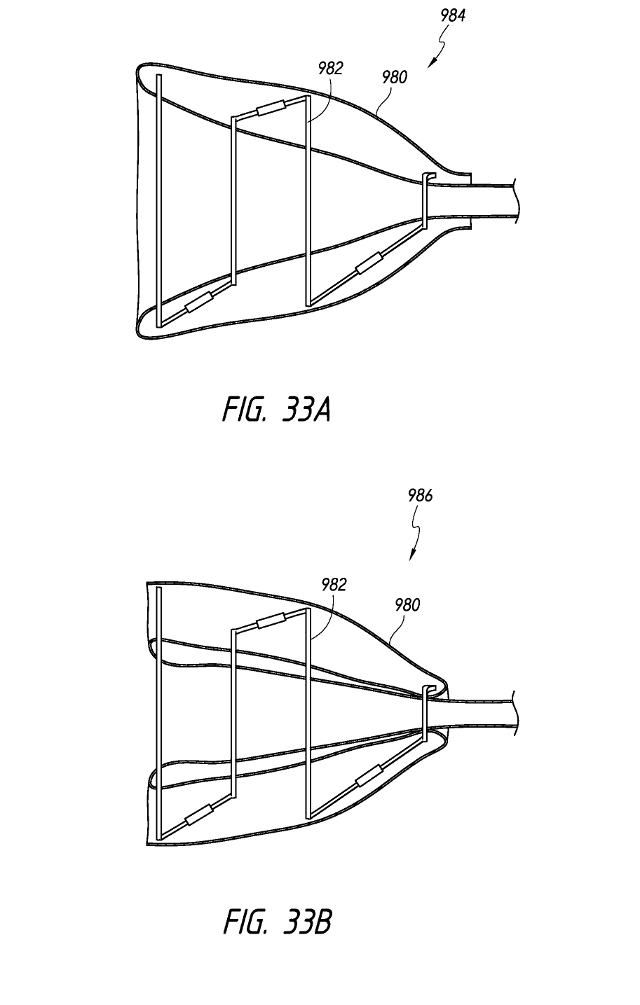

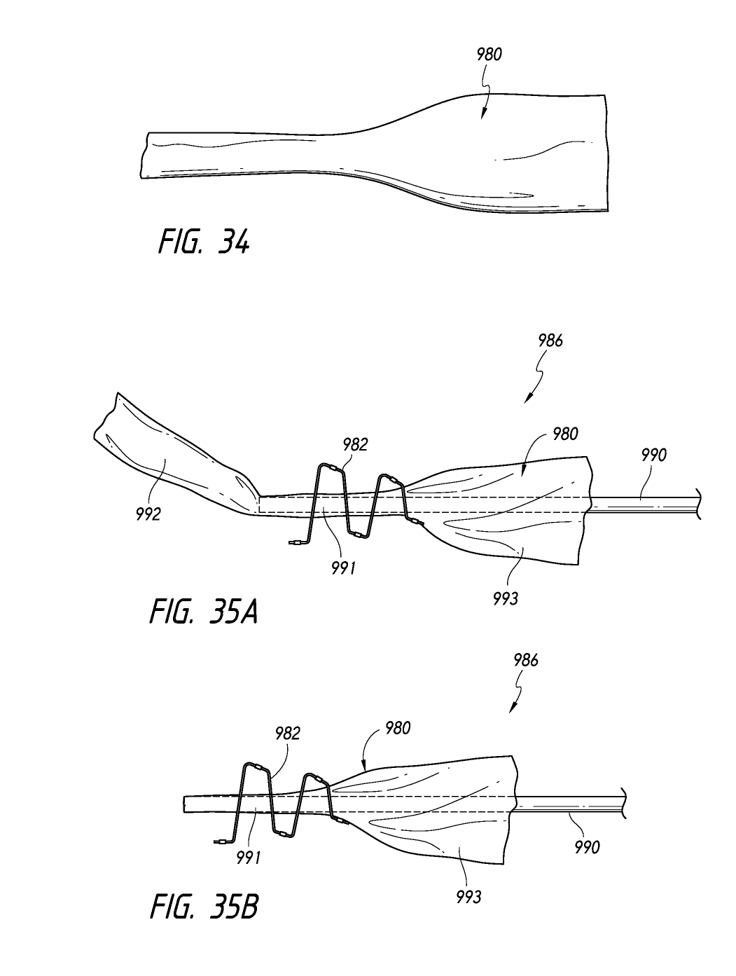

FIGS. 33A-33B illustrate example placement configurations of a cover component 980 relative to a support frame 982. In the embodiment shown in FIG. 33A, an implant 984 can be configured such that the cover component extends along an exterior and an interior of the frame 982. For example, the cover component 980 can comprise a tubular member (as generally shown in FIG. 34) that is placed over the frame 982 and inverted into a lumen of the frame 982 such that the frame 982 is positioned between the inner and outer tubular layers of the cover component 980. Thus, the implant 984 is configured such that ends of the tubular component are positioned at the same end of the frame 982 (shown in FIG. 33A as the distal end).

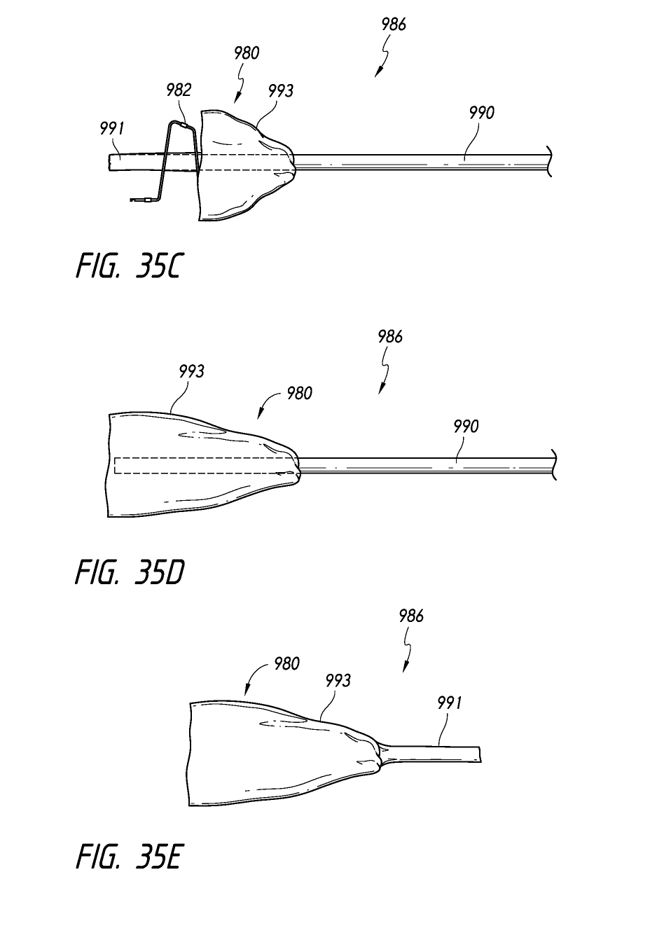

In the embodiment shown in FIG. 33B, an implant 986 can be configured such that the cover component 980, which can comprise a tubular member, extends over the frame 982 and is inverted twice within a lumen of the frame 982. Thus, along and exterior of the frame 982, the cover member 980 comprises a single layer and within a lumen of the frame, the cover member 980 comprises a dual layers. In accordance with an aspect of some embodiments, the cover members 980 can serve as a two-way valve structure that can block flow in both ways after deployment and removal of the guide wire and/or carrier assembly. The cover member 980 may be attached to a mid or distal portion of the implant. A procedure for forming the cover member configuration of the implant 986 shown in FIG. 33B is illustrated in FIGS. 35A-35E.

FIGS. 35A-35E illustrate steps in a process for manufacturing an implant device using the tubular cover member 980 of FIG. 34, according to some embodiments. First, the cover member 980 is positioned over a catheter 990, and the support frame 982 is thereafter also positioned onto the catheter 990 over the tubular cover member 980, such that the cover member 980 extends between the catheter 990 and the frame 982. As shown in FIG. 35A, the support frame 982 is positioned over a middle section 991 of the cover member 980. The middle section 991 of the cover member 980 is positioned between first and second sections 992, 993 of the cover member 980 that are of similar length or substantially equal lengths as the middle section 991. Thus, when the frame 982 is positioned over the middle section 991, the first section 992 can be inverted into a lumen of the catheter 990, as shown in FIG. 35B. Thereafter, as shown in FIGS. 35C-35E, the second section 993 can be everted over the frame 982. The second section 993 can enclose the frame 982 within the cover component 980. When the second section 993 has been everted, the implant 986 can be removed from the catheter 990 such that a portion of the first section 992 extends from an end of the implant 986.

### Further Stent Embodiments

In accordance with some embodiments, a reduced diameter or reduced cross-sectional profile stent or stent frame structure can be provided that allows a clinician to achieve immediate total occlusion of blood flow through peripheral vessels. In some embodiments, the stent or frame structure can have a nominal profile that is less than about five times the cross-sectional profile of the filament(s) or wire forming the stent or frame structure. For example, in some embodiments, the stent or frame structure can be formed using a single elongate wire that is drawn into a generally linear configuration and moved through a catheter lumen toward the target site. Some embodiments can comprise two or more elongate wires that can be drawn into generally elongate linear configurations. Accordingly, various embodiments can be provided in which the elongate wires are drawn into a minimum profile configuration that allows the stent to assume a collapsed configuration having a cross-sectional profile that allows the stent to be loaded and delivered using a very small gauge catheter.

In accordance with some embodiments, a medical implant can be provided that can be used in a variety of clinical applications, such as vessel occlusion, stenting, or other functions within a body vessel. The medical implant can comprise a first frame or anchoring component and one or more secondary components, second frame components, or occluding components.

The frame component can comprise one or more resilient members, such as wires, which can be drawn out into a delivery configuration in which the frame component is in a generally linear configuration and thereafter expand to an expanded state when released from a delivery device, such as a catheter.

Various embodiments of the frame component can be comprise one or more features, such as having a variable pitch, an alternating pitch, a laminated configuration, a consistent or constant pitch, and upright configuration, and/or a dual wire loop configuration, and/or any of the stent features discussed herein. Further, some embodiments of the frame component can be used with occlusive structures, valves, occlusive covers, fibrous membranes, and the like. The frame component can comprise a coil, laser cut tube, or braided wire structure. The frame component can be self-expanding or balloon expandable.

The secondary component, second frame component, or occluding component can be coupled to an end of the frame component. The secondary component, second frame component, or occluding component can also comprise one or more features, such as having a variable pitch, an alternating pitch, a laminated configuration, a consistent or constant pitch, and upright configuration, and/or a dual wire loop configuration, and/or any of the stent features discussed herein. Further, some embodiments of the secondary component can be used with occlusive structures, valves, occlusive covers, fibrous membranes, and the like. The secondary component can comprise a coil, laser cut tube, or braided wire structure. The secondary component can be self-expanding or balloon expandable.

The first and second frame members can incorporate other aspects of stents and stent systems disclosed in Applicant's co-pending patent applications: U.S. patent application Ser. No. 12/826,593, filed on Jun. 29, 2010 (086538-0012); U.S. patent application Ser. No. 13/367,338, filed on Feb. 6, 2012 (086538-0018); U.S. patent application Ser. No. 12/906,993, filed on Oct. 18, 2010 (086538-0014); U.S. patent application Ser. No. 13/828,974, filed on Mar. 14, 2013 (086538-0030); U.S. Patent App. No. 61/836,061, filed on Jun. 17, 2013 (086538-0038); U.S. patent application Ser. No. 14/044,794, filed on Oct. 2, 2013 (086538-0039); U.S. patent application Ser. No. 14/281,797, filed on May 19, 2014 (086538-0055); U.S. Patent App. No. 61/835,406, filed on Jun. 14, 2013 (086538-0032); U.S. Patent App. No. 61/904,376, filed on Nov. 14, 2013 (086538-0041); U.S. Patent App. No. 61/904,379, filed on Nov. 14, 2013 (086538-0043); U.S. Patent App. No. 61/835,461, filed on Jun. 14, 2013 (086538-0034); U.S. Patent App. No. 61/900,321, filed on Nov. 5, 2013 (086538-0040); U.S. patent application Ser. No. 14/101,171, filed on Dec. 9, 2013 (086538-0046); U.S. Patent App. No. 61/987,446, filed on May 1, 2014 (086538-0054); and U.S. patent application Ser. No. 14/304,868, filed on Jun. 13, 2014 (086538-0057), the entireties of which are incorporated herein by reference.

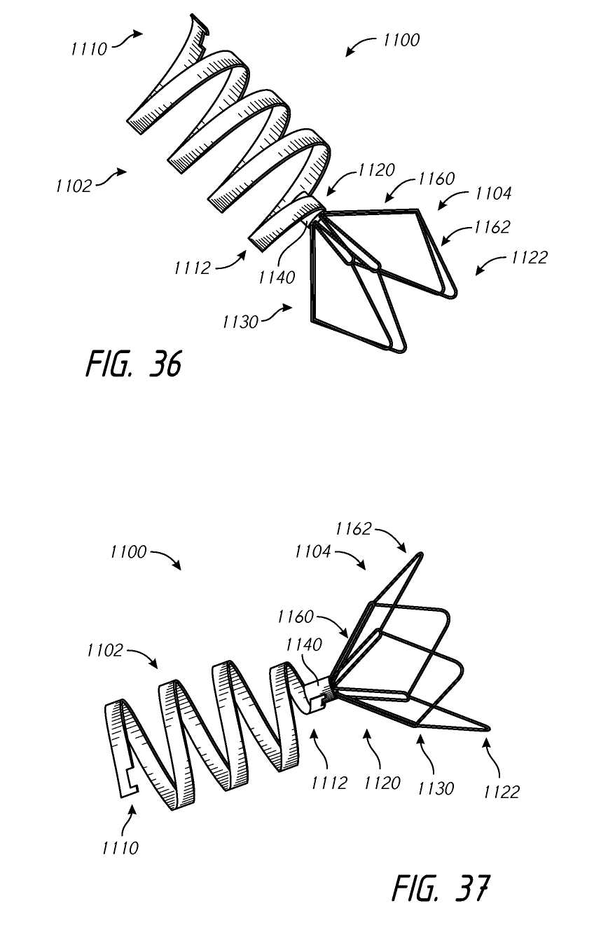

Referring now to the figures, FIGS. 36-38 illustrate an embodiment of an implant device 1100 that can be delivered to a target location for occluding a blood vessel or body lumen. The implant device 1100 can comprise a frame having a first frame component 1102 (e.g., a helical member) and a second frame component 1104 (e.g., an occlusive member). The helical member 1102 can comprise a proximal end portion 1110 and a distal end portion 1112. The occlusive member 1104 can comprise a proximal end portion 1120 and a distal end portion 1122.

The helical member 1102 can be configured to be supported or engaged by a distal end of a catheter. The helical member 1102 can comprise a flat coil following a helical path. However, the helical member 1102 can comprise other configurations, as discussed above.

The helical member 1102 can be used to anchor the occlusive member 1104 within the blood vessel or body lumen. In some embodiments, the helical member 1102 can have an axial width that is greater than its radial thickness. However, the dimensions and configuration of the helical member 1102 can vary, as noted in the above-referenced patent applications.

The helical member 1102 illustrated in FIGS. 36-38 can also be used in the devices illustrated in FIGS. 39-41. Additionally, any coupling means, which interconnects the helical member 1102 to the occlusive member, disclosed for one embodiment can also be used in any of these embodiments.

The occlusive member 1104 can comprise a plurality of support components 1130 that are radially expandable from a collapsed configuration to an expanded configuration. For example, a semi-collapsed configuration is illustrated in FIG. 36, and a semi-expanded configuration is illustrated FIG. 37.

The occlusive member 1104 can be interconnected with the helical member 1102. For example, in some embodiments, the device 1100 can comprise a coupling means, such as a coupling component or ring 1140 configured to interconnect the occlusive member 1104 with the helical member 1102. The coupling ring 1140 can be crimped onto the distal end portion 1112 of the helical member 1102 with the proximal ends of the support components 1130 interposed therebetween. Other means for coupling can also be used, such as chemical, adhesive, bonding, welding, or other mechanical means.

The support components 1130 can move between collapsed and expanded positions. For example, the occlusive member 1104 can be deflectable relative to the helical member 1102. For example, the support components 1130 can be deflectable or pivotable relative to the helical member 1102, such as relative to a longitudinal axis of the helical member 1102. Such embodiments are illustrated in FIGS. 36-41.

In some embodiments, as shown in FIGS. 36-37, the support components 1130 can comprise first and second portions 1160, 1162. The first portion 1160 can be deflectably coupled to the coupling ring 1140, and the second portion 1162 can be deflectably coupled to the first portion 1160 to move from the collapsed configuration to the expanded configuration. As shown in FIGS. 36-37, a proximal end of the second portion 1162 can be deflectably coupled to a distal end of the first portion 1160. For example, the proximal end of the second portion 1162 can be pivotably coupled to the distal end of the first portion 1160.

The first portion 1160 can comprise a plurality of elongate members. As shown FIGS. 36-37, the first portion 1160 can comprise a series of elongate members that include a loop at a distal end thereof. The second portion 1162 can comprise a wire that is formed in a circumferential loop. The wire of the second portion 1162 can comprise a series of peak sections and valley sections, as illustrated in FIGS. 36-37. The wire of the second portion 1162 can be interconnected with the elongate members of the first portion 1160. For example, the elongate members can be coupled at their distal ends to the valley sections of the wire of the second portion 1162.

Thus, in the embodiment illustrated in FIGS. 36-38, the second portion 1162 can be released from within the catheter and spring-loaded or biased such that the second portion 1162 self-expands toward the expanded configuration. In some embodiments, the first portion 1160 can also be spring-loaded or biased toward the expanded configuration to facilitate expansion of the occlusive member 1104.

The first and second portions 1160, 1162 can be coupled to each other by various means. For example, the first and second portions 1160, 1162 can be attached to each other threadably, adhesively, welded, or by mechanical means, such as a fastener or coupling. In some embodiments, the first and second portions 1160, 1162 can be pivotable relative to each other at the interconnection point. However, the first and second portions 1160, 1162 can also be rigidly attached to each other such that the first and second portions 1160, 1162 deflect relative to each other to move between the collapsed and expanded configurations.

In accordance with some embodiments, FIG. 39 illustrates an implant device 1200 that comprises the helical member 1102 and an occlusive member 1204. The helical member 1102 can be configured as noted above with respect to FIGS. 36-38 or as in any of the above-noted patent applications.

The occlusive member 1204 can comprise a plurality of support components or elongate members 1206. The support components 1206 can be interconnected by a plurality of linking portions 1208. The linking portions 1208 can extend between adjacent support components 1206, such as along a midpoint of the support components 1206 or at other locations along the support components 1206.

For example, the support components 1206 can be configured as elongate wires that are spring-loaded or biased toward an expanded configuration whose shape can be limited or constrained by the linking portions 1208. The linking portions 1208 can comprise a material that is flexible. In some embodiments, the linking portions 1208 can be substantially inelastic. However, the linking portions 1208 can also be elastically deformable. The length, location, and number of linking portions 1208 can determine the expanded configuration of the occlusive member 1204. Thus, the occlusive member 1204 can move towards the expanded configuration and assume a preset shape, as determined by the linking portions 1208.

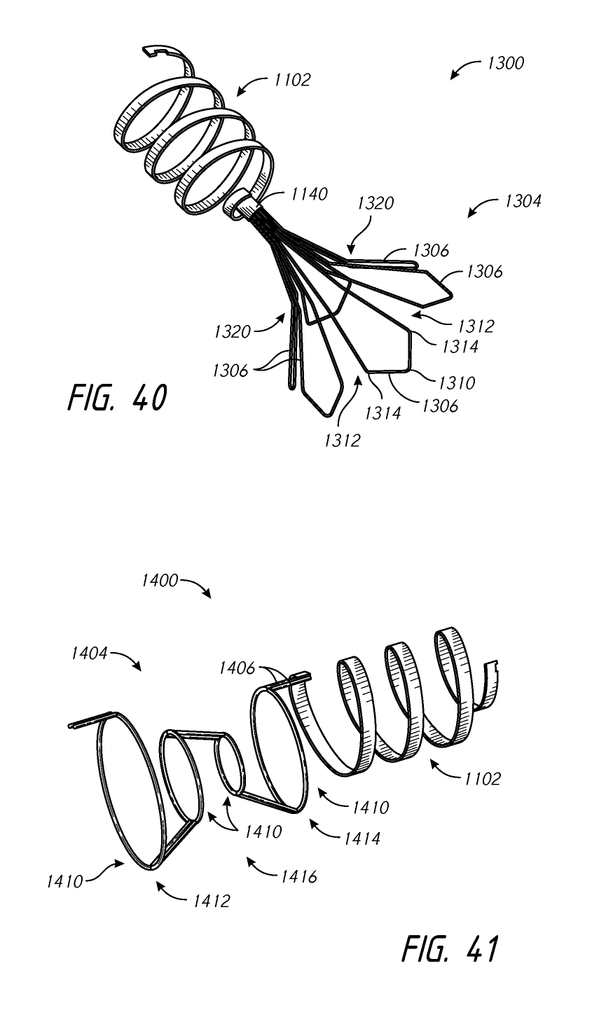

Referring now to FIG. 40, another embodiment is illustrated in which an occlusive implant 1300 comprises the helical member 1102 and an occlusive member 1304. The occlusive member 1304 can comprise a series of spring-loaded support components 1306 that are biased from a collapsed configuration toward an expanded configuration. The support components 1306 can each comprise a wire, which in some embodiments, can be formed in a loop, as illustrated in FIG. 40.

For example, the support components 1306 can be formed as a wire loop having a distal bend 1310 forming opposing loop halves 1312 and opposing lateral bends 1314 in the opposing loop halves 1312. In the collapsed configuration, the wire loop 1306 can be compressed such that the opposing loop halves 1312 are drawn together into a substantially linear configuration. When released and permitted to expand towards the expanded configuration, the wire loop 1306 can resiliently release to the expanded configuration as the distal bend 1310 and the opposing lateral bends 1314 revert to their preset, bended configuration.

For example, the distal bend 1310 can comprise a substantially 45° angle. However, the distal bend 1310 can be between about 30° and about 60° . Further, the opposing lateral bends 1314 comprise an obtuse angle. For example, the opposing lateral bends 1314 can comprise an angle of between about 90° and 120°.

In some embodiments, the support components 1306 can also comprise a radial bend 1320. For example, a distal portion of the support components 1306 can bend away from a longitudinal axis of the occlusive member 1304 at the radial bend 1320.

Thus, the support component 1306 can be spring-loaded or biased to expand from the collapsed configuration using one or more of the bends disclosed herein. Further, additional bends can also be made in the support components 1306 to facilitate shaping of the occlusive member 1304.

In accordance with yet another embodiment, FIG. 41 illustrates an embodiment of an occlusive implant 1400 that comprises the helical member 1102 and an occlusive member 1404. The occlusive member 1404 can comprise a pair of wires 1406 that comprise a preset shape and/or are coupled to each other such that the wires 1406, where can be moved from a collapsed configuration towards an expanded configuration. In the collapsed configuration, the wires 1406 can be substantially linearly arranged such that the wires 1406 can be drawn into a catheter lumen. However, when released from the catheter lumen, the wires 1406 can be biased towards the expanded configuration. In the expanded configuration, in some embodiments, sections of the wires 1406 can be separated from each other to form a plurality of support components 1410. For example, the occlusive member 1404 can be configured similar to the embodiments discussed above in FIGS. 3, 25A-27, and 29.

In some embodiments, the wires 1406 can have substantially mirror configurations. Thus, the wires 1406 can extend adjacent to each other in sections whereat the wires 1406 can be coupled to each other and separate into sections whereat the wires 1406 form the plurality of support components 1410.

Further, where the wires 1406 separate from each other, the wires 1406 can define support components 1410 in a variety of shapes and sizes. For example, the support components 1410 can define substantially circular shapes. The support components 1410 can also have different sizes and be spaced apart from each other along a longitudinal axis of the occlusive member 1404.

The expanded dimensions of the two intermediate components 1416 can be less than the expanded dimensions of the proximal and distal components 1412, 1414.

According to some embodiments, the expandable portion of the helical member can be entirely axially spaced apart from an expandable portion of the occlusive member.

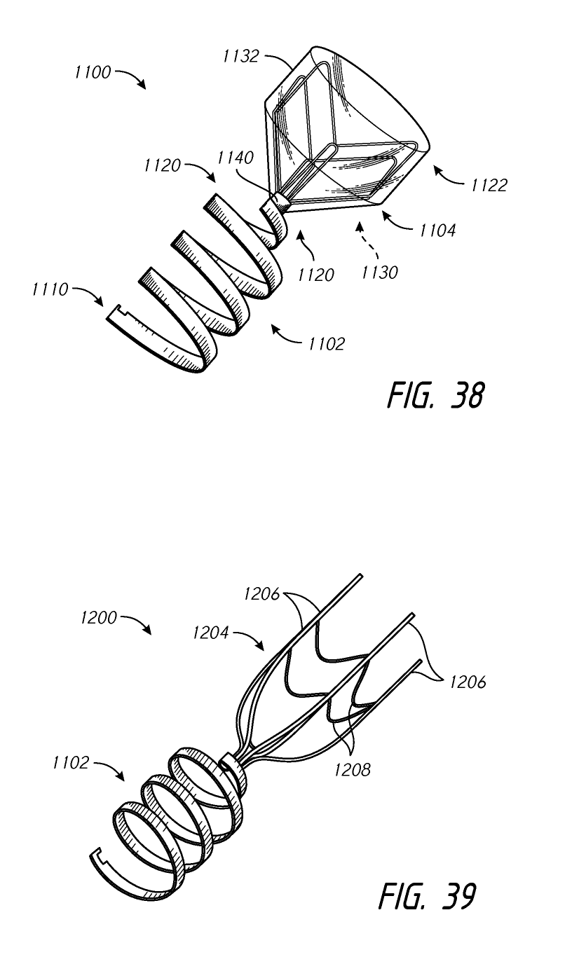

Additionally, FIG. 38 illustrates the use of an occlusive membrane or cover member 1132. Such a cover member 1132 can be used in any of the embodiments disclosed herein. The cover member can be attached to the device and extend at least partially along the occlusive member. The occlusive member can be positioned within the cover member. Further, the cover can be coupled to a coupling member that interconnects the helical member and the occlusive member.

The implant device discussed above with respect to FIGS. 36-41 can be implanted in a manner similar to that discussed above with respect to other embodiments. For example, the catheter can be advanced to a target location within a body vessel. The catheter can comprise a lumen in which the device is disposed. The device can be configured as any of the devices disclosed herein, which can comprise a helical member and an occlusive member coupled to a distal end of the helical member. Once the catheter is advanced to the target location, the device can be advanced out of the lumen to permit separate expansion of the helical member and the occlusive member. For example, the occlusive member can be expanded first and the helical member can be expanded second. Various other features, including use and/or expansion of a cover member can also be performed. The cover member can be coupled to the device and/or repositioned over the device.

In some embodiments, a length of the helical member or support frame may be between about 7 millimeters (mm) and about 9 mm. In some embodiments, the length of support frame may be less than about 7 mm or greater than about 9 mm. In some embodiments, the length of distal portion may be less than about 3 mm or greater than about 4 mm. In some embodiments, a diameter of the proximal portion and/or the middle portion may be between about 2 mm and about 10 mm. In some embodiments, the diameter of the proximal portion and/or the middle portion may be less than about 2 mm or greater than about 10 mm.

FIGS. 36-41 provide various embodiments of implant devices that provide peripheral vessel occlusion by delivery of radially expandable implant frames that achieve immediate total occlusion of blood flow. The implant devices can be implanted in vessels having a size of between about 3 mm to about 20 mm, with target delivery profile of about 3 Fr to about 5 Fr. The implant device can maintain acceptable blood flow through the blood vessel, while diverting flow away from diseased area or aneurysm.

Some embodiments of the device can also provide venous stenting in vessels having a size of between about 3 mm to about 16 mm, with target delivery profile of about 8 Fr or smaller. Deployment of the implant device can impart radial force against the inside wall of a vein to anchor the device within the vein. Further, the implant device can tend to minimize backflow of blood or venous insufficiency. Examples of treatment applications for the device are iliofemoral venous obstruction, and chronic iliac venous outflow obstruction as a result of venous disease. The device can also be used to provide temporary or permanent occlusion of a vessel during and/or after treatment of a tumor by intravascular injection of fluids, chemotherapy drugs, liquid embolic agents, and/or other therapeutic agents delivered into the feeding vessels and/or into the tumor.

### Additional Features

Features of any of the implants, the support frames, and/or the membranes disclosed herein can be applied to other devices and implants disclosed herein. Any implant of the present disclosure may be configured to interact with structures of an engagement structure of a catheter or delivery device disclosed herein. Features of a membrane disclosed herein can be applied to other membranes or implants disclosed herein.

Some embodiments are also provided by which the assembly and/or catheter can be advanced over a guidewire, thus allowing treatment of more tortuous or distal, smaller vessels in the vasculature. Other features and characteristics of the assembly and/or catheter can be modified to include any of the structures or features discussed above, or as those disclosed in: U.S. patent application Ser. No. 12/826,593, filed on Jun. 29, 2010 (086538-0012); U.S. patent application Ser. No. 13/367,338, filed on Feb. 6, 2012 (086538-0018); U.S. patent application Ser. No. 12/906,993, filed on Oct. 18, 2010 (086538-0014); U.S. patent application Ser. No. 13/828,974, filed on Mar. 14, 2013 (086538-0030); U.S. Patent App. No. 61/836,061, filed on Jun. 17, 2013 (086538-0038); U.S. patent application Ser. No. 14/044,794, filed on Oct. 2, 2013 (086538-0039); U.S. patent application Ser. No. 14/281,797, filed on May 19, 2014 (086538-0055); U.S. Patent App. No. 61/835,406, filed on Jun. 14, 2013 (086538-0032); U.S. Patent App. No. 61/904,376, filed on Nov. 14, 2013 (086538-0041); U.S. Patent App. No. 61/904,379, filed on Nov. 14, 2013 (086538-0043); U.S. Patent App. No. 61/835,461, filed on Jun. 14, 2013 (086538-0034); U.S. Patent App. No. 61/900,321, filed on Nov. 5, 2013 (086538-0040); U.S. patent application Ser. No. 14/101,171, filed on Dec. 9, 2013 (086538-0046); U.S. Patent App. No. 61/987,446, filed on May 1, 2014 (086538-0054); and U.S. patent application Ser. No. 14/304,868, filed on Jun. 13, 2014 (086538-0057), the entireties of which are incorporated herein by reference.

According to some embodiments of the subject technology, the support frame may comprise at least one of stainless steel, nickel titanium (NiTi), cobalt chromium (CoCr), titanium, a polymer, a polyester based material, a tyrosine based polycarbonate, a polyethylene based material, Teflon (e.g., including expanded Teflon), and other suitable materials known to those of ordinary skill in the art. In some embodiments, support frame may comprise at least one of polyethylene, polyglicolide, polylactide, ε-caprolactone, polycarbonate, hydroxyalkanote, para dioxinine, polytetrafluoroethylene (PTFE), expanded polytetrafluoroethylene (ePTFE), PLA, PGA, PLLA, PDLLA, PDO, PCL, and other suitable materials known to those of ordinary skill in the art. In some embodiments, support frame and/or occlusion membrane, may comprise a bioabsorbable material, beneficially allowing for their controlled degradation. In some embodiments, support frame and/or occlusion membrane may be formed of bioabsorbable material to have a controlled degradation anywhere between about 3 months to about 3 years depending on the desired application of support frame. In some embodiments, the controlled degradation may be less than about 3 months or greater than about 3 years. For example, hydrolysis of ester linkages or effects of enzymatic degradation may be utilized for the controlled degradation.

In some embodiments, the support frame may be coated with various suitable agents to allow support frame to expand within and engage the inner surface of the vessel or lumen. For example, support frame may be coated with biological glue. In some embodiments, support frame may be coated with a friction-resistant coating (e.g., a friction-resistant polymer coating). In some embodiments, radio-opaque markers may be located on support frame or occlusion membrane for endovascular or other image-guided procedures. In some embodiments, the radio-opaque marker may be a platinum iridium alloy or other suitable markers known to those of ordinary skill in the art.

According to various embodiments of the subject technology, occlusion membrane may be used to occlude, partially or completely, luminal structure in which an implant is deployed. In some embodiments as used herein, occlusion may refer to either partial or complete occlusion.

According to some embodiments, implants disclosed herein can incorporate any one or more of the features disclosed in the figures or discussion herein. For example, any of the implants can be configured to comprise a fibrous membrane feature, as disclosed in Applicant's copending U.S. patent application Ser. No. 14/304,868, filed on Jun. 13, 2014 (086538-0057), the entirety of the disclosure of which is incorporated herein by reference.

According to some embodiments, implants disclosed herein can have an expanded diameter of between about 4 mm to about 22 mm. Additionally, some embodiments can be used in vessels having diameters between about 3 mm to about 20 mm.

According to some embodiments, implants disclosed herein can be deployed in vessels having dimensions of between about 3 mm to about 20 mm. The target delivery profile can be about 8 Fr, about 7 Fr, about 6 Fr, about 5 Fr, about 4 Fr, about 3 Fr, or smaller.

Furthermore, implants disclosed herein can also be configured for use in venous stenting and can comprise any of the features taught herein to facilitate such use, including incorporating a fibrous membrane into the implant frame. For example, stenting of vessels having diameters between about 3 mm to about 20 mm can be possible using embodiments disclosed herein. This exceptional and advantageous ability of embodiments of the medical implants disclosed herein to provide stenting in such small vessels is made possible, for example, due to the minimal delivery profile can be achieved using such embodiments. As noted above with other embodiments, deployment of an implant having a fibrous membrane feature can exert an outward radial force against inside wall of a vein in order to improve blood flow, or minimize vein insufficiency. Further, the delivery profile can be about 8 Fr or smaller, as discussed herein.

According to various aspects of the subject technology, implants disclosed herein may be used for various applications for reducing or stopping flow through a luminal structure in a patient. Implants of the subject technology may be used for rapid, well-controlled, and reliable occlusion of luminal structures. For example, the luminal structure may comprise at least one of a blood vessel, a body organ, a lung, an airway, a Fallopian tube, a cervical canal, a vagina, a cervix, a vas deferens, a bronchus, a ureter, a colon, a rectum, an anus, a bio duct, a pancreatic duct, or other suitable tubular structures known to those of ordinary skill in the art. In some embodiments, implants of the present disclosure may be used for temporary occlusion in cases of lung disease, or for temporary occlusion of female reproductive organs for contraceptive purposes. In some embodiments, implants of the present disclosure may be removed, or flow may be restored through the luminal structure to restore original organ functions.

In some embodiments, implants of the present disclosure may be used for various endoluminal occlusion procedures, including procedures for the lungs (e.g., selective endobronchial occlusion for lung reduction, occlusion of bronchopleural or bronchocutaneous fistulas, endovascular occlusion of pulmonary AVMs and fistulas or aortopulmonary anastomoses) and procedures for reproductive organs (e.g., endoluminal occlusion of vas deferens or Fallopian tubes for minimally-invasive contraceptive intervention, endovascular occlusion of varicocele in males and low abdominal gonadal veins for reducing or completely eliminating chronic pelvic pain syndrome in females). In some embodiments, implants of the present disclosure may be used for stopping blood loss from a damaged blood vessel, closing an abnormal blood vessel or a blood vessel supplying a vascular anomaly, or interrupting blood supply to an organ or part of an organ for permanent devascularization (e.g., closure of splenic artery in spleen laceration, devascularization of tissues involved by neoplastic process, either pre-operatively or as a palliative measure). In some embodiments, implants of the present disclosure may be used for various endovascular (e.g., neural and peripheral) procedures including procedures for giant cerebral and skull base aneurysms (ruptured and non-ruptured), head and neck arteriovenous fistulas, dissecting intracranial and extracranial vessels, traumatic and non-traumatic vessel injury or rupture (e.g., pelvic hemorrhages in trauma patients, carotid blow-out in patients with head and neck cancers, hemorrhage induced by a neoplasia, etc.), and devascularization prior to (or as an alternative to) surgical resection of various organs or tumors.

In certain embodiments, implants of the present disclosure may be used for various organs, including for example, the spleen (e.g., endovascular occlusion as a preoperative intervention or as an alternative to surgical resection with indications including traumatic hemorrhage, hypersplenism, bleeding secondary to portal hypertension or splenic vein thrombosis, and various disorders such as thalassemia major, thrombocytopenia, idiopathic thrombocytopenic purpura, Gaucher disease, and Hodgkin disease), the liver (e.g., occlusion of portal veins collaterals as adjunct to a transjugular intrahepatic portosystemic shunt (TIPS), occlusion of the TIPS itself in cases of encephalopathy, occlusion of intrahepatic arterioportal fistulas), the kidney (e.g., endoluminal ureteral occlusion for intractable lower urinary tract fistula with urine leakage, or for the treatment of uretero-arterial fistulae, endovascular occlusion as an alternative to surgical resection for end-stage renal disease or renovascular hypertension requiring unilateral or bilateral nephrectomy and renal transplant with native kidneys in situ), and the heart (e.g., occlusion of coronary arteriovenous fistulas, transarterial embolization of Blalock-Taussig shunts). The application of implants of the present disclosure is not limited to applications for human patients, but may also include veterinary applications.

According to some embodiments, covers (including patches) disclosed herein and in the above-noted patent applications, including but not limited to the cover member 1132, can be attached to a respective implant. Covers disclosed herein may be attached to one or both ends or an implant and/or a middle region of an implant.

According to various embodiments of the subject technology, a cover component of an implant may be used to occlude, partially or completely, luminal structure in which a respective implant is deployed. In some embodiments as used herein, occlusion may refer to either partial or complete occlusion. In some embodiments, cover components can comprise at least one of a polyurethane, a polyanhidrate, PTFE, ePTFE, silicone, and other suitable materials known to those of ordinary skill in the art. In some embodiments, cover components may be elastic. In some embodiments, cover components may be permeable or non-permeable.

In some embodiments, an average thickness of a cover component can be between about 0.0005 inches and about 0.006 inches. In some aspects, the average thickness of a cover component may be less than about 0.0005 inches or greater than about 0.006 inches. In certain embodiments, an average thickness of a distal portion of a cover component is greater than an average thickness of a proximal portion of a cover component. Such a configuration may ensure that more flow may be reduced at the distal portion of a cover component. In some embodiments, the average thickness of the distal portion of a cover component is between about 0.002 inches and about 0.012 inches. In some embodiments, the average thickness of the distal portion of a cover component may be less than about 0.002 inches or greater than about 0.012 inches. In some embodiments, the average thickness of the proximal portion of a cover component is between about 0.0005 inches and about 0.006 inches. In some embodiments, the average thickness of the proximal portion of a cover component may be less than about 0.0005 inches or greater than about 0.006 inches.

### Additional Disclosures

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method Claims and clauses present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the Claims, such term is intended to be inclusive in a manner similar to the term “comprise” as “comprise” is interpreted when employed as a transitional word in a Claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the inventions have been described, these have been presented by way of example only, and are not intended to limit the scope of the inventions. Indeed, the novel methods and systems described herein may be embodied in a variety of other forms without departing from the spirit thereof. The accompanying Claims and their equivalents are intended to cover such forms or modifications as would fall within the scope and spirit of the inventions.

What is claimed is:

1. A medical device for implantation into a body lumen, the device comprising a frame and a cover component, the frame comprising first and second expandable loops that are interconnected by a backbone portion extending between the first and second loops, wherein interconnections between the backbone portion and the first and second loops bias the first and second loops from a collapsed, substantially linear configuration to an expanded configuration in which the first and second loops extend transversely relative to the backbone portion and substantially parallel relative to each other, the first and second loops having expanded diameters in the expanded configuration, the cover component comprising a substantially tubular member having a collapsed diameter when surrounding the frame in the collapsed configuration, the cover component comprising expandable portions longitudinally aligned with the first and second loops in the collapsed configuration and a substantially non-expandable portion longitudinally interposed along between the first and second loops along the backbone portion, wherein in the expanded configuration, the expandable portions expand from the collapsed diameter to the expanded diameters of the first and second loops and the substantially non-expandable portion remains in the collapsed diameter.

2. The device of claim 1, wherein the frame comprises a proximal coupling member for engaging with a portion of a delivery device.

3. The device of claim 1, wherein the frame comprises a pair of wires diverging away from each other and converging toward each other to form the first and second loops.

4. The device of claim 3, wherein the pair of wires are welded together along the backbone portion of the frame.

5. The device of claim 3, wherein the pair of wires (i) converge at the interconnections between the backbone portion and the first and second loops and (ii) diverge between the interconnections along a longitudinal extent of the backbone portion to form the first and second loops.

6. The device of claim 3, wherein each of the wires extends in a semi-circular arc along the backbone portion.

7. The device of claim 1, wherein expanded diameters of the expandable portions are between about 6 times and about 30 times the collapsed diameter.

8. The device of claim 1, wherein expandable portions comprise pre-stretched sections of the cover component.

9. The device of claim 1, wherein expandable portions comprise additional folds of material to facilitate expansion to the expanded diameters.

10. The device of claim 1, wherein expandable portions comprise collapsed diameters greater than the collapsed diameter of the non-expanding portion.

11. A medical device delivery system, comprising:
a catheter comprising proximal and distal portions and first and second lumens, the first lumen extending from the proximal portion to a first aperture at the distal portion, the second lumen extending from the proximal portion to a second aperture at the distal portion, the first aperture distal to the second aperture;
a first elongate actuation member, longitudinally moveable within the first lumen, comprising a first distal coupling section;
a second elongate actuation member, longitudinally moveable within the second lumen, comprising a second distal coupling section; and
a medical device extending along an outer surface of the catheter distal portion in a collapsed configuration, the device having (i) a proximal engagement portion extending proximally through the second aperture into the second lumen and releasably coupled to the second actuation member, and (ii) a distal engagement portion extending proximally through the first aperture into the first lumen to releasably couple the distal engagement portion to the distal portion;
wherein distal advancement of the first and second actuation members releases the proximal and distal engagement portions from the first and second lumens, thereby permitting expansion of the device from the collapsed configuration.

12. The system of claim 11, wherein the first actuation member is distally advanceable within the first lumen to push the distal engagement portion out of the first lumen, thereby releasing the distal engagement portion to permit expansion of the device from the collapsed configuration.

13. The system of claim 11, wherein the distal engagement portion comprises an elongate body having a longitudinal axis that is substantially parallel or coaxial relative to the first lumen when the elongate body is positioned within the first lumen.

14. The system of claim 11, wherein the proximal engagement portion comprises an elongate body having a longitudinal axis that is substantially parallel or coaxial relative to the second lumen when the elongate body is positioned within the second lumen.

15. The system of claim 11, wherein the distal engagement portion radially overlaps with the first distal coupling section when the distal engagement portion extends within the first lumen.

16. The system of claim 11, wherein the distal engagement portion comprises a notch or a protrusion that radially engages with a notch or a protrusion of the first distal coupling section when the distal engagement portion extends within the first lumen to constrain axial motion of the distal engagement portion relative to the first distal coupling section.

17. The system of claim 16, wherein the proximal engagement portion comprises a notch or a protrusion that radially engages with a notch or a protrusion of the second distal coupling section when the proximal engagement portion extends within the second lumen to constrain axial motion of the proximal engagement portion relative to the second distal coupling section.

18. The system of claim 11, wherein the distal engagement portion comprises a U-shaped body having a first portion coupled to the device and a free, second portion configured to extend into the first lumen.

19. A medical device frame for implantation into a body lumen, the frame comprising proximal and distal engagement portions and an expandable central portion disposed between the proximal and distal engagement portions, the proximal engagement portion comprising an elongate proximal coupling member having a radial notch, the distal engagement portion comprising an elongate distal coupling member having a radial notch, the distal engagement portion having a bend such that the distal coupling member extends from the distal engagement portion in a proximal direction, wherein the frame comprises a pair of wires forming first and second backbone portions and a hoop section disposed intermediate the first and second backbone portions, the pair of wires being (i) welded together in the first backbone portion, (ii) diverging away from each other to form the hoop section, and (iii) converging toward each other to be welded together in the second backbone portion.

20. The device frame of claim 19, wherein the frame comprises a distal hoop section.

21. The device frame of claim 20, wherein in an open configuration, a longitudinal axis of the device frame extends at an angle of between about 30 degrees and about 90 degrees relative to a hoop plane through which the distal hoop section passes.

22. The device frame of claim 21, wherein in a closed configuration, the longitudinal axis extends at an angle of between about 0 degrees and about 10 degrees relative to the hoop plane.

23. The device frame of claim 19, wherein the frame comprises a plurality of hoop sections.

24. The device frame of claim 19, wherein the central portion comprises a plurality of hoop sections interconnected by longitudinal backbones.

25. The device frame of claim 19, further comprising a tubular cover member having an open proximal end portion and a distal end portion, the distal end portion comprising an aperture extending therethrough, the cover member coupled to the frame.

26. The device frame of claim 25, wherein the frame is positioned inside the cover member.
